SEQUENCE LISTING (66 sequences)

SEQ ID NO: 1 - protein, 13 aa, Human immunodeficiency virus 1
GRKKRRQRRRPPQ

SEQ ID NO: 2 - protein, 17 aa, Human immunodeficiency virus 1
GISYGRKKRRQRRRPPQ

SEQ ID NO: 3 - protein, 23 aa, Human immunodeficiency virus 1
FITKALGISYGRKKRRQRRRPPQ

SEQ ID NO: 4 - protein, 10 aa, Human immunodeficiency virus 1
GRKKRRQRRR

SEQ ID NO: 5 - protein, 9 aa, Human immunodeficiency virus 1
RKKRRQRRR

SEQ ID NO: 6 - protein, 8 aa, Human immunodeficiency virus 1
RKKRRQRR

SEQ ID NO: 7 - protein, 9 aa, Human immunodeficiency virus 1
RRRQRRKKR

SEQ ID NO: 8 - protein, 9 aa, Human immunodeficiency virus 1
RKKRRARRR

SEQ ID NO: 9 - protein, 11 aa, Human immunodeficiency virus 1
GRKKRRQRRRC

SEQ ID NO: 10 - protein, 17 aa, Human immunodeficiency virus 1
TRQARRNRRRRWRERQR

SEQ ID NO: 11 - protein, 30 aa, Unknown
  Description of Unknown Galanin CPP peptide
GWTLNSAGYLLGPHAVGNHRSFSDKNGLTS

SEQ ID NO: 12 - protein, 14 aa, Vespula lewisii
INLKALAALAKKIL

SEQ ID NO: 13 - protein, 16 aa, Drosophila sp.
RQIKIWFQNRRMKWKK

SEQ ID NO: 14 - protein, 16 aa, Drosophila sp.
RQIKIWFQNRRMKWKK

SEQ ID NO: 15 - protein, 16 aa, Drosophila sp.
KKWKMRRNQFWIKIQR

SEQ ID NO: 16 - protein, 16 aa, Drosophila sp.
RQIKIWFPNRRMKWKK

SEQ ID NO: 17 - protein, 16 aa, Drosophila sp.
RQPKIWFPNRRKPWKK

SEQ ID NO: 18 - protein, 16 aa, Drosophila sp.
RQIKIWFQNRRMKWKK

SEQ ID NO: 19 - protein, 15 aa, Drosophila sp.
RQIKIWFQNRRMKWK

SEQ ID NO: 20 - protein, 14 aa, Drosophila sp.
RQIKIWFQNRRMKW

SEQ ID NO: 21 - protein, 14 aa, Drosophila sp.
IKIWFQNRRMKWKK

SEQ ID NO: 22 - protein, 16 aa, Drosophila sp.
RQIKIWFPNRRMKWKK

SEQ ID NO: 23 - protein, 10 aa, Unknown
  Description of Unknown PTD4 CPP peptide
RAAARQARAG

SEQ ID NO: 24 - protein, 12 aa, Unknown
  Description of Unknown PTD4 CPP peptide
YARAAARQARAG

SEQ ID NO: 25 - protein, 13 aa, Crotalus durissus
KMDCRWRWKCCKK

SEQ ID NO: 26 - protein, 16 aa, Homo sapiens
RKKRRRESRKKRRRES

SEQ ID NO: 27 - protein, 17 aa, Homo sapiens
GRPRESGKKRKRKRLKP

SEQ ID NO: 28 - protein, 15 aa, Homo sapiens
GKRKKKGKLGKKRDP

SEQ ID NO: 29 - protein, 19 aa, Unknown
  Description of Unknown DPV10/6 CPP peptide
SRRARRSPRESGKKRKRKR

SEQ ID NO: 30 - protein, 5 aa, Homo sapiens
VPMLK

SEQ ID NO: 31 - protein, 5 aa, Homo sapiens
KLPVM

SEQ ID NO: 32 - protein, 22 aa, Mycobacterium tuberculosis
TKRRITPKDVIDVRSVTTEINT

SEQ ID NO: 33 - protein, 26 aa, Mycobacterium tuberculosis
AEKVDPVKLNLTLSAAAEALTGLGDK

SEQ ID NO: 34 - protein, 22 aa, Mycobacterium tuberculosis
TKRRITPKDVIDVRSVTTKINT

SEQ ID NO: 35 - protein, 29 aa, Unknown
  Description of Unknown Res1 L3 loop of restrictocin CPP peptide
KLIKGRTPIKFGKADCDRPPKHSQNGMGK

SEQ ID NO: 36 - protein, 37 aa, Human respiratory syncytial virus
KRIPNKKPGKKTTTKPTKKPTIKTTKKDLKPQTTKPK

SEQ ID NO: 37 - protein, 22 aa, Unknown
  Description of Unknown RSG 1.2 Arg-rich CPP peptide
DRRRRGSRPSGAERRRRRAAAA

SEQ ID NO: 38 - protein, 25 aa, Homo sapiens
GTKMIFVGIKKKEERADLIAYLKKA

SEQ ID NO: 39 - protein, 17 aa, Flock house virus
RRRRNRTRRNRRRVRGC

SEQ ID NO: 40 - protein, 11 aa, Homo sapiens
MIIYRDLISKK

SEQ ID NO: 41 - protein, 10 aa, Homo sapiens
MIIYRDKKSH

SEQ ID NO: 42 - protein, 10 aa, Homo sapiens
MIIFRDLISH

SEQ ID NO: 43 - protein, 10 aa, Homo sapiens
MIIYRDLISH

SEQ ID NO: 44 - protein, 8 aa, Artificial Sequence
  Description of Artificial Sequence Synthetic peptide
RRRRRRRR

SEQ ID NO: 45 - protein, 9 aa, Artificial Sequence
  Description of Artificial Sequence Synthetic peptide
RRRRRRRRR

SEQ ID NO: 46 - protein, 27 aa, Unknown
  Description of Unknown Transportan (TP) CPP peptide
GWTLNSAGYLLGKINLKALAALAKKIL

SEQ ID NO: 47 - protein, 20 aa, Artificial Sequence
  Description of Artificial Sequence Synthetic peptide
ALWKTLLKKVLKAPKKKRKV

SEQ ID NO: 48 - protein, 13 aa, Artificial Sequence
  Description of Artificial Sequence Synthetic peptide
EEEAAGRKRKKRT

SEQ ID NO: 49 - protein, 21 aa, Unknown
  Description of Unknown Pep-1 CPP peptide
KETWWETWWTEWSQPKKKRKV

SEQ ID NO: 50 - protein, 18 aa, Homo sapiens
GLRRLRQRRRLRRERVRA

SEQ ID NO: 51 - protein, 16 aa, Unknown
  Description of Unknown CPP peptide
AAVALLPAVLLALLAP

SEQ ID NO: 52 - protein, 15 aa, Unknown
  Description of Unknown CPP peptide
KWKLFKKIGAVLKVL

SEQ ID NO: 53 - protein, 11 aa, Unknown
  Description of Unknown CPP peptide
KKLFKKILKYL

SEQ ID NO: 54 - protein, 16 aa, Unknown
  Description of Unknown CPP peptide
DGPKKKKKKSPSKSSG

SEQ ID NO: 55 - protein, 16 aa, Unknown
  Description of Unknown CPP peptide
GSSKSPSKKKKKKPGD

SEQ ID NO: 56 - protein, 20 aa, Unknown
  Description of Unknown CPP peptide
SPSNETPKKKKKRFSFKKSG

SEQ ID NO: 57 - protein, 16 aa, Unknown
  Description of Unknown CPP peptide
DGPKKKKKKSPSKSSK

SEQ ID NO: 58 - protein, 14 aa, Unknown
  Description of Unknown CPP peptide
SKDGKKKKKKSKTK

SEQ ID NO: 59 - protein, 6 aa, Artificial Sequence
  Description of Artificial Sequence Synthetic 6xHis tag
HHHHHH

SEQ ID NO: 60 - protein, 4 aa, Artificial Sequence
  Description of Artificial Sequence Synthetic peptide
GGGS

SEQ ID NO: 61 - protein, 5 aa, Artificial Sequence
  Description of Artificial Sequence Synthetic peptide
GGGGS

SEQ ID NO: 62 - protein, 1676 aa, Homo sapiens
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 63 - protein, 74 aa, Homo sapiens
TLQKKIEEIAAKYKHSVVKKCCYDGACVNNDETCEQRAARISLGPRCIKAFTECCVVASQLRANISHKDMQLGR

SEQ ID NO: 64 - protein, 350 aa, Homo sapiens
MNSFNYTTPDYGHYDDKDTLDLNTPVDKTSNTLRVPDILALVIFAVVFLVGVLGNALVVWVTAFEAKRTINAIWFLNLAVADFLSCLALPILFTSIVQHHHWPFGGAACSILPSLILLNMYASILLLATISADRFLLVFKPIWCQNFRGAGLAWIACAVAWGLALLLTIPSFLYRVVREEYFPPKVLCGVDYSHDKRRERAVAIVRLVLGFLWPLLTLTICYTFILLRTWSRRATRSTKTLKVVVAVVASFFIFWLPYQVTGIMMSFLEPSSPTFLLLNKLDSLCVSFAYINCCINPIIYVVAGQGFQGRLRKSLPSLLRNVLTEESVVRESKSFTRSTVDTMAQKTQAV

SEQ ID NO: 65 - protein, 337 aa, Homo sapiens [sequence omitted]

SEQ ID NO: 66 - protein, 443 aa, Homo sapiens
MDFPCLWLGLLLPLVAALDFNYHRQEGMEAFLKTVAQNYSSVTHLHSIGKSVKGRNLWVLVVGRFPKEHRIGIPEFKYVANMHGDETVGRELLLHLIDYLVTSDGKDPEITNLINSTRIHIMPSMNPDGFEAVKKPDCYYSIGRENYNQYDLNRNFPDAFEYNNVSRQPETVAVMKWLKTETFVLSANLHGGALVASYPFDNGVQATGALYSRSLTPDDDVFQYLAHTYASRNPNMKKGDECKNKMNFPNGVTNGYSWYPLQGGMQDYNYIWAQCFEITLELSCCKYPREEKLPSFWNNNKASLIEYIKQVHLGVKGQVFDQNGNPLPNVIVEVQDRKHICPYRTNKYGEYYLLLLPGSYIINVTVPGHDPHITKVIIPEKSQNFSALKKDILLPFQGQLDSIPVSNPSCPMIPLYRNLPDHSAATKPSLFLFLVSLLHIFFK

(19) United States
(12) Patent Application Publication
(10) Pub. No.: US 2019/0201488 A1
(43) Pub. Date: Jul. 4, 2019

(54) COMPOSITIONS AND METHODS RELATING TO C5L2

(71) Applicants: King's College London, London (GB); Apellis Pharmaceuticals, Inc., Crestwood, KY (US)

(72) Inventors: Claudia Kemper, Greenwich (GB); Cedric Francois, Prospect, KY (US); Pascal Deschatelets, Prospect, KY (US); Monica Gerber, Somerville, MA (US)

(21) Appl. No.: 15/887,939

(22) Filed: Feb. 2, 2018

Related U.S. Application Data
(63) Continuation of application No. 14/910,833, filed on Feb. 8, 2016, now abandoned.
(60) Provisional application No. 61/868,016, filed on Aug. 20, 2013.
(60) Provisional application No. 61/864,510, filed on Aug. 9, 2013.

(51) Int. Cl. A61K 38/17; A61K 47/64; C07K 14/57; C07K 14/47; G01N 33/50; G01N 33/564; G01N 33/68; C12N 15/113; C07K 14/54; A61K 45/00; A61K 38/48; C12N 15/115; A61K 45/06; C12N 5/0783

(52) U.S. Cl. CPC A61K 38/1725; A61K 39/00; C07K 14/57; C07K 14/472; G01N 33/5023; G01N 33/505; G01N 33/564; G01N 33/6866; G01N 33/6869; C12N 15/113; C07K 14/54; A61K 45/00; A61K 38/4813; C12N 15/115; A61K 45/06; C12N 5/0636; G01N 2333/54; G01N 2333/57; C12N 2310/16; C12N 2310/14; C12N 2320/30; C12Y 304/17012; G01N 2333/4716; A61K 47/645

(57) ABSTRACT

In some aspects, provided herein is a method of enhancing production of interleukin-17 (IL-17), interferon gamma (IFN-γ), or both by a mammalian T cell, the method comprising contacting the cell with a C5L2 inhibitor. In some aspects, provided herein is a method of enhancing Th1 and/or Th17 responses by a mammalian T cell, the method comprising contacting the cell with a C5L2 inhibitor. In some aspects, provided herein is a method of enhancing production of interleukin-6 (IL-6), interleukin 1 beta (IL-1β), or both by a mammalian T cell or monocyte, the method comprising contacting the cell with a C5L2 inhibitor. In some aspects, provided herein is a method of decreasing suppressive activity of a T regulatory cell, e.g., a natural regulatory T (nTreg) cell, the method comprising contacting a Treg cell, e.g., an nTreg cell, with an inhibitor of C5L2.

### CROSS REFERENCE TO RELATED APPLICATIONS

This is a continuation patent application of U.S. patent application Ser. No. 14/910,833, filed Feb. 8, 2016, which is a National Phase application under 35 U.S.C. § 371 of International Application No. PCT/US2014/050389, filed Aug. 8, 2014, which claims the benefit of and priority to each of U.S. Provisional Patent Application Ser. No. 61/868,016, filed Aug. 20, 2013 and U.S. Provisional Patent Application Ser. No. 61/864,510, filed Aug. 9, 2013, the entire contents of each of which are hereby incorporated by reference.

### SEQUENCE LISTING

The present specification makes reference to a Sequence Listing (submitted electronically as a .txt file named “2008575-0079_SL.txt on May 24, 2016). The .txt file was generated on Sep. 17, 2014 and is 39,247 bytes in size. The entire contents of the Sequence Listing are herein incorporated by reference.

### BACKGROUND

Complement is an arm of the immune system that plays an important role in defending the body against infectious agents. The complement system comprises more than 30 serum and cellular proteins that are involved in three major pathways, known as the classical, alternative, and lectin pathways. The classical pathway is usually triggered by binding of a complex of antigen and IgM or IgG antibody to C1 (though certain other activators can also initiate the pathway). Activated C1 cleaves C4 and C2 to produce C4a and C4b, in addition to C2a and C2b. C4b and C2a combine to form C3 convertase, which cleaves C3 to form C3a and C3b. Binding of C3b to C3 convertase produces C5 convertase, which cleaves C5 into C5a and C5b. C3a, C4a, and C5a are anaphylotoxins and mediate multiple reactions in the acute inflammatory response. C3a and C5a are also chemotactic factors that attract immune system cells such as neutrophils.

The alternative pathway is initiated by and amplified at, e.g., microbial surfaces and various complex polysaccharides. In this pathway, hydrolysis of C3 to C3(H2O), which occurs spontaneously at a low level, leads to binding of factor B, which is cleaved by factor D, generating a fluid phase C3 convertase that activates complement by cleaving C3 into C3a and C3b. C3b binds to targets such as cell surfaces and forms a complex with factor B, which is then cleaved by factor D, resulting in a C3 convertase. Surface-bound C3 convertases cleave and activate additional C3 molecules, resulting in rapid C3b deposition in close proximity to the site of activation and leading to formation of additional C3 convertase, which in turn generates additional C3b. This process results in a cycle of C3 cleavage and C3 convertase formation that significantly amplifies the response. Cleavage of C3 and binding of another molecule of C3b to the C3 convertase gives rise to a C5 convertase. C3 and C5 convertases of this pathway are regulated by host cell molecules CR1, DAF, MCP, CD59, and fH. The mode of action of these proteins involves either decay accelerating activity (i.e., ability to dissociate convertases), ability to serve as cofactors in the degradation of C3b or C4b by factor I, or both. Normally the presence of complement regulatory proteins on host cell surfaces prevents significant complement activation from occurring thereon.

The C5 convertases produced in both pathways cleave C5 to produce C5a and C5b. C5b then binds to C6, C7, and C8 to form C5b-8, which catalyzes polymerization of C9 to form the C5b-9 membrane attack complex (MAC). The MAC inserts itself into target cell membranes and causes cell lysis. Small amounts of MAC on the membrane of cells may have a variety of consequences other than cell death.

The lectin complement pathway is initiated by binding of mannose-binding lectin (MBL) and MBL-associated serine protease (MASP) to carbohydrates. The MB1-1 gene (known as LMAN-1 in humans) encodes a type I integral membrane protein localized in the intermediate region between the endoplasmic reticulum and the Golgi. The MBL-2 gene encodes the soluble mannose-binding protein found in serum. In the human lectin pathway, MASP-1 and MASP-2 are involved in the proteolysis of C4 and C2, leading to a C3 convertase described above.

Complement activity is normally regulated by mammalian proteins referred to as complement control proteins (CCPs) or regulators of complement activation (RCA) proteins. These proteins normally serve to limit complement activation that might otherwise occur on cells and tissues of the mammalian, e.g., human host. CCPs are characterized by the presence of multiple (typically 4-56) homologous motifs known as short consensus repeats (SCR), complement control protein (CCP) modules, or SUSHI domains, about 50-70 amino acids in length that contain a conserved motif including four disulfide-bonded cysteines (two disulfide bonds), proline, tryptophan, and many hydrophobic residues. CCPs include complement receptor type 1 (CR1), complement receptor type 2 (CR2), membrane cofactor protein (MCP; CD46), decay-accelerating factor (DAF), complement factor H (fH), and C4b-binding protein (C4bp). CD59 is a membrane-bound complement regulatory protein unrelated structurally to the CCPs.

Recently, a number of important interactions between the complement system and components of the adaptive immune system have been uncovered.

### SUMMARY

In some aspects, provided herein is a method of enhancing production of interleukin-17 (IL-17), interferon gamma (IFN-γ), or both by a mammalian T cell, the method comprising contacting the cell with a C5L2 inhibitor. In some embodiments the T cell is a CD4+ T cell. In some embodiments a CD4+ cell is a resting CD4+ T cell. In some embodiments a CD4+ cell is an activated CD4+ T cell.

In some aspects, provided herein is a method of enhancing Th1 and/or Th17 responses by a mammalian T cell, the method comprising contacting the cell with a C5L2 inhibitor. In some embodiments the T cell is a CD4+ T cell. In some embodiments a CD4+ cell is a resting CD4+ T cell. In some embodiments a CD4+ cell is an activated CD4+ T cell.

In some aspects, provided herein is a method of enhancing production of interleukin-6 (IL-6), interleukin 1 beta (IL-1β), or both by a mammalian T cell or monocyte, the method comprising contacting the cell with a C5L2 inhibitor. In some embodiments the T cell is a CD4+ T cell. In some embodiments the cell is a resting CD4+ T cell or resting monocyte. In some embodiments the cell is an activated CD4+ T cell or activated monocyte.

In some aspects, provided herein is a method of decreasing suppressive activity of a T regulatory cell, e.g., a natural regulatory T (nTreg) cell, the method comprising contacting a Treg cell, e.g., an nTreg cell, with an inhibitor of C5L2.

In some embodiments contacting a cell with a C5L2 inhibitor comprises contacting the cell with the C5L2 inhibitor in vivo. In some embodiments contacting a cell with a C5L2 inhibitor in vivo comprises administering the C5L2 inhibitor to a mammalian subject. In some embodiments contacting a cell with a C5L2 inhibitor in vivo comprises administering the C5L2 inhibitor to a mammalian subject who may benefit from increased production of IL-17 and/or IFN-γ. In some embodiments a subject who may benefit from increased production of IL-17 and IFN-γ is in need of treatment for an infection or cancer. In some embodiments contacting a cell with a C5L2 inhibitor in vivo comprises administering the C5L2 inhibitor to a mammalian subject who may benefit from increased Th1 and/or Th17 responses. In some embodiments a subject who may benefit from increased Th1 and/or Th17 responses is in need of treatment for an infection or cancer. In some embodiments contacting a cell with a C5L2 inhibitor in vivo comprises administering the C5L2 inhibitor to a mammalian subject who may benefit from increased production of IL-6 and/or IL-1β. In some embodiments a subject who may benefit from increased production of IL-6 and/or IL-1β is in need of treatment for an infection or cancer. In some embodiments contacting a cell with a C5L2 inhibitor in vivo comprises administering the C5L2 inhibitor to a mammalian subject who may benefit from a decrease in suppressive activity of nTreg cells. In some embodiments a subject who may benefit from a decrease in suppressive activity of nTreg cells is in need of treatment for an infection or cancer.

In some embodiments of any aspect relating to a C5L2 inhibitor, the C5L2 inhibitor comprises an antibody, an engineered non-antibody polypeptide, a peptide, a peptidomimetic, or a small molecule. In some embodiments of any aspect relating to a C5L2 inhibitor, the C5L2 inhibitor comprises a variant of C5a, optionally comprising a substitution at position 69 of C5a. In some embodiments of any aspect relating to a C5L2 inhibitor the C5L2 inhibitor comprises a variant of C5a comprising a positively charged amino acid at position 69 (such as arginine), optionally having a deletion of amino acid 74 of C5a, further optionally having deletion or substitution at one or more of positions 71-73 of C5a, further optionally wherein the variant is A8 or A8Delta71-73. In some embodiments of any aspect relating to a C5L2 inhibitor, the C5L2 inhibitor is a C5L2 antagonist, optionally a C5aR/C5L2 receptor dual antagonist. In some embodiments a C5L2 antagonist is selective for C5L2 as compared with C5aR. In some embodiments of any aspect relating to a C5L2 inhibitor, the C5L2 inhibitor is an agent that inhibits a T cell expressed enzyme that processes C5a into C5adesArg. In some embodiments of any aspect relating to a C5L2 inhibitor, the C5L2 inhibitor comprises an agent that inhibits carboxypeptidase M. In some embodiments of any aspect relating to a C5L2 inhibitor, the C5L2 inhibitor comprises a nucleic acid, wherein the nucleic acid optionally comprises a RNAi agent (e.g., an siRNA) that inhibits expression of C5L2 or carboxypeptidase M (CPM) or comprises an aptamer that binds to C5L2 or CPM.

In some aspects, provided herein is a method of identifying a candidate inhibitor of C5L2, comprising contacting a mammalian T cell with a test agent and determining whether the test agent increases production of IL-17, IFN-γ, or both, by the T cell, wherein an agent that increases production of IL-17, IFN-γ, or both, by the T cell, is a candidate inhibitor of C5L2. In some embodiments the T cell is a CD4+ T cell.

In some aspects, provided herein is a method of identifying an enhancer of Th1 and/or Th17 responses, comprising identifying a C5L2 inhibitor.

In some aspects, provided herein are compositions comprising an inhibitor of C5L2 for use in performing any method involving use of a C5L2 inhibitor, optionally wherein the composition is a pharmaceutical composition.

In some aspects, provided herein is a method of identifying a candidate inhibitor of C5L2, the method comprising contacting a mammalian T cell or monocyte with a test agent and determining whether the test agent increases production of IL-6, IL-1β, or both, by the T cell, wherein an agent that increases production of IL-6, IL-1β, or both, by the T cell or monocyte is a candidate inhibitor of C5L2. In some embodiments the T cell is a CD4+ T cell.

In some embodiments a C5L2 inhibitor enhances secretion of IL-6, IL-1β, or both, by mammalian T cells or monocytes.

In some aspects, described herein is method of identifying a candidate inhibitor of C5L2, the method comprising contacting a mammalian Treg cell, e.g., a mammalian nTreg cell, with a test agent and determining whether the test agent decreases suppressive activity of the nTreg cell, wherein an agent that decreases suppressive activity of the Treg cell, e.g., nTreg cell, is a candidate inhibitor of C5L2.

In some aspects, described herein are compositions comprising an inhibitor of C5L2 for use in performing any of the methods involving use of a C5L2 inhibitor, optionally wherein the composition is a pharmaceutical composition.

In some aspects, described herein is a method of inhibiting production of interleukin-17 (IL-17), interferon gamma (IFN-γ), or both, by a mammalian T cell, e.g., CD4+ T cell, the method comprising contacting the cell with a C5L2 activator.

In some aspects, described herein is a method of inhibiting Th1 and/or Th17 responses by a mammalian T cell, the method comprising contacting the cell with a C5L2 activator. In some embodiments the T cell is a CD4+ T cell. In some embodiments a CD4+ cell is a resting CD4+ T cell. In some embodiments a CD4+ cell is an activated CD4+ T cell.

In some aspects, described herein is a method of inhibiting production of interleukin-6 (IL-6), interleukin 1 beta (IL-1β), or both by a mammalian T cell or monocyte, the method comprising contacting the cell with a C5L2 activator. In some embodiments the T cell is a CD4+ T cell. In some embodiments a CD4+ cell is a resting CD4+ T cell. In some embodiments a CD4+ cell is an activated CD4+ T cell.

In some aspects, described herein is a method of increasing suppressive activity of a mammalian Treg cell, e.g., a mammalian nTreg cell, the method comprising contacting the cell with a C5L2 activator.

In some embodiments of any aspect relating to a C5L2 activator, the C5L2 activator is a C5L2 agonist. In some embodiments of any aspect wherein the C5L2 activator is a C5L2 agonist, the C5L2 agonist is selective for C5L2 receptor versus C5a receptor. In some embodiments of any aspect relating to a C5L2 activator the C5L2 activator comprises an antibody, an engineered non-antibody polypeptide, a peptide, a peptidomimetic, a nucleic acid, or a small molecule. In some embodiments of any aspect relating to a C5L2 activator, the C5L2 activator comprises a variant of C5a, optionally lacking Arg74 of C5a, and further optionally comprising a substitution at position 69 of C5a. In some embodiments of any aspect relating to a C5L2 activator, the C5L2 activator comprises C5adesArg. In some embodiments of any aspect relating to a C5L2 activator, the C5L2 activator comprises an enzyme that processes C5a into C5adesArg or an agent that increases expression or activity of an enzyme that processes C5a into C5adesArg. In some embodiments of any aspect relating to a C5L2 activator the C5L2 activator comprises a carboxypeptidase capable of cleaving C5a to form C5adesArg. In some embodiments the carboxypeptidase comprises a catalytically active variant or fragment of CPM, optionally wherein the catalytically active variant or fragment of CPM lacks at least a sufficient portion of the CPM GPI anchor sequence so that the protein is secreted when expressed by eukaryotic cells.

In some embodiments, contacting a cell with a C5L2 activator comprises contacting the cell with a C5L2 activator in vivo. In some embodiments, contacting a cell with a C5L2 activator in vivo comprises administering the C5L2 activator to a mammalian subject. In some embodiments, contacting a cell with a C5L2 activator in vivo comprises administering the C5L2 activator to a mammalian subject who may benefit from decreased production of IL-17 and/or decreased production of IFN-γ. In some embodiments, contacting a cell with a C5L2 activator in vivo comprises administering the C5L2 activator to a mammalian subject who may benefit from decreased Th1 and/or Th17 responses. In some embodiments a subject who may benefit from decreased Th1 and/or Th17 responses is in need of treatment for an autoimmune disease or inflammatory disease. In some embodiments, contacting a cell with a C5L2 activator in vivo comprises administering the C5L2 activator to a mammalian subject who may benefit from decreased production of IL-6 and/or decreased production of IL-1β, optionally wherein the subject has an IL-6 mediated disease. In some embodiments a subject who may benefit from decreased production of IL-6 and/or decreased production of IL-1β is in need of treatment for an autoimmune disease or inflammatory disease. In some embodiments, contacting a cell with a C5L2 activator in vivo comprises administering the C5L2 activator to a mammalian subject who may benefit from increased nTreg suppressive activity. In some embodiments a subject who may benefit from a increase in suppressive activity of nTreg cells is in need of treatment for an autoimmune disease or inflammatory disease.

In some aspects, provided herein are methods of treating a subject in need of treatment for an IL-6 mediated disease, the methods comprising treating the subject with a C5L2 activator.

In some aspects, described herein is a method of identifying a candidate activator of C5L2, the method comprising contacting a mammalian T cell with a test agent and determining whether the test agent decreases production of IL-17, IFN-γ, or both, by the T cell, wherein an agent that decreases production of IL-17, IFN-γ, or both, by the T cell, is a candidate activator of C5L2. In some embodiments the T cell is a CD4+ T cell.

In some aspects, described herein is a method of method of identifying a candidate activator of C5L2, the method comprising contacting a mammalian T cell with a test agent and determining whether the test agent decreases production of IL-6, IL-1β, or both, by the T cell or monocyte, wherein an agent that decreases production of IL-6, IL-1β, or both, by the T cell or monocyte, is a candidate activator of C5L2. In some embodiments the T cell is a CD4+ T cell.

In some aspects, described herein is method of identifying an inhibitor of Th1 and/or Th17 responses, the method comprising identifying a C5L2 activator. In some embodiments the C5L2 activator inhibits secretion of IL-17, IFN-γ, or both by the mammalian T cell, e.g., a CD4+ T cell. In some embodiments the C5L2 activator inhibits secretion of IL-6, IL-1β, or both by the mammalian T cell or monocyte.

In some embodiments of any aspect relating to a C5L2 inhibitor or C5L2 activator, the C5L2 inhibitor or C5L2 activator is physically associated with a clearance reducing moiety, targeting moiety, a cell uptake moiety, a cell-reactive moiety, or a cell membrane binding moiety. In some embodiments a physical association is via a covalent bond. In some embodiments a physical association is via a non-covalent bond. In some embodiments a physical association is via a linking moiety, which linking moiety may be covalently bonded to the C5L2 inhibitor of C5L2 activator, may be covalently bonded to the clearance reducing moiety, targeting moiety, a cell uptake moiety, a cell-reactive moiety, or a cell membrane binding moiety, or both.

In some aspects, described herein are compositions comprising an activator of C5L2 for use in performing any of the methods involving use of a C5L2 activator, optionally wherein the composition is a pharmaceutical composition.

In some aspects, described herein are agents comprising a C5L2 inhibitor or a C5L2 activator, wherein the C5L2 inhibitor or C5L2 activator is physically associated with a clearance reducing moiety, a targeting moiety, a cell uptake moiety, a cell-reactive moiety, or a cell membrane binding moiety, wherein optionally the C5L2 inhibitor or activator is covalently linked to the clearance reducing moiety, targeting moiety, cell uptake moiety, cell-reactive moiety, or cell membrane binding moiety. In some embodiments the clearance reducing moiety comprises polyethylene glycol or another organic polymer, optionally a synthetic organic polymer. In some embodiments the targeting moiety binds to a cell surface marker of a target cell. In some embodiments the targeting moiety binds to a cell surface marker of a target cell, wherein the target cell is a T cell, a monocyte, a cancer cell, or a pathogen-infected cell. In some embodiments the targeting moiety comprises an antibody, a non-antibody polypeptide, an aptamer, or a small molecule, that binds to a target. In some embodiments the cell uptake moiety comprises a cell penetrating peptide. In some embodiments the cell-reactive moiety comprises a reactive functional group that reacts with a functional group exposed at a cell surface to form a covalent bond. In some embodiments the cell membrane binding moiety comprises at least one lipophilic binding element, optionally comprising one or more comprising aliphatic acyl groups. In some embodiments the cell membrane binding moiety comprises (i) at least one lipophilic binding element, optionally comprising one or more comprising aliphatic acyl groups, and (ii) a hydrophilic peptide, optionally wherein the lipophilic binding element is linked to the hydrophilic peptide.

In some aspects, provided herein are compositions comprising a C5L2 inhibitor and a second agent, wherein the second agent is useful for treatment of cancer or an infection.

In some aspects, provided herein are compositions comprising a C5L2 activator and a second agent, wherein the second agent is useful for treatment of an autoimmune disease or an inflammatory disease.

In some aspects, provided herein are pharmaceutical compositions comprising any of the agents or compositions.

In some aspects, provided herein are methods of treating a subject in need thereof, the methods comprising administering an agent or composition described herein to the subject. In some embodiments the agent or composition comprises a C5L2 inhibitor and the subject is in need of treatment for cancer or an infection. In some embodiments the agent or composition comprises a C5L2 activator and the subject is in need of treatment for an autoimmune disease or inflammatory disease.

In some embodiments of any aspect relating to a mammalian cell, the mammalian cell is a human cell. For example, in some embodiments of any aspect relating to a mammalian T cell, the mammalian T cell is a human T cell; in some embodiments of any aspect relating to a mammalian monocyte, the mammalian monocyte is a human monocyte. In some embodiments of any aspect relating to a mammalian subject, the mammalian subject is a human subject.

In some embodiments, a T cell is contacted with a C5L2 inhibitor in vitro. In some embodiments, a T cell is contacted with a C5L2 activator in vitro. In some embodiments, a cell contacted with a C5L2 activator or C5L2 inhibitor in vitro is to be introduced into a subject, e.g., for therapeutic purposes. In some embodiments the cell is to be introduced into the subject as part of or in conjunction with an organ transplant, bone marrow transplant, blood transfusion, vaccine, or immunotherapy, optionally wherein the vaccine or immunotherapy is for cancer or an infectious disease.

The practice of certain aspects of the present invention may employ conventional techniques of molecular biology, cell culture, recombinant nucleic acid (e.g., DNA) technology, immunology, microbiology, nucleic acid and/or polypeptide synthesis, detection, manipulation, and quantification, and RNA interference that are within the ordinary skill of the art. See, e.g., Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988. Information regarding immunology and the immune system may be found, e.g., in textbooks such as Murphy, K, Janeway's Immunobiology, Garland Science; 8th edition (2011); Paul, W., Fundamental Immunology, 6th ed. Lippincott Williams & Wilkins; 7 Har/Psc edition (2012); Abbas, A, et al., Cellular and Molecular Immunology, Saunders, 7th edition (2011); Information regarding various disorders of interest herein and therapeutic agents useful for treatment of such disorders may be found, e.g., in standard textbooks of internal medicine such as Cecil Textbook of Medicine (e.g., 23rd edition), Harrison's Principles of Internal Medicine (e.g., 17th edition), and/or standard textbooks focusing on particular areas of medicine, particular body systems or organs, and/or particular disorders.

All articles, books, patent applications, patents, other publications, websites, and databases mentioned in this application are incorporated herein by reference. In the event of a conflict between the specification and any of the incorporated references the specification (including any amendments thereto) shall control. Unless otherwise indicated, art-accepted meanings of terms and abbreviations are used herein.

### BRIEF DESCRIPTION OF THE DRAWING

FIG. 1A. Photomicrograph showing resting CD4+ T cells stained for intracellular C5. FIG. 1B. Slide showing location of C3 and C5 in resting and activated CD4+ T cells.

FIG. 2. FACS data showing staining for intracellular C5 as well as C5a (detected via an antibody that only recognizes the C5a neo-epitope and not the C5a portion still contained within the uncleaved C5 alpha-chain) in resting and activated CD4+ T cells. CD4+ T cells were isolated and left either non-activated or were activated with the depicted immobilized antibodies. At 20 h post activation, intracellular C5 and C5a expression was determined by FACS using an intracellular staining protocol. Shown is one representative experiment of three similarly performed (n=3).

FIG. 3A. C5aR expression in resting and activated human CD4+ T cells. CD4+ T cells were isolated and left nonactivated or CD3/CD46-activated. At the depicted time points, extra- and intracellular C5aR expression was determined by FACS. Shown is one representative experiment of four similarly performed (n=4). FIG. 3B. C5L2 expression in resting and activated human CD4+ T cells. CD4+ T cells were isolated and left nonactivated or CD3/CD46-activated. At the depicted time points, extra- and intracellular C5L2 expression was determined by FACS. Shown is one representative experiment of four similarly performed (n=4).

FIG. 4. Summary of data in FIG. 3A and FIG. 3B.

FIG. 5A. Schematic diagram depicting components of the C5 axis. FIG. 5B. Schematic diagram of the receptor blocking activities of the C5aR antagonist and the dual C5aR/C5L2 antagonist.

FIG. 6. Bar graphs showing the effect of C5L2 blockade on secretion of IL-17, IFN-γ, and TNFα by resting and activated CD4+ T cells. Similar bar graphs showing secretion of IL-17, IFN-γ, TNFα, IL-10, and IL-4 by resting and activated CD4+ T cells from C5-deficient patient were also prepared (data not shown).

FIG. 7A. Bar graphs showing the effect of C5L2 blockade on secretion of IL-10, IL-6, and IL-4 by resting and activated CD4+ T cells. FIG. 7B. Bar graphs showing statistical analysis (2 way ANOVA with Bonferroni Post Hoc analysis, n=6).

FIG. 8. Effect of C5L2 blockage on IL-1β secretion by resting and activated CD4+ T cells and by monocytes. Bar graphs show that C5L2 blockade induces IL-1β release by human CD4+ T cells and monocytes. CD4+ T cells were isolated and left non-activated or CD3/CD46-activated as described (with or without addition of the C5aR/C5L2 double antagonist, dRA, 7 μM) and IL-1β secretion into the media measured at 24 h post activation using an ELISA. CD14+ monocytes (2.5×10⁵) were left non-activated or activated with 100 ng/ml LPS for 12 h and supernatants assayed for IL-1β by ELISA. Data represent n=3 experiments with SEM for the CD4+ T cells and n=1 for monocytes (conditions were performed in duplicate and shown are the median values of each condition).

FIG. 9. C5L2 blockade induces pro-inflammatory cytokine release by human monocytes. CD14+ monocytes (2.5×10⁵) were cultured for 36 hours and supernatants assayed for cytokines using CBA. Monocytes were treated with either C5aR/C5L2 double receptor antagonist (dRA; 7 μM), C5aR antagonist (PMX53; 10 μM) or control (non-treated). The effect of antagonist treatment was assessed on non-activated monocytes (white bars) as well as monocytes activated with 1 μg/ml of either Flagellin (light grey bars) or 100 ng/ml LPS (dark grey bars). Data represent n=4 experiments with SEM indicated. Statistical testing was not conducted as insufficient experimental repeats were performed to assume normal distribution. Asterisk denotes that the median value was 10,400 pg/ml.

FIG. 10. Carboxypetidase M expression in resting and activated CD4+ T cells.

FIG. 11. CD4+ T cells express carboxypetidase (CP) M but not CPA or CPB. (A) Resting and activated CD4⁺ T cells express intra- and extracellular CPM. CD4⁺ T cells were isolated and left non-activated or activated with the depicted immobilized antibodies. At 1 h post activation, intracellular and extracellular CPM expression was determined by FACS (rabbit anti-CPM from Abcam, ab136033, 1:100). Shown is one representative experiment of three similarly performed (n=3). (B) and (C) Resting CD4+ T cells do not express CPA (A) or CPB (B). Resting T cells were stained with an intracellular staining protocol for CPA (rabbit anti-CPA, Abcam 115283, 1:100) or CPB (mouse anti-CPB, Abcam 54581, 1:100) and analysed by FACS analysis. Shown are representative data from 2 donors tested (n=2).

FIG. 12A. Effect of carboxypeptidase M (CPM) inhibition on cytokine secretion in resting and activated CD4+ T cells. CD4+ T cells (2.5×10⁵) were activated as depicted for 36 hours and supernatants assayed for Th1/2/17 cytokines using CBA. Cells were treated with either C5aR/C5L2 double receptor antagonist (dRA; 7 μM), C5aR antagonist (PMX53; 10 μM), with or without a carboxypetidase M inhibitor (CPMi) or activated in media without any addition as a first control (non-treated, NT) or with a control peptide that lacks activity towards C5a and C5L2 as a second control (Control). The effect of antagonist treatment was assessed in non-activated T cells (white bars), as well as T cells activated with 0.25 μg plate-bound anti-CD3 (light grey bars), anti-CD3/28 (dark grey bars) or anti-CD3/46 (black bars). Data represent n=4 with standard error of the mean indicated. Bar graphs showing effect of CPMi on secretion of IL-17, IFN-γ, and TNFα by resting and activated CD4+ T cells. FIG. 12B. Effect of carboxypeptidase M (CPM) inhibition on cytokine secretion in resting and activated CD4+ T cells, as in FIG. 12A. Bar graphs showing effect of CPMi on IL-10, IL-6, and IL-4 secretion by resting and activated CD4+ T cells.

FIG. 13. Serum-purified C5adesArg can partially rescue carboxypeptidase M inhibitor (CPM)-mediated increase in IFN-γ production by CD4+ T cells. Purified human CD4+ T cells were activated with depicted immobilized antibodies in media, or in media with the addition of a CPM inhibitor with or without either serum-purified C5a or C5adesArg. IFN-γ production by cells was assessed 24 h post activation using the CBA Cytokine Bead Array (Miltenyi Biotec). Data (±SD) are from three experiments (n=3). *, p<0.05; (student's paired t-test).

FIG. 14. C5L2 blockage decreases suppressive activity of natural regulatory T cells (nTregs). nTregs and effector T cells from a freshly drawn human blood sample were separated by cell sorting (CD4⁺CD25^{hi}CD127^{lo} Treg cells; CD4⁺CD25^{lo}CD127^{hi} effector T cells). nTreg cells were incubated in media containing 7 μM of the C5ar/C5L2 double antagonist (dRA) for 8 hr. Cells were then washed twice to remove the dRA and used for a suppression assay via CSFE dilution measurement in 1:1 co-culture and percentage of suppression calculated. Shown are the mean values of two separately performed experiments.

FIG. 15. Effect of C5L2 blockage on TGF-β secretion in CD4+ T cells.

FIG. 16. C5L2 regulates TGF-β receptor chain expression in CD4+ T cells.

FIG. 17A. Schematic model of autocrine activity of C5 axis in T cells according to certain embodiments. (CP is carboxypeptidase.) FIG. 17B. Schematic model of autocrine activity of C3 and C5 axes in T cells according to certain embodiments.

FIG. 18. Reference sequences of human C5 preprotein, C5a, C5aR, C5L2, and CPM precursor.

### DETAILED DESCRIPTION OF CERTAIN EMBODIMENTS

### I. Glossary

Descriptions and information relating to certain terms used in the present disclosure are collected here for convenience.

“Agent” is used interchangeably with “compound” herein to refer to any substance, compound (e.g., molecule), supramolecular complex, material, or combination or mixture thereof. A compound may be any agent that can be represented by a chemical formula, chemical structure, or sequence. Example of agents, include, e.g., small molecules, polypeptides, nucleic acids (e.g., RNAi agents, antisense oligonucleotide, aptamers), lipids, polysaccharides, etc. In general, agents may be obtained using any suitable method known in the art. The ordinary skilled artisan will select an appropriate method based, e.g., on the nature of the agent. An agent may be at least partly purified. In some embodiments an agent may be provided as part of a composition, which may contain, e.g., a counter-ion, aqueous or non-aqueous diluent or carrier, buffer, preservative, or other ingredient, in addition to the agent, in various embodiments. In some embodiments an agent may be provided as a salt, ester, hydrate, or solvate. In some embodiments an agent is cell-permeable, e.g., within the range of typical agents that are taken up by cells and act intracellularly, e.g., within mammalian cells, to produce a biological effect. Certain compounds may exist in particular geometric or stereoisomeric forms. Such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates (e.g., with water (i.e. hydrates) or common solvents) and/or may have different crystalline forms (e.g., polymorphs) or different tautomeric forms. Embodiments exhibiting such alternative protonation states, configurations, solvates, and forms are encompassed by the present disclosure where applicable.

As used herein, the term “antibody” refers to an immunoglobulin, whether natural or wholly or partially synthetically produced. The term encompasses antibodies and antibody fragments comprising an antigen binding site. An antibody may originate from any of a variety of vertebrate (e.g., mammalian or avian) organisms, e.g., mouse, rat, rabbit, hamster, goat, chicken, human, camelid, shark, etc., or may be encoded at least in part by immunoglobulin gene sequences derived from any of the foregoing organisms. An antibody may be of any of various antibody classes, e.g., the human classes: IgG (e.g., IgG1, IgG2, IgG3, IgG4), IgM, IgA, IgD, and IgE. As used herein, the term “antibody fragment” refers to any of various portions of an antibody that contain less than a complete antibody structure (e.g., less than the complete structure of a conventional antibody composed of two heavy and two light chains). In general, an antibody fragment retains at least a significant portion of the complete antibody's specific binding ability. Examples of antibody fragments include, but are not limited to, Fab, Fab′, and F(ab′)2 fragments. The term “antibody” encompasses single chain variable (scFv), Fv, dsFv, diabody, minibody, Fd fragments, single domain antibodies (e.g., antibodies comprising a single variable domain, e.g., a heavy chain variable domain, e.g., VH or VHH domain), and nanobodies. Bispecific or multispecific antibodies may be used in various embodiments. The heavy and light chain of IgG immunoglobulins (e.g., rodent or human IgGs) contain four framework regions (FR1 through FR4) separated respectively by three complementarity determining regions (CDR1 through CDR3). The CDRs, particularly the CDR3 regions, especially the heavy chain CDR3, are largely responsible for antibody specificity. An antibody may be a chimeric antibody in which, for example, a variable domain of rodent origin or non-human primate origin is fused to a constant domain of human origin, or a “humanized” antibody in which some or all of the complementarity-determining region (CDR) amino acids that constitute an antigen binding site (sometimes along with one or more framework amino acids or regions) are “grafted” from a rodent antibody (e.g., murine antibody) or phage display antibody to a human antibody, thus retaining the specificity of the rodent or phage display antibody. Thus, humanized antibodies may be recombinant proteins in which only the antibody complementarity-determining regions are of non-human origin. It will be appreciated that the alterations to antibody sequence that are involved in the humanization process are generally carried out through techniques at the nucleic acid level, e.g., standard recombinant nucleic acid techniques. In some embodiments only the specificity determining residues (SDRs), the CDR residues that are most crucial in the antibody-ligand interaction, are grafted. The SDRs may be identified, e.g., through use of a database of the three-dimensional structures of the antigen-antibody complexes of known structures or by mutational analysis of the antibody-combining site. In some embodiments an approach is used that involves retention of more CDR residues, namely grafting of so-called “abbreviated” CDRs, the stretches of CDR residues that include all the SDRs. See, e.g., Kashmiri, S V, Methods. 36(1):25-34 (2005), for further discussion of SDR grafting. See, e.g., Almagro J C, Fransson J. Humanization of antibodies. Front Biosci. 13:1619-33 (2008) for review of various methods of obtaining humanized antibodies. It will be understood that “originate from or derived from” refers to the original source of the genetic information specifying an antibody sequence or a portion thereof, which may be different from the species in which an antibody is initially synthesized. For example, “human” domains may be generated in rodents whose genome incorporates human immunoglobulin genes. See, e.g., Vaughan, et al, (1998), Nature Biotechnology, 16: 535-539, e.g., to generate a fully human antibody. An antibody may be polyclonal or monoclonal, though for purposes of the present invention monoclonal antibodies are generally preferred. Standard methods of antibody identification and production known in the art can be used to produce an antibody that binds to a target molecule or complex of interest. In some embodiments an antibody is a monoclonal antibody. Monoclonal antibodies can be identified and/or produced using, e.g., hybridoma technology or recombinant nucleic acid technology in various embodiments. In some embodiments an antibody or portion thereof (e.g., an antigen-binding portion thereof) is selected from a library and/or using a display technique, e.g., a phage or yeast or ribosome display techniques. Monoclonal can be produced recombinantly, in cell culture and, e.g., purified from culture medium. Polyclonal antibodies can be purified from natural sources, e.g., from blood or ascites fluid of an animal that produces the antibody (e.g., following immunization with the molecule or an antigenic fragment thereof). Affinity purification may be used, e.g., protein A/G affinity purification and/or affinity purification using the antigen as an affinity reagent. See, e.g., Kaser, M. and Howard, G., “Making and Using Antibodies: A Practical Handbook” and Sidhu, S., “Phage Display in Biotechnology and Drug Discovery”, CRC Press, Taylor and Francis Group, 2005, for further information. Methods for generating antibody fragments are well known. For example, F(ab′)2 fragments can be generated, for example, through the use of an Immunopure F(ab′)2 Preparation Kit (Pierce) in which the antibodies are digested using immobilized pepsin and purified over an immobilized Protein A column. The digestion conditions (such as temperature and duration) may be optimized by one of ordinary skill in the art to obtain a good yield of F(ab′)2. The yield of F(ab′)2 resulting from the digestion can be monitored by standard protein gel electrophoresis. F(ab′) can be obtained by papain digestion of antibodies, or by reducing the S—S bond in the F(ab′)2. As used herein, a “single-chain Fv” or “scFv” antibody fragment comprises the V_{H} and V_{L} domains of an antibody, wherein these domains are present in a single polypeptide chain. Typically, a scFv antibody further comprises a polypeptide linker between the V_{H} and V_{L} domains, although other linkers could be used to connect the domains in certain embodiments. A linking domain may comprise a peptide of, e.g., about 10 to about 25 amino acids. In some embodiments, an antibody is identified or produced at least in part using recombinant nucleic acid technology (e.g., phage or yeast display). See, e.g., Lonberg N. Fully human antibodies from transgenic mouse and phage display platforms. Curr Opin Immunol. 20(4):450-9, 2008. In some embodiments an antibody is a single polypeptide chain that, in some embodiments, can be expressed intracellularly in functional form. In some embodiments an antibody substantially lacks the capacity to activate complement. For example, the antibody may have less than 10%, less than 5%, or less than 1% complement stimulating activity as compared with full length human IgG1. In some embodiments, the antibody comprises a CH2 domain that has reduced ability to bind C1q as compared with human IgG1 CH2 domain. In some embodiments, the antibody contains CH1, CH2, and/or CH3 domains from human IgG4 and/or does not contain CH1, CH2, and/or CH3 domains from human IgG1.

The terms “approximately” or “about” in reference to a number generally include numbers that fall within ±10%, in some embodiments ±5%, in some embodiments ±1%, in some embodiments ±0.5% of the number unless otherwise stated or otherwise evident from the context (except where such number would impermissibly exceed 100% of a possible value) or other values reasonably close to a given value as would be understood to constitute “approximately” or “about” in a given context by one of ordinary skill in the art.

As used herein, a “chronic disorder” is a disorder that persists for at least 3 months and/or is accepted in the art as being a chronic disorder. In some embodiments, a chronic disorder persists for at least 6 months, e.g., at least 1 year, or more, e.g., indefinitely. One of ordinary skill in the art will appreciate that at least some manifestations of various chronic disorders may be intermittent and/or may wax and wane in severity over time. A chronic disorder may be progressive, e.g., having a tendency to become more severe or affect larger areas over time. In some embodiments, a disorder, e.g., a chronic disorder, is a Th1 disorder. In some embodiments, a disorder, e.g., a chronic disorder, is a Th17 disorder. In some embodiments, a disorder, e.g., a chronic disorder, is an IL6-mediated disorder. In some embodiments a chronic disorder is a chronic infection, cancer, autoimmune disease, or inflammatory disease.

“Linked”, as used herein with respect to two or more moieties, means that the moieties are physically associated or connected with one another to form a molecular structure that is sufficiently stable so that the moieties remain associated under the conditions in which the linkage is formed and, preferably, under the conditions in which the new molecular structure is used, e.g., physiological conditions. In certain preferred embodiments of the invention the linkage is a covalent linkage. In other embodiments the linkage is noncovalent. Moieties may be linked either directly or indirectly. When two moieties are directly linked, they are either covalently bonded to one another or are in sufficiently close proximity such that intermolecular forces between the two moieties maintain their association. When two moieties are indirectly linked, they are each linked either covalently or noncovalently to a third moiety, which maintains the association between the two moieties. In general, when two moieties are referred to as being linked by a “linking moiety” or “linking portion”, the linkage between the two linked moieties is indirect, and typically each of the linked moieties is covalently bonded to the linking moiety. Two moieties may be linked using a “linker”. A linker can be any suitable moiety that reacts with the entities to be linked within a reasonable period of time, under conditions consistent with stability of the entities (portions of which may be protected as appropriate, depending upon the conditions), and in sufficient amount, to produce a reasonable yield. Typically the linker will contain at least two functional groups, one of which reacts with a first entity and the other of which reacts with a second entity. It will be appreciated that after the linker has reacted with the entities to be linked, the term “linker” may refer to the part of the resulting structure that originated from the linker, or at least the portion that does not include the reacted functional groups. A linking moiety may comprise a portion that does not participate in a bond with the entities being linked, and whose main purpose may be to spatially separate the entities from each other. Such portion may be referred to as a “spacer”.

“Nucleic acid” is used interchangeably with “polynucleotide” and encompasses polymers of nucleotides. “Oligonucleotide” refers to a relatively short nucleic acid, e.g., typically between about 4 and about 100 nucleotides (nt) long, e.g., between 8-60 nt or between 10-40 nt long. Nucleotides include, e.g., ribonucleotides or deoxyribonucleotides. In some embodiments a nucleic acid comprises or consists of DNA or RNA. In some embodiments a nucleic acid comprises or includes only standard nucleobases (often referred to as “bases”). The standard bases are cytosine, guanine, adenine (which are found in DNA and RNA), thymine (which is found in DNA) and uracil (which is found in RNA), abbreviated as C, G, A, T, and U, respectively. In some embodiments a nucleic acid may comprise one or more non-standard nucleobases, which may be naturally occurring or non-naturally occurring (i.e., artificial; not found in nature) in various embodiments. In some embodiments a nucleic acid may comprise chemically or biologically modified bases (e.g., alkylated (e.g., methylated) bases), modified sugars (e.g., 2′-O-alkyribose (e.g., 2′-O methylribose), 2′-fluororibose, arabinose, or hexose), modified phosphate groups (e.g., phosphorothioates or 5′-N-phosphoramidite linkages). In some embodiments a nucleic acid comprises subunits (residues), e.g., nucleotides, that are linked by phosphodiester bonds. In some embodiments, at least some subunits of a nucleic acid are linked by a non-phosphodiester bond or other backbone structure. In some embodiments, a nucleic acid comprises a locked nucleic acid, morpholino, or peptide nucleic acid. A nucleic acid may be linear or circular in various embodiments. A nucleic acid may be single-stranded, double-stranded, or partially double-stranded in various embodiments. An at least partially double-stranded nucleic acid may be blunt-ended or may have one or more overhangs, e.g., 5′ and/or 3′ overhang(s). Nucleic acid modifications (e.g., base, sugar, and/or backbone modifications), non-standard nucleotides or nucleosides, etc., such as those known in the art as being useful in the context of RNA interference (RNAi), aptamer, or antisense-based molecules may be incorporated in various embodiments. Such modifications may, for example, increase stability (e.g., by reducing sensitivity to cleavage by nucleases), decrease clearance in vivo, increase cell uptake, or confer other properties that improve the potency, efficacy, specificity, or otherwise render the nucleic acid more suitable for an intended use. Various non-limiting examples of nucleic acid modifications are described in, e.g., Deleavey G F, et al., Chemical modification of siRNA. Curr. Protoc. Nucleic Acid Chem. 2009; 39:16.3.1-16.3.22; Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008; U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; 6,455,308 and/or in PCT application publications WO 00/56746 and WO 01/14398. Different modifications may be used in the two strands of a double-stranded nucleic acid. A nucleic acid may be modified uniformly or on only a portion thereof and/or may contain multiple different modifications.

“Polypeptide”, as used herein, refers to a polymer of amino acids, optionally including one or more amino acid analogs. A protein is a molecule composed of one or more polypeptides. A peptide is a relatively short polypeptide, typically between about 2 and 60-100 amino acids in length. The terms “protein”, “polypeptide”, and “peptide” may be used interchangeably. Polypeptides used herein may contain amino acids such as those that are naturally found in proteins, amino acids that are not naturally found in proteins, and/or amino acid analogs that are not amino acids. As used herein, an “analog” of an amino acid may be a different amino acid that structurally resembles the amino acid or a compound other than an amino acid that structurally resembles the amino acid. A large number of art-recognized analogs of the 20 amino acids commonly found in proteins (the “standard” amino acids) are known. One or more of the amino acids in a polypeptide may be modified, for example, by the addition of a chemical entity such as a carbohydrate group, a phosphate group, a farnesyl group, an isofarnesyl group, a fatty acid group, a linker for conjugation, functionalization, or other modification, etc. In some embodiments a polypeptide comprises only standard amino acids (“standard amino acids” are glycine, leucine, isoleucine, valine, alanine, phenylalanine, tyrosine, tryptophan, aspartic acid, asparagine, glutamic acid, glutamine, cysteine, methionine, arginine, lysine, proline, serine, threonine and histidine). Certain polypeptides may incorporate one or more non-standard amino acids. Useful non-standard amino acids include singly and multiply halogenated (e.g., fluorinated) amino acids, D-amino acids, homo-amino acids, N-alkyl amino acids, dehydroamino acids, aromatic amino acids (other than phenylalanine, tyrosine and tryptophan), ortho-, meta- or para-aminobenzoic acid, phospho-amino acids, methoxylated amino acids, and α,α-disubstituted amino acids. In certain embodiments one or more L-amino acids may be replaced by the corresponding D-amino acid.

In certain embodiments a blocking moiety may be present at the N- or C-terminus of a polypeptide. A blocking moiety may be any moiety that stabilizes a peptide against degradation that might otherwise occur in mammalian (e.g., human or non-human primate) blood or interstitial fluid. For example, a blocking moiety at the N-terminal end of a polypeptide could be any moiety that alters the structure of the N-terminus of a peptide so as to inhibit cleavage of a peptide bond between the N-terminal amino acid of the peptide and the adjacent amino acid. A blocking moiety at the C-terminal end of a polypeptide could be any moiety that alters the structure of the C-terminus of a peptide so as to inhibit cleavage of a peptide bond between the C-terminal amino acid of the peptide and the adjacent amino acid. Any suitable blocking moieties known in the art could be used. In certain embodiments of the invention an N-terminal blocking moiety comprises an acyl group (i.e., the portion of a carboxylic acid that remains following removal of the —OH group). The acyl group typically comprises between 1 and 12 carbons, e.g., between 1 and 6 carbons, e.g., formyl, acetyl, proprionyl, butyryl, isobutyryl, valeryl, isovaleryl, etc. In certain embodiments a C-terminal blocking moiety is a primary or secondary amine (—NH₂ or —NHR¹, wherein R is an organic moiety such as an alkyl group). In certain embodiments a blocking moiety is any moiety that neutralizes or reduces the positive charge that may otherwise be present at the N-terminus at physiological pH. In certain embodiments a blocking moiety is any moiety that neutralizes or reduces the negative charge that may otherwise be present at the C-terminus at physiological pH. In certain embodiments of the invention, a polypeptide is acetylated or amidated at the N-terminus and/or C-terminus, respectively. A polypeptide may be acetylated at the N-terminus, amidated at the C-terminus, and or both acetylated at the N-terminus and amidated at the C-terminus.

In general, polypeptides may be obtained or produced using any suitable method known in the art. For example, polypeptides may be isolated from natural sources, produced in vitro or in vivo using recombinant DNA technology in suitable expression systems (e.g., by recombinant host cells or transgenic non-human animals or plants), synthesized through chemical means such as solid phase peptide synthesis and/or using methods involving chemical ligation of synthesized peptides (see, e.g., Kent, S., J Pept Sci., 9(9):574-93, 2003 and U.S. Pub. No. 20040115774), or a combination of these. One of ordinary skill in the art would readily select appropriate method(s). Peptides may be prepared by various synthetic methods of peptide synthesis known in the art via condensation of amino acid residues, e.g., in accordance with conventional peptide synthesis methods, may be prepared by expression in vitro or in living cells from appropriate nucleic acid sequences encoding them using methods known in the art. For example, peptides may be synthesized using standard solid-phase methodologies. Potentially reactive moieties such as amino and carboxyl groups, reactive functional groups, etc., may be protected and subsequently deprotected using various protecting groups and methodologies known in the art. See, e.g., “Protective Groups in Organic Synthesis”, 3rd ed. Greene, T. W. and Wuts, P. G., Eds., John Wiley & Sons, New York: 1999. Peptides may be purified using standard approaches such as reversed-phase HPLC. Separation of diasteriomeric peptides, if desired, may be performed using known methods such as reversed-phase HPLC. Preparations may be lyophilized, if desired, and subsequently dissolved in a suitable solvent, e.g., water. The pH of the resulting solution may be adjusted, e.g. to physiological pH, using a base such as NaOH.

A polypeptide may comprise a tag, e.g., an epitope tag, which tag may facilitate purification and/or detection of the polypeptide. Exemplary tags include, e.g., 6×His, HA, Myc, SNUT, FLAG, TAP, etc. In some embodiments, a tag is cleavable, e.g., the tag comprises a recognition site for cleavage by a protease, or is separated from a portion complement inhibiting portion of the polypeptide by a linking portion that comprises a recognition site for cleavage by a protease. For example, a TEV protease cleavage site can be used.

“Recombinant host cells”, “host cells”, and other such terms, denote prokaryotic or eukaryotic cells or cell lines that contain an exogenous nucleic acid (typically DNA) such as an expression vector comprising a nucleic acid that encodes a polypeptide of interest. It will be understood that such terms include the descendants of the original cell(s) into which the vector or other nucleic acid has been introduced. Appropriate host cells include any of those routinely used in the art for expressing polynucleotides (e.g., for purposes of producing polypeptide(s) encoded by such polynucleotides) including, for example, prokaryotes, such as E. coli; and eukaryotes, including for example, fungi, such as yeast (e.g., Pichia pastoris); insect cells (e.g., Sf9), plant cells, and animal cells, e.g., mammalian cells such as CHO, R1.1, B-W, L-M, African Green Monkey Kidney cells (e.g. COS-1, COS-7, BSC-1, BSC-40 and BMT-10) and cultured human cells. The exogenous nucleic acid may be stably maintained as an episome such as a plasmid or may at least in part be integrated into the host cell's genome, optionally after being copied or reverse transcribed. Terms such as “host cells”, etc., are also used to refer to cells or cell lines that can be used as recipients for an exogenous nucleic acid, prior to introduction of the nucleic acid. A “recombinant polynucleotide” is a polynucleotide that contains nucleic acid sequences that are not found joined directly to one another in nature. For example, the nucleic acid sequences may occur in different genes or different species or one or more of the sequence(s) may be a variant of a naturally occurring sequence or may at least in part be an artificial sequence that is not homologous to a naturally occurring sequence. A “recombinant polypeptide” is a polypeptide that is produced by transcription and translation of an exogenous nucleic acid by a recombinant host cell or by a cell-free in vitro expression system and/or that contains amino acid sequences that are not found joined directly to one another in nature. In the latter case, the recombinant polypeptide may be referred to as a “chimeric polypeptide”. The amino acid sequences in a chimeric polypeptide may, for example, occur in different genes or in different species or one or more of the sequence(s) may be a variant of a naturally occurring sequence or may at least in part be an artificial sequence that is not homologous to a naturally occurring sequence. It will be understood that a chimeric polypeptide may comprise two or more polypeptides. For example, first and second polypeptides A and B of a chimeric polypeptide may be directly linked (A-B or B-A) or may be separated by a third polypeptide portion C (A-C-B or B-C-A). In some embodiments, portion C represents a polypeptide linker which may, for example, comprise one or more glycine and/or serine residues, e.g., between 2 and about 20 residues, e.g., GS, GGS, GGGS, GGGGS, or multimers or concatamers of any of the foregoing (or the reverse sequences) in any order. In some embodiments, two or more polypeptides may be linked by non-polypeptide linker(s).

“Reactive functional groups” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds, N-hydroxysuccinimide esters, maleimides, sulfhydryls, and the like. Methods to prepare each of these functional groups are well known in the art and their application to or modification for a particular purpose is within the ability of one of skill in the art (see, for example, Sandler and Karo, eds. ORGANIC FUNCTIONAL GROUP PREPARATIONS, Academic Press, San Diego, 1989, and Hermanson, G., Bioconjugate Techniques, 2^{nd} ed., Academic Press, San Diego, 2008).

A “small molecule” as used herein, is an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

“Specific binding” generally refers to a physical association between a target polypeptide (or, more generally, a target molecule) and a binding molecule such as an antibody or ligand. The association is typically dependent upon the presence of a particular structural feature of the target such as an antigenic determinant, epitope, binding pocket or cleft, recognized by the binding molecule. For example, if an antibody is specific for epitope A, the presence of a polypeptide containing epitope A or the presence of free unlabeled A in a reaction containing both free labeled A and the binding molecule that binds thereto, will reduce the amount of labeled A that binds to the binding molecule. It is to be understood that specificity need not be absolute but generally refers to the context in which the binding occurs. For example, it is well known in the art that numerous antibodies cross-react with other epitopes in addition to those present in the target molecule. Such cross-reactivity may be acceptable depending upon the application for which the antibody is to be used. One of ordinary skill in the art will be able to select antibodies or ligands having a sufficient degree of specificity to perform appropriately in any given application (e.g., for detection of a target molecule, for therapeutic purposes, etc). It is also to be understood that specificity may be evaluated in the context of additional factors such as the affinity of the binding molecule for the target versus the affinity of the binding molecule for other targets, e.g., competitors. If a binding molecule exhibits a high affinity for a target molecule that it is desired to detect and low affinity for nontarget molecules, the antibody will likely be an acceptable reagent. Once the specificity of a binding molecule is established in one or more contexts, it may be employed in other, preferably similar, contexts without necessarily re-evaluating its specificity. In some embodiments, the affinity (as measured by the equilibrium dissociation constant, Kd) of two molecules, e.g., two molecules that exhibit specific binding, is 10⁻³ M or less, e.g., 10⁻⁴ M or less, e.g., 10⁻⁵ M or less, e.g., 10⁻⁶ M or less, 10⁻⁷ M or less, 10⁻⁸ M or less, or 10⁻⁹ M or less under the conditions tested, e.g., under physiological conditions (e.g., conditions such as salt concentration, pH, and/or temperature, etc., that reasonably approximate corresponding conditions in vivo), or other conditions of the assay. Binding affinity can be measured using any of a variety of methods known in the art. For example, assays based on isothermal titration calorimetry or surface plasmon resonance (e.g., Biacore® assays) can be used in certain embodiments.

A “subject” treated according to the instant invention is typically a human, a non-human primate, or another mammal (e.g., a mouse or rat). In certain embodiments a subject may be a non-human animal that has been genetically engineered to express one or more human complement component(s). In some embodiments the subject is male. In some embodiments the subject is female. In some embodiments a subject is an adult, e.g., a human at least 18 years of age, e.g., between 18 and 100 years of age. In some embodiments a subject is at least 40, 45, 50, 55, 60, 65, 70, 75, or 80 years of age.

“Treating”, as used herein in regard to treating a subject, refers to providing treatment, i.e, providing any type of medical or surgical management of a subject. The treatment can be provided in order to reverse, alleviate, inhibit the progression of, prevent or reduce the likelihood of a disease, or in order to reverse, alleviate, inhibit or prevent the progression of, prevent or reduce the likelihood of one or more symptoms or manifestations of a disease. “Prevent” refers to causing a disease or symptom or manifestation of a disease not to occur for at least a period of time in at least some individuals, e.g., individuals at risk of developing the disease, symptom, or manifestation. Treating can include administering a compound or composition to the subject following the development of one or more symptoms or manifestations indicative of a disease, e.g., in order to reverse, alleviate, reduce the severity of, and/or inhibit or prevent the progression of the disease and/or to reverse, alleviate, reduce the severity of, and/or inhibit or one or more symptoms or manifestations of the disease. A compound or composition can be administered to a subject who has developed a disease, or is at increased risk of developing the disease relative to a member of the general population, optionally a member who is matched with the subject in terms of age, sex, and/or other demographic variable(s). The term “disease” is used interchangeably with “disorder” herein. Certain disorders are sometimes termed a “syndrome” in the art and may be so referred to herein.

A “variant” of a particular polypeptide or polynucleotide has one or more alterations (e.g., additions, substitutions, and/or deletions, which may be referred to collectively as “mutations”) with respect to the polypeptide or nucleic acid, which may be referred to as the “original polypeptide” or “original polynucleotide”, respectively. Thus a variant can be shorter or longer than the polypeptide or polynucleotide of which it is a variant. The terms “variant” encompasses “fragments”. A “fragment” is a continuous portion of a polypeptide that is shorter than the original polypeptide. In certain embodiments of the invention a variant polypeptide has significant sequence identity to the original polypeptide over a continuous portion of the variant that comprises at least 50%, preferably at least 60%, at least 70%, at least 80%, at least 90%, at least 95%, or more, of the length of the variant or the length of the polypeptide, (whichever is shorter). In certain embodiments of the invention a variant polypeptide has substantial sequence identity to the original polypeptide over a continuous portion of the variant that comprises at least 50%, preferably at least 60%, at least 70%, at least 80%, at least 90%, at least 95%, or more, of the length of the variant or the length of the polypeptide, (whichever is shorter). In a non-limiting embodiment a variant has at least 80% identity to the original sequence over a continuous portion of the variant that comprises between 90% and 100% of the variant, e.g., over 100% of the length of the variant or the length of the polypeptide, (whichever is shorter). In another non-limiting embodiment a variant has at least 80% identity to the original sequence over a continuous portion of the variant that comprises between 90% and 100% of the variant, e.g., over 100% of the length of the variant or the length of the polypeptide, (whichever is shorter). In specific embodiments the sequence of a variant polypeptide has N amino acid differences with respect to an original sequence, wherein N is any integer between 1 and 10. In other specific embodiments the sequence of a variant polypeptide has N amino acid differences with respect to an original sequence, wherein N is any integer between 1 and 20. An amino acid “difference” refers to a substitution, insertion, or deletion of an amino acid. In some embodiments an alteration, e.g., a substitution or deletion, e.g., in a functional variant, does not alter or delete an amino acid or nucleotide that is known or predicted to be important for an activity, e.g., a known or predicted catalytic residue or residue involved in binding a substrate or cofactor. In some embodiments nucleotide(s), amino acid(s), or region(s) exhibiting lower degrees of conservation across species as compared with other amino acids or regions may be selected for alteration. Variants may be tested in one or more suitable assays to assess activity.

In certain embodiments a fragment or variant possesses sufficient structural similarity to the original polypeptide so that when its 3-dimensional structure (either actual or predicted structure) is superimposed on the structure of the original polypeptide, the volume of overlap is at least 70%, preferably at least 80%, more preferably at least 90% of the total volume of the structure of the original polypeptide. A partial or complete 3-dimensional structure of the fragment or variant may be determined by crystallizing the protein, which can be done using standard methods. Alternately, an NMR solution structure can be generated, also using standard methods. A modeling program such as MODELER (Sali, A. and Blundell, T L, J. Mol. Biol., 234, 779-815, 1993), or any other modeling program, can be used to generate a predicted structure. If a structure or predicted structure of a related polypeptide is available, the model can be based on that structure. The PROSPECT-PSPP suite of programs can be used (Guo, J T, et al., Nucleic Acids Res. 32(Web Server issue):W522-5, Jul. 1, 2004).

In some embodiments the sequence of a variant polypeptide comprises or consists of a sequence that has N amino acid differences with respect to an original sequence, wherein N is any integer between 1 and 10 or between 1 and 20 or any integer up to 1%, 2%, 5%, or 10% of the number of amino acids in the original polypeptide, where an “amino acid difference” refers to a substitution, insertion, or deletion of an amino acid. In some embodiments a difference is a conservative substitution. Conservative substitutions may be made, e.g., on the basis of similarity in side chain size, polarity, charge, solubility, hydrophobicity, hydrophilicity and/or the amphipathic nature of the residues involved. In some embodiments, conservative substitutions may be made according to Table A, wherein amino acids in the same block in the second column and in the same line in the third column may be substituted for one another other in a conservative substitution. Certain conservative substitutions are substituting an amino acid in one row of the third column corresponding to a block in the second column with an amino acid from another row of the third column within the same block in the second column.

TABLE A

| | | |
|---|---|---|
| Aliphatic | Non-polar | G A P |
| | | I L V |
| | Polar - uncharged | C S T M |
| | | N Q |
| | Polar - charged | D E |
| | | K R |
| Aromatic | | H F W Y |

In certain embodiments one, more than one, or all biological functions or activities of a variant or fragment is substantially similar to that of the corresponding biological function or activity of the original molecule. In certain embodiments the activity of a variant or fragment may be at least 20%, at least 50%, at least 60%, at least 70%, at least 80%, or at least 90% of the activity of the original molecule, up to approximately 100%, approximately 125%, or approximately 150% of the activity of the original molecule. In certain embodiments an activity of a variant or fragment is such that the amount or concentration of the variant needed to produce an effect is within 0.5 to 5-fold of the amount or concentration of the original molecule needed to produce that effect. The invention contemplates use of variants of any of the polypeptides disclosed herein, wherein the variant has sufficient activity to be useful in a method described herein. In some embodiments, a variant lacks or has a substantially reduction in a property that may be undesired such as immunogenicity.

A “vector” may be any of a number of nucleic acid molecules or viruses or portions thereof that are capable of mediating entry of, e.g., transferring, transporting, etc., a nucleic acid of interest between different genetic environments or into a cell. The nucleic acid of interest may be linked to, e.g., inserted into, the vector using, e.g., restriction and ligation. Vectors include, for example, DNA or RNA plasmids, cosmids, naturally occurring or modified viral genomes or portions thereof, nucleic acids that can be packaged into viral capsids, mini-chromosomes, artificial chromosomes, etc.

As used herein, “L-amino acid” refers to any of the naturally occurring levorotatory alpha-amino acids normally present in proteins or the alkyl esters of those alpha-amino acids. The term “D-amino acid” refers to dextrorotatory alpha-amino acids. Unless specified otherwise, all amino acids referred to herein are L-amino acids.

### II. The C5 Axis in T Cells and Monocytes

It has previously been shown that the C3 complement activation fragments C3a and C3b, generated by CD4+ T cells upon TCR activation, are required for human Th1 responses via engagement of their respective receptors, C3a receptor (C3aR) and the complement regulator CD46. This observation is underpinned by the fact that CD46-deficient patients throughout life or C3-deficient patients in early childhood suffer from recurrent infections and cannot generate Th1 responses in vitro or in vivo, while their Th2 responses are normal.

In some aspects, the present disclosure relates to the observation that resting and activated T cells store and secrete the anaphylatoxin C5a. C5a is a 74 amino acid polypeptide that acts as a multifunctional proinflammatory mediator. Among other things, C5a causes neutrophil chemoattraction and stimulation, mast cell degranulation, increases vascular permeability, and stimulates cytokine secretion. The C-terminal Arg of C5a is rapidly cleaved in vivo to form C5adesArg, which is much more stable in blood and plasma and has a different spectrum of activities than does C5a. The human C5a receptor, C5aR (also known as CD88) is a member of the seven α-helical transmembrane G protein-coupled receptor (GPCR) family. C5aR contains acidic and tyrosine residues in its N terminal region that interact with the core of C5a and a hydrophobic pocket formed by the transmembrane helices that interacts with residues in the C terminus of C5a. C5aR has high affinity for C5a, with a considerably lower affinity for C5adesArg. The alternative C5a receptor, C5L2 (also known as CSAR2 (complement component 5a receptor 2), GPF77, and GPR77) is a member of the G protein-coupled receptor family. It has about 40% sequence identity with C5aR and a similar enrichment for acidic and tyrosine residues in the N terminal region. Many of the charged and hydrophobic residues in the loops and transmembrane regions of C5aR that are involved in the interaction with the C terminus of C5a are conserved in C5L2. C5L2 has similar affinities for C5a and C5adesArg. C3adesArg binding to C5L2 has been reported in some studies, while other studies have failed to detect such binding. C5aR and C5L2 are expressed by a number of different immune cell types including neutrophils, immature dendritic cells, mast cells, and macrophages, as well as on various other cell types. Signaling via C5a binding to C5aR is believed to occur via mechanisms typical of classical GPCRs involving association with intracellular G proteins. However, unlike classical GPCRs, C5L2 is not known to associate with intracellular G-proteins. Results of various studies by others have suggested that C5L2 may act as a decoy receptor by binding to C5a and preventing it from interacting with C5aR and/or may modulate signal transduction through the beta-arrestin pathway (Okinaga, S., et al., Biochemistry (2003) 42(31):9406-15; Lee, H., et al., Immunol Cell Biol. (2008) 86(2):153-60; Bamberg, C E, et al., J Biol Chem. (2010) 285(10):7633-44.)

The existence of T cell-derived C5a and its role in human T effector cell regulation are hitherto unknown and unexplored. Some embodiments of the present invention define the role of the T cell-produced anaphylatoxin C5a in the induction or regulation of human Th1 responses. Some embodiments of the present invention define the role of the T cell-produced anaphylatoxin C5a in the induction or regulation of human Th17 responses. As described further in the Examples, effector T cell induction in serum free conditions was assessed in CD4+ T cells isolated from healthy donors and a C5-deficient patient. CD4+ T cells from healthy donors were also analyzed in the presence of a C5aR antagonist or a C5aR/C5L2 receptor double antagonist. T cells from the C5-deficient patient and T cells from healthy donors treated with the C5aR/C5L2 double antagonist, but not with the C5aR antagonist, presented with deregulated Th1 and Th17 responses, characterized by significantly increased IFN-γ and IL-17 production. Th2 responses remained unaltered. This finding suggested a role for C5L2 and C5adesArg in Th1 biology. In line with this observation, Applicants found that resting and activated T cells expressed intra- and extracellular C5L2. Further, Applicants identified the T cell-expressed enzyme that processes C5a into C5adesArg. Inhibition of this enzyme during T cell activation also enhanced Th1/Th17 responses. Further, Applicants identified the first known signaling targets downstream of C5L2, the role of which are currently being assessed in C5−/− and C5L2−/− mouse models. Among other things, Applicants' results suggest a division of labor between the anaphylatoxins in T cell regulation. While C3aR signaling drives Th1 activation, C5L2 engagement mediates contraction of Th1 and Th17 responses. These findings add to the emerging concept that complement actively controls the induction, maintenance and contraction of human T cell responses.

In some aspects, the disclosure relates to Applicant's discovery that the C5 axis plays a number of important roles in T cell and monocyte biology and function. Applicants discovered that C5 and C5a are present in resting and activated CD4+ T cells, indicating that intracellular C5 activation occurs in these cells. The alternative C5a receptor, C5L2, was also found to be present on the surface and inside resting and activated T cells. In some aspects, the discovery that intracellular C5 activation occurs in resting and activated CD4+ T cells together with the discovery that C5L2 is present inside cells implies that C5L2 may be activated in these cells, e.g., C5L2 signaling may occur intracellularly in these cells. In some aspects, the present disclosure relates to modulating the C5 axis or a component thereof in T cells and/or monocytes. In some aspects, the present disclosure relates to modulating C5L2-mediated signaling. In some embodiments the disclosure relates to modulating intracellular C5 cleavage and/or modulating production or activity of intracellularly produced C5 cleavage fragments. In some embodiments the disclosure relates to modulating activity of C5L2. In some aspects, the disclosure provides methods of modulating intracellular activity of C5L2. In some embodiments the methods comprise contacting a cell, e.g., a T cell or monocyte, with a cell-permeable C5L2 modulator.

Some aspects of the disclosure relate to the discovery that C5L2 is a negative regulator of Th1 and Th17 responses. For example, as mentioned above, blockade of C5L2 during effector T cell induction was found to lead to significantly increased IFN-gamma and IL-17 production. CD4+ T cells exposed to a dual antagonist that blocked C5aR and C5L2 secreted significantly greater amounts of these cytokines than did control cells or cells exposed to an antagonist that blocked C5aR but not C5L2. Some aspects of the disclosure relate to the discovery that C5L2 is a negative regulator of IL-6. Some aspects of the disclosure relate to the discovery that C5L2 is a negative regulator of IL-1β. For example, blockade of C5L2 was found to induce high, spontaneous IL-6 and IL-1β secretion in resting human CD4+ T cells and in monocytes. Comparable data were obtained with T cells from a C5-deficient patient. Cells from this patient cannot secrete C5a/C5adesArg as they have intracellular C5 but a defect in the secretion of C5 or its activation fragments.

Some aspects of the disclosure relate to the discovery that carboxypeptidase M (CPM) is expressed by resting and activated CD4+ T cells and monocytes and is responsible for autocrine production of C5adesArg by these cells. Comparable results to those obtained using the dual C5aR/C5L2 antagonist were obtained by using T cells activated in the presence of a CPM inhibitor. The latter cells cannot generate significant levels of autocrine C5adesArg via CPM and thus are expected to have reduced C5L2 activity. Importantly, it was found that Th1 responses induced by the CPM inhibitor can be ‘rescued’ to about 25% by addition of C5adesArg but not by addition of C5a, thus demonstrating that activation of C5L2, e.g., through application or administration of C5L2 activators, can be used to inhibit cellular responses.

Some aspects of the disclosure relate to the discovery that C5L2 is required for normal nTreg function. For example, blockade of C5L2 using a dual C5a/C5L2 antagonist was found to reduce the activity of nTregs.

Applicant's results described herein establish, among other things, that C5L2 (i) is an active signaling receptor and mediates homeostatic control over IL-6 and IL-1β in resting CD4+ T cells and monocytes; (ii) aids in the negative control of Th1 and Th17 responses and (iii) plays an important role in normal nTreg function.

In some aspects, the present disclosure relates to one or more T cell subsets. T cells may be broadly divided into helper (Th) T cells and cytotoxic T cells. Helper T cells “help” cytotoxic T cells, B cells, and macrophages by, e.g., secreting cytokines that have various stimulatory effects. Th help can, for example, enhance proliferation and activation of cytotoxic T cells, stimulate B cell proliferation and maturation and antibody production. Helper T cells are typically characterized by expression of the cell surface marker CD4, while cytotoxic T cells express the cell surface marker CD8. Upon exposure to appropriate stimuli resting CD4+ T cells may be stimulated to expand and differentiate into effector Th cells, which carry out various activities such as those mentioned above. Such stimulation may occur in vivo or in vitro (e.g., by exposure to antigen, appropriate co-stimulatory molecules, cytokines, antibodies to the T cell receptor, etc.). Several distinct subsets of effector Th cells have been identified, including Th1 cells, Th2 cells, and Th17 cells. In some aspects, defining characteristics of a Th cell subset (e.g., Th1 cells, Th2 cells, Th17 cells) may include cytokines that they produce, transcription factors that they express, and/or epigenetic modifications in cytokine gene loci. For example, characteristic cytokines produced by the major CD4+ Th cell subsets are IFN-γ for Th1 cells; IL-4, IL-5, and IL-13 for Th2 cells; and IL-17, Il-21, and IL-22 for Th17 cells. Characteristic transcription factors expressed by Th1 cells are STAT1 and T-bet. Characteristic transcription factors expressed by Th2 cells are STAT6 and GATA-3. Characteristic transcription factors expressed by Th17 cells are RORγ1 and STAT3. As will be appreciated, T cell subsets may also differ in their expression of various adhesion molecules, cytokine and chemokine receptors, microRNAs, etc., which differences may be used to distinguish them Development of the various T cell subsets can be promoted by (driven by) different cytokines, which induce or activate TFs that in turn increase expression of the cytokines and other molecules characteristic of that subset. For example, differentiation to Th1 cells can be driven by IL-12 and IFN-γ, which activate T-bet, STAT1, and STAT4. STAT1 induces expression of T-bet, which in turn promotes expression of IFN-γ. Th2 differentiation can be driven by IL-4. Differentiation to Th17 cells can, for example, be driven by IL-6, IL-1β, TGF-β, and sustained by IL-23. In some aspects, a T cell subset may be characterized by one or more particular epigenetic modifications. Histone modifications and DNA modifications are among the important epigenetic modifications. For example, histone proteins (often their N-terminal tails) can be covalently modified through post-translational modifications such as acetylation, methylation, phosphorylation. Histone modifications are thought to affect gene expression by, for example, relaxing or condensing the chromatin structure to activate or repress transcription, respectively. For example, trimethylation of H3K4 (H3K4me3) is associated with gene activation, whereas trimethylation of H3K27 (H3K27me3) serves to repress gene expression. In some aspects, cytokine genes characteristically expressed by a particular subset may have permissive (H3K4me3) marks in cells of that subset (e.g., IFNγ in Th1 cells or IL-4 in Th2 cells), and/or cytokine genes characteristically expressed by a different subset may have repressive (H3K27me3) marks.

Regulatory T cells (CD4(+)CD25(hi)CD127(lo)FOXP3(+) T cells, “Tregs”) are a population of lymphocytes involved in the maintenance of self-tolerance, among other things. Tregs suppress immune responses at a number of levels including induction of T cell activation and T cell effector functions. Major cytokines secreted by Tregs and involved in their suppressive activity include IL-10 and TGF-β. Abnormalities in function or number of Tregs are a feature of autoimmune diseases in humans. FOXP3 is a characteristic transcription factor expressed by Treg. In certain embodiments Tregs are CD4(+)CD25(hi)CD127(lo)FOXP3(+). In certain embodiments nTregs may be isolated based on a CD4(+)CD25(hi)CD127(lo) cell surface marker profile. Other markers expressed by nTregs include cytotoxic T lymphocyte antigen-4 (CTLA-4) and glucocorticoid-induced tumor necrosis factor receptor superfamily member number 18 (GITR). Natural Tregs (nTregs) refer to Tregs that are believed to arise as a distinct lineage in the thymus. The terms Treg and nTreg are used interchangeably herein; however, certain embodiments relate specifically to nTregs.

Some aspects of the disclosure relate to modulation of C5L2 level and/or activity, e.g., so as modulate Th1 and/or Th17 responses, modulate production of IFN-β and/or IL-17, modulate production of IL-6 and/or IL-1β, and/or modulate activity of nTregs. In some embodiments C5L2 level or activity is modulated by contacting one or more cells, e.g., one or more CD4+ T cells or monocytes with a C5L2 modulator. In some embodiments a C5L2 modulator is a C5L2 activator. In some embodiments a C5L2 modulator is a C5L2 inhibitor.

A “Th1 response” refers to an increase in the number of Th1 cells and/or an increase in the level of at least one functional activity of Th1 cells. In some aspects, a Th1 response is characterized by an increase in production of one or more cytokines that are characteristically produced by Th1 cells, such as IFN-γ, by a T cell or population of T cells. “Enhancing a Th1 response” refers to causing or contributing to increased generation of Th1 cells from resting T cells, increased maintenance of Th1 cells in a Th1 state (stabilization), or both, and/or causing or contributing to an increase in at least one functional activity of Th1 cells, such as increased production (e.g., by Th1 cells) of one or more cytokines that are characteristically produced by Th1 cells. Increased generation of Th1 cells may comprise, for example, increased expression or activity of one or more gene products that promote differentiation of resting T cells to effector Th1 cells, decreased expression or activity of one or more gene products that inhibit differentiation of resting T cells to effector Th1 cells, increased proliferation of Th1 cells or cells committed to become Th1 cells, or a combination thereof. Increased maintenance of Th1 cells in a Th1 cell state may comprise inhibiting a gene product or biological process involved in Th1 cell shutdown and/or rendering a Th1 cell less responsive to one or more stimuli that may otherwise induce or contribute to Th1 shutdown. Certain aspects described herein comprise enhancing (increasing, promoting) a Th1 response. “Inhibiting a Th1 response” refers to causing or contributing to decreased generation of Th1 cells from resting T cells, decreased maintenance of Th1 cells in a Th1 state, or both, and/or causing or contributing to an decrease in at least one functional activity of Th1 cells, such as decreased production (e.g., by Th1 cells) of one or more cytokines that are characteristically produced by Th1 cells. Decreased generation of Th1 cells may comprise, for example, decreased expression or activity of one or more gene products that promote differentiation of resting T cells to effector Th1 cells, increased expression or activity of one or more gene products that inhibit differentiation of resting T cells to effector Th1 cells, decreased proliferation of Th1 cells or cells committed to become Th1 cells, or a combination thereof. Decreased maintenance of Th1 cells in a Th1 cell state may comprise increasing the level or activity of a gene product or biological process involved in Th1 cell shutdown and/or rendering a Th1 cell more responsive to one or more stimuli that induce or contribute to Th1 shutdown. Certain aspects described herein comprise inhibiting (reducing, decreasing, suppressing) a Th1 response.

A “Th17 response” refers to an increase in the number of Th17 cells and/or an increase in the level of at least one functional activity of Th17 cells. In some aspects, a Th17 response is characterized by an increase in production of one or more cytokines that are characteristically produced by Th17 cells, such as IL-17 by a T cell or population of T cells. “Enhancing a Th17 response” refers to causing or contributing to increased generation of Th17 cells from resting T cells, increased maintenance of Th17 cells in a Th17 state (stabilization), or both, and/or causing or contributing to an increase in at least one functional activity of Th17 cells, such as increased production (e.g., by Th17 cells) of one or more cytokines that are characteristically produced by Th17 cells. Increased generation of Th17 cells may comprise, for example, increased expression or activity of one or more gene products that promote differentiation of resting T cells to effector Th17 cells, decreased expression or activity of one or more gene products that inhibit differentiation of resting T cells to effector Th17 cells, increased proliferation of Th17 cells or cells committed to become Th17 cells, or a combination thereof. Increased maintenance of Th17 cells in a Th17 cell state may comprise inhibiting a gene product or biological process involved in Th17 cell shutdown and/or rendering a Th17 cell less responsive to one or more stimuli that may otherwise induce or contribute to Th17 shutdown. In some aspects, Th17 cells and Th17 responses may play a role in defending the body against infections and/or cancer. Certain aspects described herein comprise enhancing (increasing, promoting) a Th17 response. “Inhibiting a Th17 response” refers to causing or contributing to decreased generation of Th17 cells from resting T cells, decreased maintenance of Th17 cells in a Th17 state, or both, and/or causing or contributing to an decrease in at least one functional activity of Th17 cells, such as decreased production (e.g., by Th17 cells) of one or more cytokines that are characteristically produced by Th17 cells. Decreased generation of Th17 cells may comprise, for example, decreased expression or activity of one or more gene products that promote differentiation of resting T cells to effector Th17 cells, increased expression or activity of one or more gene products that inhibit differentiation of resting T cells to effector Th17 cells, decreased proliferation of Th17 cells or cells committed to become Th17 cells, or a combination thereof. Decreased maintenance of Th17 cells in a Th17 cell state may comprise increasing the level or activity of a gene product or biological process involved in Th17 cell shutdown and/or rendering a Th17 cell more responsive to one or more stimuli that induce or contribute to Th17 shutdown. Th17 responses and Th17 cells are increasingly recognized as major causative factors of a wide variety of autoimmune and inflammatory diseases. Certain aspects described herein comprise inhibiting (reducing, decreasing, suppressing) a Th17 response.

“Production” of a cytokine refers to increased synthesis of the cytokine, increased secretion (release from the cell) of the cytokine, or both. “Shutdown” of a cell, e.g., a T cell, refers to significant reduction or cessation of functional activities characteristic of that cell (e.g., functional activities that distinguish such T cell from T cells of other subsets), such as production of characteristic cytokines, which may be accompanied by alteration in expression of transcription factors and/or alteration in epigenetic features characteristic of that cell.

A “C5L2 activator” refers to an agent that increases the level of C5L2 in and/or on cells and/or that increases the activity of C5L2 on a molar basis. Increasing the activity of a protein (e.g., C5L2) on a molar basis refers to increasing the activity per mole of protein. In some embodiments a C5L2 activator increases the transcription, stability, or translation of RNA that encodes C5L2. In some embodiments a C5L2 activator is a C5L2 agonist, i.e., the C5L2 activator increases C5L2 activity by binding to C5L2, thereby triggering C5L2 activity. C5a and C5adesArg are naturally occurring agonists of C5L2. In some embodiments an agonist is any agent that mimics the action of C5a and/or C5adesArg on C5L2. In some embodiments a C5L2 activator is an indirect enhancer of C5L2 activity in that it does not physically interact with C5L2 but instead physically interacts with a second protein so as to cause increased C5L2 activity. For example, in some embodiments a C5L2 activator comprises at least a biologically active portion of an enzyme that is capable of cleaving a protein to generate an endogenous C5L2 agonist such as C5a or C5adesArg. In some aspects, a C5L2 activator is an agent that increases C5L2-mediated signaling. In some embodiments a C5L2 activator is an agent that increases C5L2-mediated signaling by physically interacting with C5L2.

A “C5L2 inhibitor” refers to an agent that decreases the level of C5L2 in and/or on cells and/or that decreases the activity of C5L2 on a molar basis. Decreasing the activity of a protein (e.g., C5L2) on a molar basis refers to decreasing the activity per mole of protein. In some embodiments a C5L2 inhibitor decreases the transcription, stability, or translation of RNA that encodes C5L2. In some embodiments a C5L2 inhibitor is a C5L2 antagonist, i.e., the C5L2 activator decreases C5L2 activity by binding to C5L2, thereby inhibiting C5L2 activity by blocking access of or interaction with an endogenous ligand or interacting molecule. In some embodiments a C5L2 inhibitor is an indirect inhibitor of C5L2 activity in that it does not physically interact with C5L2 but instead physically interacts with a second protein so as to cause decreased C5L2 activity. For example, in some embodiments a C5L2 inhibitor inhibits an endogenous enzyme that is capable of cleaving a protein to generate an endogenous C5L2 agonist such as C5a or C5adesArg. In some aspects, a C5L2 inhibitor is an agent that decreases C5L2-mediated signaling. In some embodiments a C5L2 inhibitor is an agent that decreases C5L2-mediated signaling by physically interacting with C5L2.

In some embodiments a C5L2 activator may be used (e.g., contacted with T cells and/or monocytes ex vivo or in vivo) to reduce or at least in part prevent an IL-17 mediated biological effect. In some embodiments a C5L2 inhibitor may be used to increase an IL-17 mediated biological effect. The term “IL-17 mediated biological effect” refers to any biological effect caused at least in part by IL-17. As used herein and in the art, “IL-17” refers to IL-17A. One of ordinary skill in the art will appreciate that IL-17F is similar to IL-17A and is generally expressed in a similar manner. Certain embodiments relating to IL-17 pertain to IL-17F.

In some embodiments a C5L2 activator may be used to inhibit, reduce or at least in part prevent an IFN-γ mediated biological effect. In some embodiments a C5L2 inhibitor may be used to increase an IFN-γ mediated biological effect. The term “IFN-γ mediated biological effect” refers to any biological effect caused at least in part by IFN-γ. IFN-γ has a variety of biological effects such as activating macrophages (e.g., to kill phagocytosed microbes), acting on B cells to promote or inhibit certain Ig isotype switching, and promoting differentiation of resting CD4+ cells to the Th1 subset.

In some embodiments a C5L2 activator may be used to inhibit, reduce or at least in part prevent an IL-6 mediated biological effect. In some embodiments a C5L2 inhibitor may be used to increase an IL-6 mediated biological effect. The term “IL-6 mediated biological effect” refers to any biological effect caused at least in part by IL-6. IL-6 is a pleiotropic cytokine involved in the physiology of a number of organ systems. Among other things, IL-6 plays an important role in inducing the development of Th17 cells from naïve T cells together with TGF-beta.

In some embodiments a C5L2 activator may be used to inhibit, reduce or at least in part prevent an IL-1β mediated biological effect. In some embodiments a C5L2 inhibitor may be used to increase an IL-1β mediated biological effect. The term “IL-1β mediated biological effect” refers to any biological effect caused at least in part by IL-1β. In some embodiments an IL-17 mediated biological effect, IFN-γ mediated biological effect, IL-1β mediated biological effect, or IL-6 mediated biological effect is a pathological effect, e.g., it causes damage to or dysfunction of one or more tissues or organs and/or is a symptom or sign of a disorder. In some embodiments a disorder is characterized by increased levels of at least one, two, three, or all of the foregoing cytokines.

In some embodiments, a C5L2 activator or C5L2 inhibitor may be used to modulate expression of a TGF-beta receptor chain, e.g., by a mammalian T cell.

It will be understood that a difference or alteration, e.g., an increase or decrease, in a parameter of interest (e.g., cytokine level, suppressive activity) may vary. For example, a difference or alteration (e.g., as compared to a reference value) may be an increase or decrease of the parameter of interest by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99%, or within any range between any two of the foregoing, in various embodiments. In some embodiments a difference or alteration may be an increase or decrease of the relevant parameter by at least about 1.5, 2, 3, 4, 5, 7.5, 10, 20, 30, 40, 50, 75, 100-fold or more, or within any range between any two of the foregoing, in various embodiments. In some embodiments an alteration is statistically significant. A reference value may be a value existing prior to or in the absence of a particular agent (e.g., a C5L2 modulator) or any suitable control value. In some embodiments an alteration in a first parameter may arise as a result of exposure to a C5L2 modulator, while a second parameter may remain substantially unchanged. For example, production of one or more cytokines may be altered while production of one or more other cytokines may remain substantially unchanged.

Methods described herein may in general be applied to individual cells or populations of cells, which may be in vitro or in vivo in various embodiments. In certain embodiments methods are applied to a population of cells in a culture vessel. In certain embodiments methods are applied to a population of cells in vivo. In certain embodiments a population of cells has at least a specified degree of purity with respect to cell type or cell subset, e.g., as assessed based on marker expression level (e.g., positive/high or negative/low) of one or more markers. For example, in some embodiments at least 50%, 60%, 70%, 80%, 90%, 95%, 96%, 97%, 98%, 99%, or more of the cells in a population of cells may exhibit a particular marker expression profile. In some embodiments a marker expression profile includes expression levels of 1, 2, 3, 4, 5, 6 markers, or more. One of ordinary skill in the art will be aware of methods that may be used to purify cells, e.g., from blood or tissue samples and/or to classify or sort cells based on cell surface markers. Methods may include positive selection, negative selection, or both. In certain embodiments antibodies to particular cell surface markers are used. Such antibodies may be used to deplete cells expressing the marker or to enrich for cells expressing the marker. In some embodiments an antibody is attached to a support such as a microparticle (sometimes termed a “bead”), which may be magnetic. In some embodiments an antibody has a fluorescent label conjugated thereto, which may be used to detect cells to which the antibody is attached. Such cells may then be separated from other cells using fluorescence activated cell sorting. Cells may be cultured in media appropriate for the particular cell type. In some embodiments, media may contain one or more cytokines or small molecules that promote survival and/or maintenance of the phenotype of a particular T cell subset. In some embodiments T cells may be activated in vitro by exposure to one or more cytokines (e.g., IL-2, IL-7 or IL-15) and/or one or more antibodies or ligands to cell surface molecules or complexes such as a T cell receptor (TCR), CD28, and/or CD46. For example, e.g., antibody to CD3 and antibody to CD28 and/or CD46 may be used to activate resting T cells. Such stimulation may mimic the activation that occurs in vivo when a T cell encounters an antigen to which its TCR binds, in the context of appropriate MHC and co-stimulatory molecules.

While the present disclosure focuses mainly on T cells, primarily CD4+ T cells, and monocytes, it is envisioned that other cells (immune system cells or other cells) may produce C5 and cleave C5 intracellularly to generate active fragments such as C5a. It is also envisioned that other cells may express membrane-bound carboxypeptidases that cleave C5a released by such cells to generate C5adesArg. It is also envisioned that other cells may express C5L2 and exhibit autocrine stimulation of C5L2 by C5a and/or C5adesArg arising from C5 produced by such cells. The particular effects of such autocrine stimulation may vary depending on cell type. In certain aspects, the present disclosure contemplates the use of C5L2 modulators such as those described or identified as described herein, for purposes of modulating the phenotype, differentiation, and/or functions of such cells.

Sequences of polypeptides of interest herein, e.g., complement factors, cytokines, receptors, enzymes, are well known in the art and available in public databases such as those available through Entrez at the National Center for Biotechnology Information (www.ncbi.nih.gov) or Universal Protein Resource (www.uniprot.org). Exemplary databases include, e.g., GenBank, RefSeq, Gene, Protein, Nucleotide, UniProtKB/SwissProt, UniProtKB/Trembl, and the like. In general, sequences, e.g., mRNA and polypeptide sequences, in the NCBI Reference Sequence database may be used as gene product sequences for a gene of interest. Such sequences may be used, e.g., to produce a polypeptide useful as an antigen or reagent for production, isolation, or characterization of an agent that binds to the gene product. It will be appreciated that multiple alleles of a gene may exist among individuals of the same species. For example, differences in one or more nucleotides (e.g., up to about 1%, 2%, 3-5% of the nucleotides) of the nucleic acids encoding a particular protein may exist among individuals of a given species. Due to the degeneracy of the genetic code, such variations often do not alter the encoded amino acid sequence, although DNA polymorphisms that lead to changes in the sequence of the encoded proteins can exist. Examples of polymorphic variants can be found in, e.g., the Single Nucleotide Polymorphism Database (dbSNP), available at the NCBI website at www.ncbi.nlm.nih.gov/projects/SNP/. (Sherry S T, et al. (2001). “dbSNP: the NCBI database of genetic variation”. Nucleic Acids Res. 29 (1): 308-311; Kitts A, and Sherry S, (2009). The single nucleotide polymorphism database (dbSNP) of nucleotide sequence variation in The NCBI Handbook [Internet]. McEntyre J, Ostell J, editors. Bethesda (Md.): National Center for Biotechnology Information (US); 2002 (www.ncbi.nlm.nih.gov/bookshelf/br.fcgi?book=handbook&part=ch5). Multiple isoforms of certain proteins may exist, e.g., as a result of alternative RNA splicing or editing. In general, where aspects of this disclosure pertain to a gene or gene product, embodiments pertaining to allelic variants or isoforms (where applicable) are encompassed unless indicated otherwise. Certain embodiments may be directed to particular sequence(s), e.g., particular allele(s) or isoform(s).

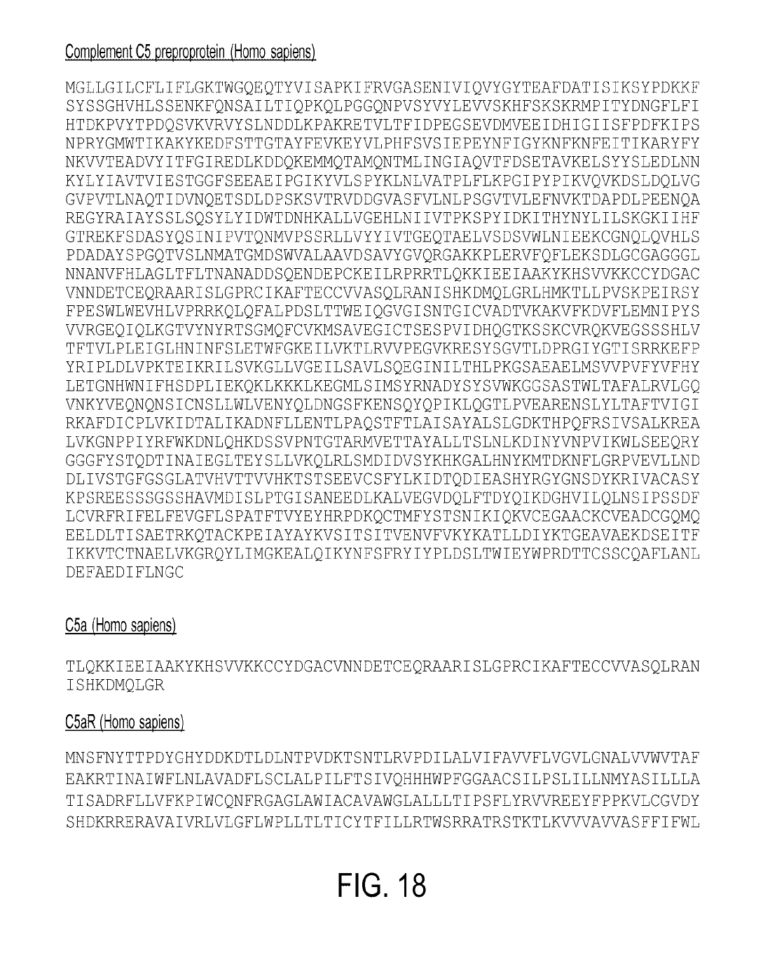

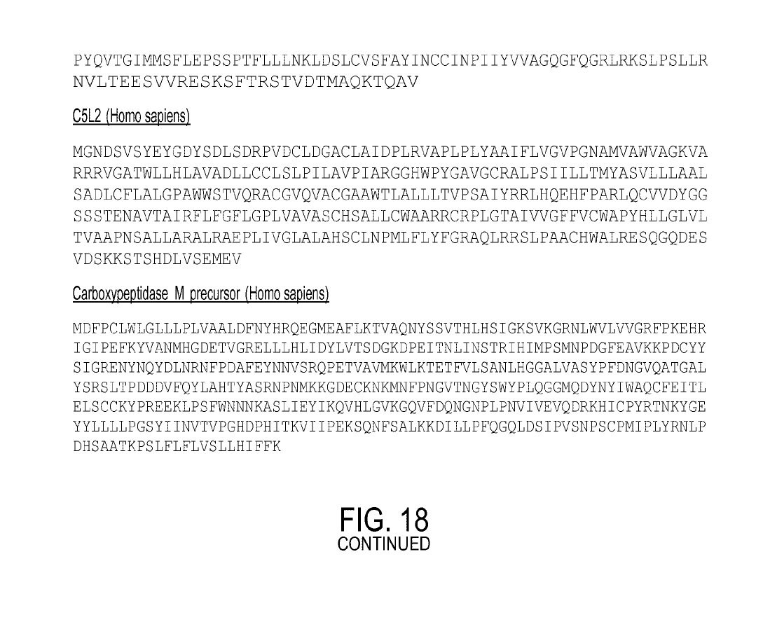

Table 1 provides Gene IDs and NCBI RefSeq accession numbers for certain human polypeptides of interest herein. Reference sequences for certain proteins are provided in FIG. 18. It will be appreciated that certain of the protein sequences are precursor sequences. The mature form of the protein may, for example, lack a secretion signal sequence present in the precursor. It will be appreciated that the sequences described under the respective accession numbers are exemplary and that naturally occurring variants, e.g., allelic variants, are encompassed in various embodiments. If an accession number version is updated either the present version or updated version may be used in various embodiments. Furthermore, it will be appreciated that for purposes of generating a useful binding agent (e.g., an antibody) for use, e.g., as a detection reagent or therapeutic agent, variant sequences or fragments (e.g., peptides), etc., may be used in certain embodiments.

TABLE 1
Gene ID and Accession Numbers for Certain Human Polypeptides

| | Alternate | | | Protein |
|---|---|---|---|---|
| Gene Official | names and | | mRNA accession | accession |
| Symbol/Name | comments | Gene ID | number | number |
| C5 | | 727 | NM_001735.2 | NP_001726.2 |
| C5AR1 | C5aR, C5R1, | 728 | NM_001736.3 | NP_001727.1 |
| | CD88 | | | |
| C5AR2 | C5L2, GPF77, | 27202 | NM_001271749.1 | NP_001258678.1 |
| | GPR77 | | | |
| CPM* | | 1368 | NM_001005502.2 | NP_001005502.1 |
| | | | NM_001874.4 | NP_001865.1 |
| | | | NM_198320.3 | NP_938079.1 |
| IL-17A | Il-17 | 3605 | NM_002190 | NP_002181 |
| IFN-γ | | 3458 | NM_000619.2 | NP_000610.2 |
| IL-1β | IL-1 | 3553 | NM_000576.2 | NP_000567.1 |
| IL-6 | BSF2, HGF, | 3569 | NM_000600.3 | NP_000591.1 |
| | HSF, IFNB2 | | | |

*The protein sequences under the three accession numbers for CPM are identical. There are three transcript variants.

### III. C5L2 Modulators

In certain embodiments a C5L2 activator is C5adesArg. In some embodiments C5adesArg may be purified from human serum. Purification may be performed by any of a variety of methods such as immunoadsorbent and molecular sieve chromatography (see, e.g., Manderino G L, et al., (1982) J Immunol Methods. 53(1):41-50. In certain embodiments C5adesArg is glycosylated at Asn64. In certain embodiments a C5L2 modulator is a variant of C5a or C5adesArg. In some embodiments the variant has an alteration at position 27, 67, 69, 70, 71, 72, and/or 73 as compared with C5a or C5adesArg. For example, D69 may be replaced by a positively charged amino acid such as arginine in a C5L2 antagonist. Certain C5a variants that are antagonists of C5aR or of both C5aR and C5L2 (dual C5aR/C5L2 antagonists) are described in Otto, M., et al., J. Biol. Chem. (2004) 279(1): 142-151, 2004, and/or in US Pat. Pub. No. 20060052294. An exemplary dual C5aR/C5L2 antagonist is known in the art as A8. Six positions are mutated in A8 as compared with C5a: C27A, H67F, D69R, M70S, Q71L, and G73R. In addition, Arg74 is deleted. In some embodiments, A8 or a variant lacking one or more of amino acids 71-73, e.g., A8Delta71-73, may be used. In some aspects, the present disclosure contemplates generating additional variants of C5a and identifying a variant that acts selectively as an antagonist for C5L2 versus C5a. In some aspects, it is contemplated to identify C5L2 agonists that have greater ability to activate C5L2 than does C5adesArg. In some aspects, it is contemplated to identify C5L2 antagonists that have greater ability to inhibit C5L2 than does A8. In some embodiments concatemers or multimers, e.g., dimers, comprising multiple C5L2 modulators, e.g., antagonists, optionally separated by linkers, may be used.

In certain embodiments a C5L2 modulator is an agent, e.g., an antibody or non-antibody polypeptide or aptamer, that binds to C5, C5a or C5adesArg. Agents that bind to intact C5 may block cleavage of C5 to C5a and C5b. In certain embodiments an agent binds to the alpha chain of C5. In certain embodiments the agent binds to a portion of the C5 alpha chain that, following activation of C5, is found in C5a. In some embodiments the agent specifically binds to C5a and/or C5adesArg and not to intact C5. Agents that bind to C5a and/or C5adesArg can act as C5L2 inhibitors by inhibiting binding of C5a and C5adesArg to C5L2. Exemplary monoclonal antibodies that bind to C5, C5a, and/or C5adesArg are described in EP Pub. No. 0245993, PCT/US1995/05688 (WO/1995/29697) or PCT/US2011/066437 (WO/2012/088247) and others are known in the art. In some embodiments it is contemplated to use a single domain or single chain antibody or polypeptide that binds to C5 or C5a. In some embodiments such an antibody may be rendered cell-permeable, e.g., as described further below. An exemplary single chain human monoclonal antibody that binds to C5 and inhibits its cleavage, referred to as TS-A 12/22, is described in Marzari, R., et al., (2002) Eur J Immunol. 32(10):2773-82. See also Fischetti, F., et al., (2007) Arthritis and Rheumatism, 56(4) 1187-1197.

In certain embodiments a C5L2 modulator is an agent that binds to C5L2, e.g., an anti-05L2 antibody. Exemplary blocking antibodies that bind to C5L2 are described in Lee, et al. and Bamberg, et al., (both cited above). Mouse monoclonal antibodies to human C5L2 known in the art include clone 1D9-M12 (Biolegend, San Diego, Calif.) and ab167121 (Abcam, Cambridge, UK, and Cambridge, Mass.). It is contemplated that antibodies that activate C5L2 may be identified.

In some embodiments a C5L2 activator comprises a carboxypeptidase capable of cleaving C5a to form C5adesArg. In some embodiments a C5L2 inhibitor inhibits expression or activity of an endogenous carboxypeptidase that is capable of cleaving C5a to form C5adesArg. Carboxypeptidases are enzymes that catalyze the hydrolysis of the C-terminal peptide bond in peptides and proteins. They are involved in a variety of biological processes such as protein digestion, modulation of hormone activities, hemostasis, and inflammation. CPs are broadly classified based on structure, substrate specificity, and biological function into a digestive/pancreatic subfamily (CPA1-CPA6, CPB1, and CPB2 (also known as CPU and CPR) and a regulator subfamily (CPE/H, CPN, CPD, CPZ, CPX1, CPX2, and CPM). CPM, CPE/H, CPN, CPD, CPZ, CPB1, and CPB2 cleave C-terminal arginine or lysine, with different specificities. In certain embodiments the carboxypeptidase may be any of a variety of carboxypeptidases capable of cleaving N-terminal to an arginine residue located at the C-terminus of a protein (e.g., Arg74 of C5a). For example, the carboxypeptidase may be carboxypeptidase M (CPM) or carboxypeptidase N (CPN). CPM is unique among CPs known thus far in that it inserts via a glycosylphosphatidylinositol (GPI) anchor into the plasma membranes of various cell types. A soluble form of CPM has also been demonstrated in certain body fluids. As described herein, Applicants discovered that CMP is expressed in resting and activated T cells and monocytes and is responsible for generating C5adesArg from C5a produced in these cells. CPN is the major CP present in the blood that cleaves the C-terminal arginine from anaphylatoxins. CPN is produced in the liver and secreted into the bloodstream. CPN is composed of an ˜83 kDa non-catalytic regulatory subunit and a 55 kDa catalytic subunit that is cleaved to an ˜48 kDa active form.

In some embodiments of interest, a C5L2 modulator comprises a CPM or CPM modulator. In some embodiments a C5L2 activator comprises at least a catalytically active portion of an extracellular domain of CPM or a variant thereof. In some embodiments the catalytically active portion of CPM or variant thereof is soluble, e.g., it lacks a GPI anchor signal sequence, such that the protein does not become attached to the cell membrane via a GPI anchor. For example, in some embodiments the catalytically active portion of CPM lacks at least about the C-terminal 10-15 amino acids, or about the C-terminal 15-20 amino acids, and/or has an alteration of See⁴⁰⁶ (the putative site of GPI anchor attachment) to an amino acid such as proline that is not capable of serving efficiently as a GPI anchor attachment site. In some embodiments a CPM or fragment or variant thereof, e.g., a soluble, catalytically active portion of CPM, may be expressed in a eukaryotic expression system such as a baculovirus-infected insect cell expression system. In certain embodiments a GPI-anchored CPM may be cleaved by a phosphatidylinositol-specific phospholipase C such as bacterial PI-PLC to release a soluble, catalytically active protein. In certain embodiments expression and/or purification of a soluble CPM may be performed as described in Tan, F., et al., (2003) Biochem. J., 370, 567-578. For example, CPM may be purified using ion exchange chromatography and/or based on affinity for arginine (e.g., using arginine-sepharose). Also described by Tan (2003) are various alterations to the sequence of CPM that either do or do not cause the resulting protein to lose activity or be secreted rather than attached to the cell membrane. In some embodiments a C5L2 inhibitor comprises a CP inhibitor, e.g., a CPM inhibitor. In some embodiments, a CPM inhibitor may mimic an endogenous substrate of CPM in binding to CPM but may be noncleavable. Activity of a CP capable of cleaving N-terminal to arginine may be measured using an artificial substrate such as a dansyl-Ala-Arg substrate, e.g., as described in Tan, F., et al. (1995) Methods Enzymol. 248, 663-675. Antibodies to CPM are known in the art. For example, NCL-CPMm is a mouse monoclonal antibody (IgG1, kappa) to human CPM (clone 1C2, mouse myeloma (p3-NS1-Ag4-1) available from Leica Biosystems (Newcastle Upon Tyne, UK) (formerly offered by Novocastra Laboratories) (Tan, et al., 2003, cited above).

In some embodiments a carboxypeptidase inhibitor is DL-2-mercaptomethyl-3-guanidinoethylthiopropanoic acid (also known as Mergepta) or a structurally similar compound. In some embodiments a CP inhibitor is potato-derived carboxypeptidase inhibitor.

In some embodiments a C5L2 modulator comprises an RNAi agent, such as a short interfering RNA (siRNA) or artificial microRNA, that inhibits expression of C5L2 or CPM by RNA interference. In some embodiments a C5L2 modulator that binds to C5L2 or CPM comprises an engineered polypeptide distinct from antibodies, such as an adnectin, affibody, anticalin, or darpin.

In certain embodiments a C5L2 modulator is selective for C5L2 as compared with C5aR. For example in certain embodiments the binding affinity of a C5L2 modulator for C5L2 is greater than for C5a. In certain embodiments an RNAi agent comprises a sequence that has a higher degree of complementarity to C5L2 mRNA than to C5aR mRNA.

In certain embodiments it is contemplated that C2a, C4a, and/or C4adesArg, or variants of C2a, C4a, and/or C4adesArg may be used as C5L2 modulators. In certain embodiments C2a, C4a, and/or C4adesArg, or variants of C2a, C4a, and/or C4adesArg may be used to activate C5L2. In certain embodiments C2a, C4a, and/or C4adesArg, or variants of C2a, C4a, and/or C4adesArg may be used to inhibit C5L2. In certain embodiments it is contemplated that C3a and/or C3adesArg or variants of C3a and/or C3adesArg may be used as C5L2 modulators. In certain embodiments C3a and/or C3adesArg, or variants of C3a and/or C3adesArg may be used to activate C5L2. In certain embodiments C3a and/or C3adesArg, or variants of C3a and/or C3adesArg may be used to inhibit C5L2.

A variety of methods may be used to measure the ability of an agent to bind to a cell-bound target molecule such as C5L2, C5aR, or CPM. In some embodiments an agent is labeled with a detectable moiety, incubated with cells that express the target molecule, and washed to remove unbound agent. A detectable moiety may be, e.g., a radiolabel, fluorescent small molecule or protein, epitope tag, or enzyme. Agent that remains physically associated with the cell is detected. In some embodiments cells that do not express the target molecule are used as a control. In some embodiments a cell line that does not express the target molecule is transfected with a nucleic acid construct encoding the target. The transfected cell line is used as a target for measuring binding of a test agent, and the parental cell line is used as a control. In some embodiments functional assays may be used.

In certain embodiments, a C5L2 modulator may be physically associated with, e.g., conjugated to, a polypeptide or non-polypeptide component of use to stabilize the compound, reduce its immunogenicity, increase its lifetime in the body, increase its solubility, and/or increase its resistance to degradation. In some aspects a moiety such as a polyethylene glycol (PEG) chain or other polymer(s) that, e.g., stabilize the compound, increase its lifetime in the body, increase its solubility, decrease its immunogenicity, and/or increase its resistance to degradation may be referred to herein as a “clearance reducing moiety” (CRM). In some embodiments a C5L2 modulator may be physically associated with, e.g., conjugated to, a targeting moiety, a cell uptake moiety, a cell-reactive moiety, or a cell membrane binding moiety. In certain embodiments the physical association may be via a covalent or non-covalent bond.

In certain embodiments, a polymer such as polyethylene glycol (PEG), albumin, or an albumin-binding peptide, may be used. Methods for pegylation are well known in the art (Veronese, F. M. & Harris, Adv. Drug Deliv. Rev. 54, 453-456, 2002; Davis, F. F., Adv. Drug Deliv. Rev. 54, 457-458, 2002); Hinds, K. D. & Kim, S. W. Adv. Drug Deliv. Rev. 54, 505-530 (2002; Roberts, M. J., Bentley, M. D. & Harris, J. M. Adv. Drug Deliv. Rev. 54, 459-476; 2002); Wang, Y. S. et al. Adv. Drug Deliv. Rev. 54, 547-570, 2002). A wide variety of polymers such as PEGs and modified PEGs, including derivatized PEGs to which polypeptides can conveniently be attached are known in the art. In another embodiment a C5L2 modulator is fused to the Fc domain of an immunoglobulin or a portion thereof. In some other embodiments a C5L2 modulator is conjugated to an albumin moiety or to an albumin binding peptide. In some embodiments, a polyethylene glycol (PEG) or other clearance reducing moiety has a molecular weight of at least about 5, 10, 20, 30, 40, 50, 60, 70, 80, 90, 100, 120, or 150 kilodaltons (kD).

In some embodiments, a C5L2 modulator disclosed herein may be extended or modified by addition of a linker comprising one or more amino acids, e.g., one or more amino acids comprising a primary or secondary amine, e.g., in a side chain thereof. For example, a Lys residue, or a sequence comprising a Lys residue, is added at the N-terminus and/or C-terminus of a polypeptide. In some embodiments, the Lys residue is separated from an active domain of the polypeptide by a rigid or flexible spacer. A linker or spacer may, for example, comprise a substituted or unsubstituted, saturated or unsaturated alkyl chain, oligo(ethylene glycol) chain, and/or other moieties. The length of the chain may be, e.g., between 2 and 20 carbon atoms. In some embodiments the spacer is or comprises a peptide. The peptide spacer may be, e.g., between 1 and 20 amino acids in length, e.g., between 4 and 20 amino acids in length. Suitable spacers can comprise or consist of multiple Gly residues, Ser residues, or both, for example. Optionally, the amino acid having a side chain comprising a primary or secondary amine and/or at least one amino acid in a spacer is a D-amino acid. A PEG moiety or similar molecule or polymeric scaffold may be linked to the primary or secondary amine, optionally via a linker. In some embodiments, a bifunctional linker is used. Abifunctional linker may comprise two reactive functional groups, which may be the same or different in various embodiments. In various embodiments, one or more linkers, spacers, and/or techniques of conjugation described in Hermanson, cited above, is used. Any of a variety of polymeric backbones or scaffolds could be used. For example, the polymeric backbone or scaffold may be a polyamide, polysaccharide, polyanhydride, polyacrylamide, polymethacrylate, polypeptide, polyethylene oxide, or dendrimer. Suitable methods and polymeric backbones are described, e.g., in WO98/46270 (PCT/US98/07171) or WO98/47002 (PCT/US98/06963). In one embodiment, the polymeric backbone or scaffold comprises one or more reactive functional groups, such as carboxylic acids, anhydride, or succinimide groups. The polymeric backbone or scaffold is reacted with the C5L2 modulator. In one embodiment, the C5L2 modulator comprises any of a number of different reactive functional groups, such as carboxylic acids, anhydride, or succinimide groups, which are reacted with appropriate groups on the polymeric backbone or scaffold.

In some embodiments a targeting moiety targets the agent to a cell, tissue, or location in the body at which C5L2 modulation is desired. In some embodiments a targeting moiety comprises, e.g., an antibody, polypeptide, peptide, nucleic acid (e.g., an aptamer), carbohydrate, small molecule (e.g., a receptor ligand), or supramolecular complex, that specifically binds to a target molecule. In some embodiments, the affinity (as measured by the equilibrium dissociation constant, Kd) of targeting moiety for the target molecule (as measured by the equilibrium dissociation constant, Kd) is 10⁻³ M or less, e.g., 10⁻⁴ M or less, e.g., 10⁻⁵ M or less, e.g., 10⁻⁶M or less, 10⁻⁷M or less, 10⁻⁸M or less, or 10⁻⁹ M or less under the conditions tested, e.g., under physiological conditions. In some embodiments, a target molecule is characteristic of a particular diseased or physiological state or characteristic of one or more cell type(s) or tissue type(s). A target molecule is often a molecule at least partly present at the cell surface (e.g., a transmembrane or otherwise membrane-attached protein) so that at least a portion of the molecule is accessible to binding by an extracellular binding agent such as an antibody. In certain embodiments the target molecule is exposed at the surface of a target cell which, in some embodiments, is a T cell, a monocyte, a cancer cell, a pathogen, or a pathogen-infected cell. A target molecule expressed by a cell may, but need not be, cell type specific. For example, a cell type specific target molecule is often a protein, peptide, mRNA, lipid, or carbohydrate that is present at a higher level on or in a particular cell type or cell type(s) than on or in many other cell types. In some instances a cell type specific target molecule is present at detectable levels only on or in a particular cell type of interest. However, it will be appreciated that a useful cell type specific target molecule need not be absolutely specific for the cell type of interest in order to be considered cell type specific. One of ordinary skill in the art will be aware of cell surface markers that may be used as targets. In some embodiments a CD molecule is used. In some embodiments the target is a protein that is not required or important for cell survival and/or for a desired functional activity of the cell. In some embodiments a target molecule may be present at a site of tissue inflammation or tissue damage, may be a pathogen-derived molecule, or may be a tumor antigen.

In some embodiments, it is contemplated to modulate intracellular generation of C5a, modulate secretion of C5a, and/or modulate intracellular activity of C5aR and/or C5L2. In some embodiments, cells, e.g., T cells or monocytes, are contacted with a cell-permeable C5L2 modulator, C5aR modulator, or C5a binding agent. “Cell-permeable” in this context refers to a substance that can cross cell membranes of living eukaryotic, e.g., mammalian, e.g., human cells, in a sufficient amount to be detectable therein and, in some embodiments, exert a biological effect therein. In some embodiments, a C5L2 modulator, C5aR modulator, or C5a binding agent that may otherwise be poorly cell permeable (have low or essentially no ability to cross the cell membrane) may be physically associated with a cell uptake moiety. “Cell uptake moiety” refers to an entity that can be internalized by a living cell and is capable of delivering or enhancing delivery of a cargo to the interior of the cell. A cargo may be, e.g., a peptide, protein, nucleic acid, small molecule, or nanoparticle or other entity of similar dimensions. The term “internalized by a cell” refers to gaining access to the interior (inside) of the cell. The “interior of a cell” refers to locations within the boundary of the plasma membrane. For purposes hereof, membrane-bound intracellular vesicles and their contents are considered to be inside the cell. Internalization may occur via endocytotic processes and/or non-endocytotic processes (e.g., pinocytosis, direct penetration, and transporter-mediated uptake) in various embodiments. Entities that are contained in vesicles inside the cell, e.g., following endocytosis or pinocytosis, may be released from such vesicles and enter the cytoplasm by various routes. For example, such entities may undergo retrograde transport from vesicles to the interior of the endoplasmic reticulum (ER) and translocate from there into the cytoplasm or may directly translocate across vesicular membranes.

In some embodiments a cell uptake moiety is capable of entering at least some immune system cells, e.g., lymphocytes (e.g., T cells), granulocytes (e.g., neutrophils), mast cells, monocytes, and/or macrophages. In some embodiments a cell uptake moiety comprises a cell penetrating peptide (CPP), sometimes referred to as a “protein transduction domain”. Such peptides can be internalized by a cell and delivering or enhancing delivery of a cargo to the interior of the cell. Naturally occurring CPPs occur in a number of different naturally occurring proteins including various viral proteins, animal proteins (e.g., insect, mammalian), and plant proteins. CPPs have been identified in certain secreted proteins, transcription factors, venoms, and toxins, among others. They are typically linear sequences ranging from about 6 to about 30 amino acids in length that are able to mediate transport of the protein in which they occur into cells. In some embodiments a CPP comprises or consists of such a naturally occurring amino acid sequence. In some embodiments a CPP comprises or consists of a non-naturally occurring amino acid sequence, i.e., an amino acid sequence not known to occur in nature either alone or as part of a longer polypeptide. A non-naturally occurring CPP may comprise a variant of a naturally occurring CPP, a chimeric sequence comprising portions of two or more naturally occurring CPPs, or a sequence designed to have one or more properties of a naturally occurring CPP wherein such property correlates with and/or is believed to be at least in part responsible for the ability of the naturally occurring CPP to be internalized by a cell and/or to enter a particular subcellular compartment (e.g., the cytoplasm) or organelle. In some embodiments a CPP is derived from a different CPP or from a polypeptide (e.g., a naturally occurring polypeptide able to enter cells). For purposes of this disclosure, a CPP is considered to be “derived from” a particular polypeptide if the CPP (i) comprises or consists of a fragment of the polypeptide, wherein the fragment is at least 6 amino acids long, e.g., 6, 7, 8, 9, 10, 11, 12, 13, 14, 15, 16, 17, 18, 19, 20, 21, 22, 23, 24, 25, 26, 27, 28, 29, or 30 amino acids long; (ii) comprises or consists of a peptide that is at least 70% identical to a fragment of the polypeptide that is at least 10 amino acids long; (iii) comprises or consists of a peptide whose sequence can be generated by making no more than 3 alterations (which may be substitution(s), deletion(s), or addition(s), in any combination) to the sequence of a fragment of the protein that is at least 10 amino acids long; (iv) comprises or consists of a peptide that is a cyclized, circularly permuted, inverso, retro, or retro-inverso version of a peptide as described in any of (i), (ii), or (iii). In certain embodiments any of the peptides of (i), (ii), (iii), or (iv) may have one or more modifications to one or more side chains, backbone, and/or to an N- or C-terminus. As will be appreciated, an inverso version of a peptide is the D-enantiomer of the peptide and has the same sequence as the peptide but is composed of D-amino acids and has a mirror conformation; a retro version of a peptide consists of the same sequence of L amino acids but in reverse order; a retro-inverso version of a peptide consists of D-amino acids in the reverse order and is the D-retro-enantiomer of the peptide. In some embodiments a cell penetrating moiety may be related to a CPP in that the CPM is designed, generated, derived, etc., from or based on the CPP, e.g., using a design principle or experimental approach intended to preserve, mimic, enhance, or select for ability to be internalized by a cell and/or to enter a particular subcellular compartment (e.g., the cytoplasm or an organelle).

In general, many CPPs may be broadly classified as cationic, hydrophobic, or amphipathic peptides. The term “cationic peptide” refers to a peptide that has a positive average net charge when in water at a physiological pH, e.g., a pH of 7.0-7.4. In some embodiments a CPP comprises or consists of a cationic peptide at least 6 amino acids long, e.g., 6-12, 12-20, or 20-30 amino acids long. In some embodiments at least 30%, 40%, 50%, 60%, 70%, 80%, 90%, or 100% of the amino acids in a cationic peptide are basic amino acids. In some embodiments a basic amino acid comprises a side chain that has a pKₐ of at least 8.0, at least 9.0, or at least 10.0 in water. In general, a basic amino acid may be a standard amino acid or a non-standard amino acid. In some embodiments a basic amino acid comprises a side chain comprising a primary or secondary amine or a guanidinium group. In some embodiments at least 30%, 40%, 50%, 60%, 70%, 80%, 90%, or 100% of the amino acids in a cationic peptide are independently selected from arginine, ornithine, lysine, and basic analogs of any of these. In some embodiments at least 30%, 40%, 50%, 60%, 70%, 80%, 90%, or 100% of the amino acids in a cationic peptide are independently selected from arginine, lysine, and basic analogs of either of these. In some embodiments at least 30%, 40%, 50%, 60%, 70%, 80%, 90%, or 100% of the amino acids in a cationic peptide are arginine or lysine. In some embodiments at least 30%, 40%, 50%, 60%, 70%, 80%, 90%, or 100% of the amino acids in a cationic peptide are arginine. A basic analog of a basic amino acid may comprise substituent(s) at any one or more positions, so long as the resulting compound retains a net positive charge. In some embodiments a substituent is a lower alkyl or lower alkanoyl group.

In some embodiments a CPP comprises a hydrophobic peptide at least 6 amino acids long, e.g., 6-12, 12-20, or 20-30 amino acids long. In general, a hydrophobic peptide is composed predominantly of hydrophobic and neutral amino acids. In some embodiments a hydrophobic peptide comprises at least 50%, 60%, 70%, 80%, 90%, or more hydrophobic and neutral amino acids. A neutral amino acid may be selected from alanine, isoleucine, leucine, valine, phenylalanine, tryptophan, tyrosine, cysteine, methionine, threonine, glycine, serine, glutamine, and neutral analogs thereof. Unless otherwise indicated or evident from the context or use, “neutral” refers to neutral (uncharged) within a physiological pH range, e.g., between 7.0 and 7.4. A neutral analog of an amino acid may comprise a neutral substituent as compared with the amino acid of which it is an analog. A hydrophobic amino acid may be selected from alanine, isoleucine, leucine, valine, phenylalanine, tryptophan, tyrosine, cysteine, methionine, and hydrophobic analogs of any of the foregoing nine amino acids, wherein the hydrophobicity of a hydrophobic analog falls within the range of hydrophobicities of the foregoing nine amino acids or exceeds the upper limit of the range when measured using the same or substantially the same method and conditions as used to determine the range. In some embodiments a hydrophobic analog is an amino acid that has increased hydrophobic character as compared with the amino acid of which it is an analog. Increased hydrophobic character may, for example, result from presence of one or more additional hydrophobic groups or atoms in a side chain. In general, a hydrophobic group may be unsubstituted or substituted, provided that the substituent(s), if present, are sufficiently hydrophobic so as to not reduce the overall hydrophobicity of the amino acid below the lower limit of the afore-mentioned range. In some embodiments a hydrophobic group comprises or consists of an alkyl group, alkoxy group, or monocyclic or bicyclic aromatic ring. In some embodiments an alkyl group is a lower alkyl, e.g., methyl or ethyl. In some embodiments an alkoxy group is a lower alkoxy, e.g., methoxy or ethoxy. In some embodiments increased hydrophobic character results from presence of one or more additional —CH₂— groups in an alkyl chain. In some embodiments a hydrophobic group or substituent comprises a halogen. In some embodiments a hydrophobic substituent is present at one or more atoms that form part of the peptide backbone. In some embodiments a hydrophobic peptide comprises at least a portion of a signal sequence. A number of examples of hydrophobic peptides are provided herein. Peptide or amino acid hydrophobicity may be measured using a variety of methods. In some embodiments reverse phase HPLC may be used. The term “amphipathic peptide” refers to a peptide that possesses at least one hydrophilic region and at least one hydrophobic region. In some embodiments the hydrophilic and hydrophobic regions are present in distinct portions of the peptide sequence (primary structure). In some embodiments a CPP comprises an amphipathic peptide at least 6 amino acids long e.g., 6-9, 9-12, 12-20, or 20-30 amino acids long. In some embodiments the sequence of an amphipathic peptide comprises at least one sequence at least 4 amino acids long composed predominantly of hydrophilic amino acids and at least one sequence at least one sequence at least 4 amino acids long composed predominantly of hydrophobic amino acids. In some embodiments the amphiphilic character of an amphipathic peptide results at least in part from its secondary structure. For example, in some embodiments an amphipathic peptide comprises a helix, e.g., an alpha helix, having predominantly hydrophilic amino acid residues aligned along one side of the helix and predominantly hydrophobic amino acid residues aligned along the opposite side. The term “predominantly” is used to mean at least 75%, 80%, 85%, 90%, 95%, or 100%. Presence of a helix, e.g., an alpha helix, may be determined experimentally (e.g., spectrophotometrically, e.g., by circular dichroism spectroscopy in the far-ultraviolet (far-UV) spectral region (170-250 nm) or infrared spectroscopy), or may alternately or additionally be predicted using various computer programs or algorithms, such as the Chou-Fasman algorithm (Chou, P. Y., et al. (1974) Biochemistry, 13: 222-45) or a modified version thereof (see, e.g., Chen H, Gu F, Huang Z (2006). “Improved Chou-Fasman method for protein secondary structure prediction”. BMC Bioinformatics 7 (Suppl 4): S14), or other suitable algorithms or programs known in the art.

In some embodiments a CPP is both cationic and amphipathic.

A CPP may be linear or cyclic. A cyclic CPP may be cyclized via a bond between the N- and C-termini, a bond between a terminus and a side chain, a bond between two side chains, or a bond between the backbone and a side chain or via a linker.

Table 2 lists a variety of CPPs that may be used in various embodiments. The numbers in parentheses following a protein name herein indicate the first and last amino acids in a fragment of the protein. For example, Tat (49-56) refers to a peptide whose sequence consists of amino acids 49-56 of Tat. In some embodiments a CPP that is derived from or related to a CPP listed in Table 2 may be used. Table 2 also provides in some instances, whether a peptide is composed of L or D amino acids, the name of a peptide or protein in which a CPP is found or from which a CPP is derived. Lower case letters represents D-amino acids.

TABLE 2
Various CPPs

| | |
|---|---|
| GRKKRRQRRRPPQ | L Tat (48-60) HIV-1 |
| GISYGRKKRRQRRRPPQ | L Tat (43-60) HIV-1 |
| FITKALGISYGRKKRRQRRRPPQ | L Tat (37-60) HIV-1 |
| GRKKRRQRRR | L Tat (48-57) HIV-1 |
| RKKRRQRRR | L Tat (49-57) HIV-1 |
| RKKRRQRR | L Tat (49-56) HIV-1 |
| rkkrrqrrr | D D-Tat (49-57) HIV-1 |
| RRRQRRKKR | L Retro-Tat (57-49) HIV-1 |
| rrrqrrkkr | D D-Tat (57-49) HIV-1 |
| RKKRRARRR | L Ala54 substitution mutant of Tat (49-57) HIV-1 |
| GRKKRRQRRRC | L Pro deletion mutant of Tat (48-60) HIV-1 |
| TRQARRNRRRRWRERQR | L Rev (34-50) HIV-1 |
| GWTLNSAGYLLGPHAVGNHRSFSDKNGLTS | L Galanin |
| INLKALAALAKKIL | L MP Wasp venom peptide Mastoparan |
| RQIKIWFQNRRMKWKK | L Antennapedia home odomain of drosophila |
| RQIKIWFQNRRMKWKK | L pAntpHD (43-58) Antennapedia |
| KKWKMRRNQFWIKIQR | L pAntpHD (58-43) Antennapedia |
| rqikiwfqnrrmkwkk D | D form of pAntpHD (43-58) Antennapedia |
| RQIKIWFPNRRMKWKK | L pAntpHD (Pro50) Antennapedia |
| RQPKIWFPNRRKPWKK | L pAntpHD (3Pro) Antennapedia |
| RQIKIWFQNRRMKWKK | L pAntp (43-58) Antennapedia |
| RQIKIWFQNRRMKWK | L pAntp (43-57) Antennapedia |
| RQIKIWFQNRRMKW | L pAntp (43-56) Antennapedia |
| IKIWFQNRRMKWKK | L pAntp (45-58) Antennapedia |
| RQIKIWFPNRRMKWKK | L Penetratin (pAntp) (43-58) Antennapedia |
| RAAARQARAG | L PTD4 |
| YARAAARQARAG | L PTD4 |
| KMDCRWRWKCCKK | L Crot (27-39) Retal snake venom (Crotamine) |
| RKKRRRESRKKRRRES | L DPV3 Human Superoxide dismutase |
| GRPRESGKKRKRKRLKP | L DPV6 Human platelet-derived growth factor |
| GKRKKKGKLGKKRDP | L DPV7 Human Epidermal-like growth factor |
| SRRARRSPRESGKKRKRKR | L DPV10/6 |
| VPMLK | L Bip1 Bax-binding domain of human Ku70 |
| KLPVM | L Bip9 Bax-binding domain of human Ku70 |
| TKRRITPKDVIDVRSVTTEINT | L Inv3 Mycobacterium cell entry protein (Mce1A) |
| AEKVDPVKLNLTLSAAAEALTGLGDK | L Inv5 Mycobacterium cell entry protein (Mce1A |
| TKRRITPKDVIDVRSVTTKINT | L Inv3.5 Mycobacterium cell entry protein (Mce1A) |
| KLIKGRTPIKFGKADCDRPPKHSQNGMGK | L Res1 L3 loop of restrictocin |
| KRIPNKKPGKKTTTKPTKKPTIKTTKKDLKPQTTKPK | L RSV-A1 Human respiratory syncytial |
| | virus, type A |
| DRRRRGSRPSGAERRRRRAAAA | L RSG 1.2 Arg-rich peptide |
| GTKMIFVGIKKKEERADLIAYLKKA | L Cyt C 71-101 Human Cytochrome C |
| RRRRNRTRRNRRRVRGC | L FHV coat (35-49) RNA Binding Peptides |
| MIIYRDLISKK | L TCTP-CPP 1 Human translationally controlled |
| | tumor protein |
| MIIYRDKKSH | L TCTP-CPP 2 Human translationally controlled |
| | tumor protein |
| MIIFRDLISH | L TCTP-CPP 3 Human translationally controlled |
| | tumor protein |
| MIIYRDLISH | L TCTP Human translationally controlled tumor |
| | protein |
| RRRRRRRR | L R8 |
| RRRRRRRRR | L R9 |
| rrrrr | D D-R6 |
| rrrrrrr | D D-R7 |
| rrrrrrrr | D D-R8 |
| rrrrrrrrr | D D-R9 |
| GWTLNSAGYLLGKINLKALAALAKKIL | L Transportan (TP) |
| ALWKTLLKKVLKAPKKKRKV | L S4(13)-PV Dermaseptin S4 peptide + SV40 NLS |
| EEEAAGRKRKKRT | L Glu-Oct-6 Transcription factor Oct-6 based |
| | chimeric peptide |
| KETWWETWWTEWSQPKKKRKV | L Pep-1 |
| GLRRLRQRRRLRRERVRA | L human neurturin |
| AAVALLPAVLLALLAP | L |
| KWKLFKKIGAVLKVL | L |
| KKLFKKILKYL | L |

In some embodiments a cell-reactive moiety comprises a reactive functional group that reacts with a functional group exposed at a cell surface to form a covalent bond. A cell membrane binding moiety may be any moiety that has affinity for eukaryotic, e.g., mammalian, cell membranes. Such affinity may result from one or multiple noncovalent interactions. In some embodiments a cell membrane binding moiety has affinity for a lipid, glycolipid, or phospholipid component of a cell membrane. In some embodiments a cell membrane binding moiety comprises at least one lipophilic binding element, optionally comprising one or more comprising aliphatic acyl groups. In some embodiments a cell membrane binding moiety may comprise a hydrophilic peptide, optionally having a lipophilic binding element linked to the hydrophilic peptide. Examples of certain cell membrane binding moieties are described in US Pat. Pub. No. 20040266684. In some embodiments the lipophilic binding element comprises 8 to 18 methylene units, or 10 to 14 methylene units. In some embodiments the lipophilic binding element comprises myristoyl. In some embodiments the hydrophilic peptide may comprise basic amino acids, e.g., at least 50% basic amino acids such as lysine. Examples of amino acid sequences comprising basic amino acids include: (i) DGPSPSKSSG (ii) GSSKSPSKKKKKKPGD (iii) SPSNETPKKKKKRFSFKKSG (iv) DGP SPSKSSK (v) SKDGKKKKKKSKTK.

In some aspects, a C5L2 modulator comprises a compound of formula M-L-A wherein A comprises a clearance reducing moiety, a targeting moiety, a cell uptake moiety, a cell-reactive moiety, or a cell membrane binding moiety; L is an optionally present linking portion; and M comprises a C5L2 modulator. The C5L2 modulator can comprise any of the C5L2 modulators described above, in various embodiments. In certain embodiments an agent may comprise one, two, or more selected from a clearance reducing moiety, a targeting moiety, a cell uptake moiety, a cell-reactive moiety, or a cell membrane binding moiety. Bonds between A and L or between L and M or between A and M may be covalent or noncovalent in various embodiments. Bonds may be between any atoms of the respective moieties. For example, L-A may be attached to the N-terminus, the C-terminus, or a side chain of an amino acid. In certain embodiments the same or different L-A units may be present at both ends of M. It will be appreciated that when certain agents are present in a compound of formula M-L-A, functional group of the agent(s) may have reacted with other functional groups to form a covalent bond. For example, an amino acid with a side chain containing a primary amine (NH₂) group (which can be represented as R¹—(NH₂)), can have a formula R¹—NH-L-A in which a new covalent bond to L (e.g., N—C) has been formed and a hydrogen lost. In some embodiments, L comprises an unsaturated moiety such as —CH═CH— or —CH₂—CH═CH—; a moiety comprising a non-aromatic cyclic ring system (e.g., a cyclohexyl moiety); an aromatic moiety (e.g., an aromatic cyclic ring system such as a phenyl moiety); an ether moiety (—C—O—C—); an amide moiety (—C(═O)—N—); an ester moiety (—CO—O—); a carbonyl moiety (—C(═O)—); an imine moiety (—C═N—); a thioether moiety (—C—S—C—); an amino acid residue; a (CH₂CH₂O)ₙ moiety, and/or any moiety that can be formed by the reaction of two compatible reactive functional groups. In certain embodiments, one or more moieties of a linking portion is/are substituted by independent replacement of one or more of the hydrogen (or other) atoms thereon with one or more moieties including, but not limited to aliphatic; aromatic, aryl; alkyl, aralkyl, alkanoyl, aroyl, alkoxy; thio; F; Cl; Br; I; —NO2; —CN; —CF3; —CH2CF3; —CHCl2; —CH2OH; —CH2CH2OH; —CH2NH2; —CH2SO2CH3; or -GRG1 wherein G is —O—, —S—, —NRG2-, —C(═O)—, —S(═O)—, —SO2-, —C(═O)O—, —C(═O)NRG2-, —OC(═O)—, —NRG2C(═O)—, —OC(═O)O—, —OC(═O)NRG2-, —NRG2C(═O)O—, —NRG2C(═O)NRG2-, —C(═S)—, —C(═S)S—, —SC(═S)—, —SC(═S)S—, —C(═NRG2)-, —C(═NRG2)O—, —C(═NRG2)NRG3-, —OC(═NRG2)-, —NRG2C(═NRG3)-, —NRG2SO2-, —NRG2SO2NRG3-, or —SO2NRG2-, wherein each occurrence of RG1, RG2 and RG3 independently includes, but is not limited to, hydrogen, halogen, or an optionally substituted aliphatic, aromatic, or aryl moiety. It will be appreciated that cyclic ring systems when present as substituents may optionally be attached via a linear moiety. Combinations of substituents and variables envisioned by this invention are preferably those that result in the formation of stable compounds useful in any one or more of the methods described herein, e.g., useful for the treatment of one or more disorders and/or for contacting a cell, tissue, or organ, as described herein, and/or useful as intermediates in the manufacture of one or more such compounds.

L can comprise one or more of any of the moieties described in the preceding paragraph, in various embodiments. In some embodiments, L comprises two or more different moieties linked to one another to form a structure typically having a length of between 1 to about 60 atoms, between 1 to about 50 atoms, e.g., between 1 and 40, between 1 and 30, between 1 and 20, between 1 and 10, or between 1 and 6 atoms, where length refers to the number of atoms in the main (longest) chain. In some embodiments, L comprises two or more different moieties linked to one another to form a structure typically having between 1 to about 40, e.g., between 1 and 30, e.g., between 1 and 20, between 1 and 10, or between 1 and 6 carbon atoms in the main (longest) chain.

In some embodiments a polypeptide C5L2 modulator is extended by one or more amino acids at the N-terminus, C-terminus, or both, wherein at least one of the amino acids has a side chain that comprises a reactive functional group such as a primary or secondary amine, a sulfhydryl group, a carboxyl group (which may be present as a carboxylate group), a guanidino group, a phenol group, an indole ring, a thioether, or an imidazole ring, wherein the reactive functional group may be used, e.g., to attach a moiety. In some embodiments, the amino acid(s) is/are L-amino acids. In some embodiments, any one or more of the amino acid(s) is a D-amino acid. If multiple amino acids are added, the amino acids can be independently selected. In some embodiments, the reactive functional group (e.g., a primary or secondary amine) is used as a target for addition of a moiety. Amino acids having a side chain that comprises a primary or secondary amine include lysine (Lys) and diaminocarboxylic acids of general structure NH₂(CH₂)ₙCH(NH₂)COOH such as 2,3-diaminopropionic acid (dapa), 2,4-diaminobutyric acid (daba), and ornithine (orn), wherein n=1 (dapa), 2 (daba), and 3 (orn), respectively. A wide variety of non-standard amino acids having side chains that comprise one or more such reactive functional group(s) are available, including naturally occurring amino acids and amino acids not found in nature. See, e.g., Hughes, B. (ed.), Amino Acids, Peptides and Proteins in Organic Chemistry, Volumes 1-4, Wiley-VCH (2009-2011); Blaskovich, M., Handbook on Syntheses of Amino Acids General Routes to Amino Acids, Oxford University Press, 2010. Embodiments in which one or more non-standard amino acid(s) is/are used to provide a target for addition of a moiety are encompassed. Any one or more of the amino acid(s) may be protected as appropriate during synthesis of the compound. For example, one or more amino acid(s) may be protected during reaction(s) involving the target amino acid side chain. In some embodiments, wherein a sulfhydryl-containing amino acid is used as a target for addition of a moiety comprising a CRM, the sulfhydryl is protected while the compound is being cyclized by formation of an intramolecular disulfide bond between other amino acids such as cysteines.

In some embodiments, at least one reactive functional group is introduced into the polypeptide. For example, in some embodiments at least one side chain of the polypeptide is modified to convert a first reactive functional group to a different reactive functional group prior to reaction with the compstatin analog. In some embodiments a thiol is introduced. Several methods are available for introducing thiols into biomolecules, including the reduction of intrinsic disulfides, as well as the conversion of amine, aldehyde or carboxylic acid groups to thiol groups. Disulfide crosslinks of cystines in proteins can be reduced to cysteine residues by dithiothreitol (DTT), tris-(2-carboxyethyl)phosphine (TCEP), or tris-(2-cyanoethyl)phosphine. Amines can be indirectly thiolated by reaction with succinimidyl 3-(2-pyridyldithio)propionate (SPDP) followed by reduction of the 3-(2-pyridyldithio)propionyl conjugate with DTT or TCEP. Amines can be indirectly thiolated by reaction with succinimidyl acetylthioacetate followed by removal of the acetyl group with 50 mM hydroxylamine or hydrazine at near-neutral pH. Amines can be directly thiolated by reaction with 2-iminothiolane, which preserve the overall charge of the molecule and introduces a free thiol. Tryptophan residues in thiol-free proteins can be oxidized to mercaptotryptophan residues, which can then be modified by iodoacetamides or maleimides. A polypeptide comprising one or more thiols may be reacted with a compound comprising a maleimide group.

In some aspects, nucleic acids comprising a sequence that encodes any of the polypeptide C5L2 modulators are provided. For example, a polypeptide comprising a C5a variant, which in some embodiments is fused (either directly or via a linker polypeptide) to a cell-penetrating peptide, targeting polypeptide, cell membrane binding polypeptide, or polypeptide that serves as a clearance reducing moiety, are provided. Also provided are nucleic acids encoding an antibody or non-antibody engineered polypeptide that binds to C5 (e.g., the C5 alpha chain) or that binds specifically to C5a (and not to intact C5), which in some embodiments is fused (either directly or via a linker polypeptide) to a cell-penetrating peptide, targeting polypeptide, cell membrane binding polypeptide, or polypeptide that serves as a clearance reducing moiety, are provided. In certain embodiments a polypeptide is encoded by an open reading frame. Also provided are vectors comprising the nucleic acid. In some embodiments the nucleic acid encoding a C5L2 modulator is operably linked to expression control elements appropriate to direct expression in prokaryotic or eukaryotic cells. Also provided are prokaryotic and eukaryotic cells comprising such nucleic acids or vectors. In certain embodiments such cells may be used to produce the C5L2 modulator.

### IV. Screening Methods

In some aspects, the disclosure provides methods of identifying candidate modulators of C5L2. Certain of the methods comprise contacting a mammalian T cell or monocyte with a test agent and determining whether the test agent increases or decreases production of IL-17, IFN-γ, IL-6, IL-1β, or a combination thereof. In some embodiments a method of identifying a candidate inhibitor of C5L2 comprises contacting a mammalian T cell or monocyte with a test agent and determining whether the test agent increases production of IL-6, IL-1β, or both, by the T cell, wherein an agent that increases production of IL-6, IL-1β, or both, by the T cell or monocyte is a candidate inhibitor of C5L2. A method of identifying a candidate inhibitor of C5L2, the method comprising contacting a mammalian T cell with a test agent and determining whether the test agent increases production of IL-17, IFN-γ, or both, by the T cell, wherein an agent that increases production of IL-17, IFN-γ, or both, by the T cell, is a candidate inhibitor of C5L2.

In some embodiments a method of identifying a candidate activator of C5L2 comprises contacting a mammalian T cell with a test agent and determining whether the test agent decreases production of IL-17, IFN-γ, or both, by the T cell, wherein an agent that decreases production of IL-17, IFN-γ, or both, by the T cell, is a candidate activator of C5L2. In some embodiments a method of identifying a candidate activator of C5L2 comprises contacting a mammalian T cell, e.g., a CD4+ T cell, with a test agent and determining whether the test agent decreases production of IL-6, IL-1β, or both, by the T cell or monocyte, wherein an agent that decreases production of IL-6, IL-1β, or both, by the T cell or monocyte, is a candidate activator of C5L2.

In some embodiments a method of identifying a candidate inhibitor of C5L2, the method comprises contacting a mammalian nTreg cell with a test agent and determining whether the test agent increases or decreases suppressive activity of the nTreg cell, wherein an agent that decreases suppressive activity of the nTreg cell is a candidate activator or inhibitor of C5L2, respectively.

In certain embodiments a method comprises identifying an agent that binds to C5L2 or a fragment thereof. In some embodiments the agent may be tested using any of the afore-mentioned assays to evaluate its activity as an activator or inhibitor of C5L2.

In some embodiments a method comprises assessing the ability of a test agent to inhibit intracellular cleavage of C5, e.g., intracellular production of C5a, by a T cell or monocyte and/or identifying an agent capable of inhibiting intracellular cleavage of C5, e.g., intracellular production of C5a, by a T cell or monocyte.

Any of a wide variety of agents may be used as test agents may be used in various embodiments. For example, a test agent may be a small molecule, polypeptide, peptide, nucleic acid, oligonucleotide, lipid, carbohydrate. Agents can be obtained from natural sources or produced synthetically. Agents may be at least partially pure or may be present in extracts or other types of mixtures. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. The library may comprise, e.g., between 100 and 500,000 compounds, or more. Compounds are often arrayed in multwell plates. They may be dissolved in a solvent (e.g., DMSO) or provided in dry form, e.g., as a powder or solid. Collections of synthetic, semi-synthetic, and/or naturally occurring compounds may be tested. Compound libraries can comprise structurally related, structurally diverse, or structurally unrelated compounds. Compounds may be artificial (having a structure invented by man and not known to be found in nature) or naturally occurring. In some embodiments a library comprises at least some compounds that have been identified as “hits” or “leads” in a drug discovery program and/or analogs thereof. A compound library may comprise natural products and/or compounds generated using non-directed or directed synthetic organic chemistry. A compound library may be a small molecule library. Other libraries of interest include peptide or peptoid libraries, ORF libraries, cDNA libraries, and oligonucleotide libraries. A library may be focused (e.g., composed primarily of compounds having the same core structure, derived from the same precursor, or having at least one biochemical activity in common). Compound libraries are available from a number of commercial vendors such as Tocris BioScience, Nanosyn, BioFocus, and from government entities.

### V. Methods of Treatment and Compositions of Use Therefor

In some aspects, the disclosure provides methods of treating any of a variety of disorders, the methods comprising administering a C5L2 modulator to a subject in need of treatement for the disorder. In some embodiments a disorder treated according to the methods is a chronic disorder. In various embodiments the C5L2 modulator may be any of the C5L2 modulators described herein.

In some embodiments a disorder treated according to the methods, e.g., a chronic disorder, is a Th1 disorder. “Th1 disorder” refers to any disorder characterized in that CD4+T lymphocytes of the Th1 subtype (“Th1 cells”) contribute to its pathogenesis, progression, or severity and/or that is characterized by an excessive number and/or excessive or inappropriate activity of Th1 cells in the body or a portion thereof, e.g., in at least one body fluid, tissue, organ, or structure. For example, there may be an excessive number and/or excessive or inappropriate activity of Th1 cells in the blood and/or in at least one tissue, organ, or structure affected by a disorder. Th1 disorders include those in which excessive or inappropriate levels of one or more Th1 cytokines contribute to tissue damage or dysfunction or other deleterious effects. In some embodiments an excessive number of Th1 cells is a relative predominance, e.g., the ratio of Th1 cells to Th2 cells and/or the ratio of Th1 cells to Th17 cells, is increased relative to normal values. In some embodiments a Th1 disorder is acute transplant rejection. In some aspects, a subject with a Th1 disorder would benefit from a decreased Th1 response. In some embodiments a decreased Th1 response is a reduction to a normal value. In some embodiments, a method comprises administering a C5L2 activator to a subject who may benefit from a reduction in Th1 response.

In some embodiments a disorder treated according to the methods, e.g., a chronic disorder, is a Th17 disorder. “Th17 disorder” refers to any disorder characterized in that CD4+ T lymphocytes of the Th17 subtype (“Th17 cells”) contribute to its pathogenesis, progression, or severity and/or that is characterized by an excessive number and/or excessive or inappropriate activity of Th17 cells in the body or a portion thereof, e.g., in at least one body fluid, tissue, organ, or structure. For example, there may be an excessive number and/or excessive or inappropriate activity of Th17 cells in the blood and/or in at least one tissue, organ, or structure affected by a disorder. Th17 disorders include those in which excessive or inappropriate levels of one or more Th17 cytokines contribute to tissue damage or dysfunction or other deleterious effects. In some embodiments an excessive number of Th17 cells is a relative predominance, e.g., the ratio of Th17 cells to Th1 cells and/or the ratio of Th17 cells to Th2 cells, is increased relative to normal values. In some aspects, a subject with a Th17 disorder would benefit from a decreased Th17 response. In some embodiments a decreased Th17 response is a reduction to a normal value. In some embodiments, a method comprises administering a C5L2 activator to a subject who may benefit from a reduction in Th17 response.

In some embodiments a disorder treated according to the methods, e.g., a chronic disorder, is an IL-6 mediated disorder. The term “IL-6 mediated disorder” refers to any disorder characterized in that IL-6 contributes to its pathogenesis, progression, or severity or to one or more of its symptoms. In some aspects, an IL-6 mediated disorder is characterized by an abnormally high level of IL-6 and/or an abnormally high level of IL-6 secreting cells in the blood and/or in one or more tissues or organs that manifests symptoms of the disorder in subjects who have the disorder as compared with normal subjects who do not have the disorder. In some aspects, an IL-6 mediated disorder is characterized by an abnormally high level of IL-6 signaling in one or more cell types or subtypes and/or in one or more tissues or organs that manifests symptoms of the disorder, or both, in subjects who have the disorder as compared with normal subjects who do not have the disorder. In some aspects, an IL-6 mediated disorder is a disorder for which at least one anti IL-6 agent has demonstrated efficacy as a treatment in at least one controlled, randomized clinical trial. In some aspects, an IL-6 mediated disorder is a disorder for which at least one anti IL-6 agent has demonstrated efficacy as a treatment in at least one Phase II or Phase III clinical trial. In some aspects, an IL-6 mediated disorder is characterized in that at least one anti IL-6 agent has been approved by the US Food & Drug Administration, European Medicines Agency, or both, as a treatment for the disorder. In some aspects, an IL-6 mediated disorder is characterized in that those of ordinary skill in the art consider an anti IL-6 agent to be an appropriate treatment, e.g., an accepted off-label use, for at least some subjects suffering from the disorder. Anti-IL-6 agents include, e.g., antibodies and polypeptides that bind to the IL-6 receptor (e.g., tocilizumab, sarilumab), and antibodies and polypeptides that bind to IL-6 (e.g., sirukumab, olokizumab, siltuximab, BMS-945429). In some embodiments an IL-6 mediated disorder is an inflammatory rheumatic disease (e.g., rheumatoid arthritis (RA), juvenile idiopathic arthritis, polymyalgia rheumatica, relapsing polychondritis, spondyloarthritides such reactive arthritis, ankylosing spondylitis (AS), psoriatic arthritis, inflammatory bowel disease-related arthritis and undifferentiated spondyloarthropathy), scleroderma, systemic lupus erythematosus (SLE), systemic sclerosis, Crohn's disease, adult onset Still's disease, vasculitis (e.g., Takayasu arteritis, giant cell arteritis), inflammatory myopathy (e.g., polymyositis (PM), dermatomyositis, inclusion body myositis), relapsing polychondritis multiple sclerosis, neuromyelitis optica, Behcet's disease, or uveitis. In some embodiments an IL-6 mediated disorder is characterized by an excessive number of and/or excessive proliferation of cells whose survival and/or proliferation is enhanced by IL-6 or that are descendants of such cells. For example, in some embodiments an IL-6 mediated disorder is characterized by an excessive number of and/or excessive proliferation of B cells or plasma cells. For example, an IL-6 mediated disease may be multiple myeloma, a B cell lymphoma, or Castleman's disease. In some embodiments, a method comprises administering a C5L2 activator to a subject with an IL-6 mediated disease.

In some embodiments a disorder treated according to the methods, e.g., a chronic disorder, is an IL-1β mediated disorder. The term “IL-1β mediated disorder” refers to any disorder characterized in that IL-1β contributes to its pathogenesis, progression, or severity or to one or more of its symptoms. In some aspects, an IL-1β mediated disorder is characterized by an abnormally high level of IL-1β and/or an abnormally high level of IL-1β secreting cells in the blood and/or in one or more tissues or organs that manifests symptoms of the disorder in subjects who have the disorder as compared with normal subjects who do not have the disorder. In some aspects, an IL-1β mediated disorder is characterized by an abnormally high level of IL-1β signaling in one or more cell types or subtypes and/or in one or more tissues or organs that manifests symptoms of the disorder, or both, in subjects who have the disorder as compared with normal subjects who do not have the disorder. In some aspects, an IL-1β mediated disorder is a disorder for which at least one anti IL-1β agent has demonstrated efficacy as a treatment in at least one controlled, randomized clinical trial. In some aspects, an IL-1β mediated disorder is a disorder for which at least one anti IL-1β agent has demonstrated efficacy as a treatment in at least one Phase II or Phase III clinical trial. In some aspects, an IL-1β mediated disorder is characterized in that at least one anti IL-1β agent has been approved by the US Food & Drug Administration, European Medicines Agency, or both, as a treatment for the disorder. In some aspects, an IL-1β mediated disorder is characterized in that those of ordinary skill in the art consider an anti IL-1β agent to be an appropriate treatment, e.g., an accepted off-label use, for at least some subjects suffering from the disorder. Anti IL-1β agents include antibodies against IL-1β (such as canakinumab), other IL-1β binding proteins (such as rilonacept), and IL-1β receptor antagonists (for example anakinra). In some embodiments, a method comprises administering a C5L2 activator to a subject with an IL-1β mediated disorder.

In some embodiments an IL-1β mediated disorder is a cryopyrin-associated periodic syndrome (CAPS). CAPS is a spectrum of autoinflammatory syndromes including familial cold autoinflammatory syndrome (FCAS, formerly termed familial cold-induced urticaria), the Muckle-Wells syndrome (MWS), and neonatal-onset multisystem inflammatory disease (NOMID, also called chronic infantile neurologic cutaneous and articular syndrome or CINCA) that share many clinical features. (Kubota T, Koike R. (2010) Mod Rheumatol. 20(3):213-21). These syndromes are associated with mutations in NLRP3, the gene encoding cryopyrin, a component of the inflammasome, and mutations lead to unregulated production of interleukin 1β. Canakinumab, rilonacept, or anakinra can be used to treat these disorders.

In some embodiments a disorder treated according to the methods, e.g., a chronic disorder, is characterized by an abnormal number and/or abnormal functional activity of Tregs, e.g., an abnormal number and/or abnormal activity of nTregs. In some embodiments a disorder, e.g., a chronic disorder, is characterized by a deficiency or lack of functional activity of Tregs, e.g., nTregs. Deficiency or lack of functional activity of Tregs is implicated, for example, in the occurrence, progression, and/or persistence of autoimmune diseases and inflammatory disorders. Deficiency or lack of functional activity of Tregs may permit or enhance the generation of and/or at least in part prevent the shutdown of effector immune cells (e.g., CD4+ cells, CD8+ T cells, B cells) that would otherwise limit the immune response or that would otherwise inhibit an immune response against “self” antigens. In certain embodiments, autoimmune disorders, inflammatory disorders, or other disorders that may be associated with a deficiency, decreased number, and/or lack of functional activity of Tregs may be treated with a C5L2 activator.

In some embodiments a disorder, e.g., a chronic disorder, is characterized by an excessive number and/or excessive functional activity of Tregs, e.g., nTregs. Increased number and/or excessive functional activity of Tregs has been implicated, for example, in inhibiting immune system attack on cancer cells, pathogens, or pathogen-infected cells, thus contributing to the occurrence, progression, and/or persistence of cancer and/or infections. In certain embodiments, cancer, infections, or other disorders that may be associated with an excessive number and/or increased functional activity of Tregs may be treated with a C5L2 inhibitor. In certain embodiments treatment with a C5L2 inhibitor may inhibit generation of Tregs that may otherwise limit the efficacy of a vaccine or immune response. In accordance with certain embodiments a C5L2 inhibitor may be used as a component of or in combination with a vaccine or cell-based immunotherapy for, e.g., cancer or an infectious disease.

It will be understood that a disease may fall into one or more than one of the categories described herein. For example, a disease may be a Th17 disorder and an IL-6 mediated disease. Any disease may, in addition to being a Th1 disorder, Th17 disorder, and/or IL-6 mediated disease, disease, also be associated with an abnormal number and/or abnormal functional activity of Tregs, e.g., an abnormal number and/or abnormal activity of nTregs.

In some embodiments, an autoimmune disease or inflammatory disease is characterized by the presence of autoantibodies and/or immune complexes in the body.

In some embodiments, a chronic disorder that may be treated using a C5L2 activator is a respiratory disorder. In some embodiments, the chronic respiratory disorder is asthma or chronic obstructive pulmonary disease (COPD). In some embodiments, a chronic respiratory disorder is pulmonary fibrosis (e.g., idiopathic pulmonary fibrosis), radiation-induced lung injury, allergic bronchopulmonary aspergillosis, hypersensitivity pneumonitis (also known as allergic alveolitis), eosinophilic pneumonia, interstitial pneumonia, sarcoid, Wegener's granulomatosis, or bronchiolitis obliterans.

In some embodiments, a chronic disorder treated that may be treated using a C5L2 activator is a disorder that affects the musculoskeletal system. Examples of such disorders include inflammatory joint conditions (e.g., arthritis such as rheumatoid arthritis or psoriatic arthritis, juvenile chronic arthritis, spondyloarthropathies Reiter's syndrome, gout). In some embodiments, a musculoskeletal system disorder results in symptoms such as pain, stiffness and/or limitation of motion of the affected body part(s). Inflammatory myopathies include dermatomyositis, polymyositis, and various others are disorders of chronic muscle inflammation of unknown etiology that result in muscle weakness. In some embodiments, a chronic disorder is myasthenia gravis.

In some embodiments, a chronic disorder that may be treated using a C5L2 activator is is a disorder that affects the integumentary system. Examples of such disorders include, e.g., atopic dermatitis, psoriasis, pemphigus, systemic lupus erythematosus, dermatomyositis, scleroderma, sclerodermatomyositis, Sjögren syndrome, and chronic urticaria.

In some embodiments, a chronic disorder affects the nervous system, e.g., the central nervous system (CNS) and/or peripheral nervous system (PNS). Examples of such disorders include, e.g., multiple sclerosis, other chronic demyelinating diseases, amyotrophic lateral sclerosis, chronic pain, stroke, allergic neuritis, Huntington's disease, Alzheimer's disease, and Parkinson's disease. In some embodiments such disorder is associated with CNS inflammation.

In some embodiments, a chronic disorder that may be treated using a C5L2 activator affects the circulatory system. For example, in some embodiments the disorder is a vasculitis or other disorder associated with vessel inflammation, e.g., blood vessel and/or lymph vessel inflammation. In some embodiments, a vasculitis is polyarteritis nodosa, Wegener's granulomatosis, giant cell arteritis, Churg-Strauss syndrome, microscopic polyangiitis, Henoch-Schonlein purpura, Takayasu's arteritis, Kawasaki disease, or Behcet's disease. In some embodiments, a subject, e.g., a subject in need of treatment for vasculitis, is positive for antineutrophil cytoplasmic antibody (ANCA).

In some embodiments, a chronic disorder that may be treated using a C5L2 activator is affects the gastrointestinal system. For example, the disorder may be inflammatory bowel disease, e.g., Crohn's disease or ulcerative colitis.

In some embodiments, a chronic disorder that may be treated using a C5L2 activator is is a thyroiditis (e.g., Hashimoto's thryoiditis, Graves' disease, post-partum thryoiditis), myocarditis, hepatitis (e.g., hepatitis C), pancreatitis, glomerulonephritis (e.g., membranoproliferative glomerulonephritis or membranous glomerulonephritis), or panniculitis.

In some embodiments, a chronic disorder that may be treated using a C5L2 activator is is a chronic eye disorder. In some embodiments, the chronic eye disorder is characterized by macular degeneration, choroidal neovascularization (CNV), retinal neovascularization (RNV), ocular inflammation, or any combination of the foregoing. Macular degeneration, CNV, RNV, and/or ocular inflammation may be a defining and/or diagnostic feature of the disorder. Exemplary disorders that are characterized by one or more of these features include, but are not limited to, macular degeneration related conditions, diabetic retinopathy, retinopathy of prematurity, proliferative vitreoretinopathy, uveitis, keratitis, conjunctivitis, and scleritis. Macular degeneration related conditions include, e.g., age-related macular degeneration (AMD). In some embodiments, a subject is in need of treatment for wet AMD. In some embodiments, a subject is in need of treatment for dry AMD. In some embodiments, a subject is in need of treatment for geographic atrophy (GA). In some embodiments, a subject is in need of treatment for ocular inflammation. Ocular inflammation can affect a large number of eye structures such as the conjunctiva (conjunctivitis), cornea (keratitis), episclera, sclera (scleritis), uveal tract, retina, vasculature, and/or optic nerve. Evidence of ocular inflammation can include the presence of inflammation-associated cells such as white blood cells (e.g., neutrophils, macrophages) in the eye, the presence of endogenous inflammatory mediator(s), one or more symptoms such as eye pain, redness, light sensitivity, blurred vision and floaters, etc. Uveitis is a general term that refers to inflammation in the uvea of the eye, e.g., in any of the structures of the uvea, including the iris, ciliary body or choroid. Specific types of uveitis include iritis, iridocyclitis, cyclitis, pars planitis and choroiditis. In some embodiments, a subject is in need of treatment for geographic atrophy (GA). In some embodiments, the chronic eye disorder is an eye disorder characterized by optic nerve damage (e.g., optic nerve degeneration), such as glaucoma.

In some embodiments a disorder that may be treated using a C5L2 activator is acute rejection of a transplanted organ, tissue, cells or populations of cells. As used herein, “acute rejection” refers to rejection occurring typically within 6 months post-transplant though it can occur later, e.g., if a subject ceases using an immunosuppressive therapy. In some embodiments acute rejection occurs at least 1, 2, or 3 days post-transplant and/or is not hyperacute rejection. In some embodiments, a chronic disorder that may be treated using a C5L2 activator is is chronic rejection of a transplanted organ, tissue, cells or populations of cells (collectively “grafts”). Examples of grafts include, e.g., solid organs such as kidney, liver, lung, pancreas, heart; tissues such as cartilage, tendons, cornea, skin, heart valves, and blood vessels; pancreatic islets or islet cells. Transplant rejection is one of the major risks associated with transplants between genetically different individuals of the same species (allografts) or between individuals of different species (xenografts) and can lead to graft failure and a need to remove the graft from the recipient. As used herein, “chronic rejection” refers to rejection occurring typically at least 6 months post-transplant, e.g., between 6 months and 1, 2, 3, 4, 5 years, or more post-transplant, often after months to years of good graft function. For purposes hereof, chronic rejection can include chronic graft vasculopathy, a term used to refer to fibrosis of the internal blood vessels of the transplanted tissue. In some embodiments, one or more cells, tissues, or organs is contacted with a C5L2 modulator ex vivo (outside the body of a subject). In some embodiments the cell, tissue, or organ is to be transplanted into a subject. In some embodiments a disorder that may be treated using a C5L2 activator is graft-versus-host disease.

In some embodiments a cell, tissue, or organ to be introduced into a subject for transplantation or cell-based immunotherapy originated from the subject (autologous). In some embodiments a cell, tissue, or organ to be introduced into a subject originated from a different subject (donor) of the same species. In some embodiments the donor is a histocompatible to the subject within art-accepted guidelines for transplantation. In some embodiments a cell introduced or to be introduced into a subject, e.g., as a transplant or immunotherapy, originated from a different species, e.g., a transplant is a xenograft.

In some embodiments, a method comprises administering one or more doses of a C5L2 modulator, e.g., a C5L2 activator, to a subject in need thereof, in an amount sufficient to reduce and/or maintain concentration of one or more cytokines and/or concentration of one or more T cell subsets towards or to within the normal range. In some embodiments, one or more doses sufficient to reduce and/or maintain concentration of one or more cytokines and/or concentration of one or more T cell subsets in a tissue or organ affected by a disorder towards or to within the normal range is administered. The subject in need thereof may have abnormally high levels of the cytokine and/or abnormally or undesirably high levels or activity of the T cell subset.

In some embodiments, a method comprises administration of one or more doses of a C5L2 modulator, e.g., a C5L2 inhibitor, to a subject in need thereof, in an amount sufficient to increase and/or maintain concentration of one or more cytokines and/or concentration of one or more T cell subsets towards or to within the normal range. In some embodiments, one or more doses sufficient to increase and/or maintain concentration of one or more cytokines and/or concentration of one or more T cell subsets in a tissue or organ affected by a disorder towards or to within the normal range is administered. The subject in need thereof may have abnormally low levels of the cytokine and/or abnormally low levels or activity of the T cell subset.

In some aspects, “normal range” refers to a range of within ±2 standard deviations from a mean value (e.g., an arithmetic mean value) in a population of subjects (e.g., healthy subjects) and/or the range into which at least 95% of subjects, e.g., at least 95% of healthy subjects, fall. One of ordinary skill in the art will appreciate that the specific values for a “normal range” may at least in part depend on the particular assay used to assess a parameter of interest and/or factors such as the specific reagents used. In some embodiments, a normal range may be determined using published data. In some embodiments, a normal range may be appropriately defined by a laboratory, testing center, ordinary skilled artisan, etc. It will also be understood that a normal range may be adjusted for demographic variables such as age, gender, etc., where appropriate.

The term “cancer” is generally used herein and/or to refer to a disease characterized by one or more tumors, e.g., one or more benign, malignant, or potentially malignant abnormal growths comprising aberrantly proliferating cells. Cancer includes, but is not limited to: breast cancer; biliary tract cancer; bladder cancer; brain cancer (e.g., glioblastomas, medulloblastomas); cervical cancer; choriocarcinoma; colon cancer; endometrial cancer; esophageal cancer; gastric cancer; hematological neoplasms including acute lymphocytic leukemia and acute myelogenous leukemia; T-cell acute lymphoblastic leukemia/lymphoma; hairy cell leukemia; chronic lymphocytic leukemia, chronic myelogenous leukemia, multiple myeloma; adult T-cell leukemia/lymphoma; intraepithelial neoplasms including Bowen's disease and Paget's disease; liver cancer; lung cancer; lymphomas including Hodgkin's disease and lymphocytic lymphomas; neuroblastoma; melanoma, oral cancer including squamous cell carcinoma; ovarian cancer including ovarian cancer arising from epithelial cells, stromal cells, germ cells and mesenchymal cells; neuroblastoma, pancreatic cancer; prostate cancer; rectal cancer; sarcomas including angiosarcoma, gastrointestinal stromal tumors, leiomyosarcoma, rhabdomyosarcoma, liposarcoma, fibrosarcoma, and osteosarcoma; renal cancer including renal cell carcinoma and Wilms tumor; skin cancer including basal cell carcinoma and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullary carcinoma. It will be appreciated that a variety of different tumor types can arise in certain organs, which may differ with regard to, e.g., clinical and/or pathological features and/or molecular markers. Tumors arising in a variety of different organs are discussed, e.g., in DeVita, supra or in the WHO Classification of Tumours series, 4^{th} ed, or 3^{rd} ed (Pathology and Genetics of Tumours series), by the International Agency for Research on Cancer (IARC), WHO Press, Geneva, Switzerland, all volumes of which are incorporated herein by reference.

“Infection” or “infectious disease” encompasses any disorder caused by an infectious agent such as virus, bacterium, fungus (e.g., mold or yeast), protozoa, or multicellular parasite. One of ordinary skill in the art will be aware of numerous microbes (e.g., viruses, bacteria, fungi, protozoa) and multicellular parasites capable of causing disease in mammals. Exemplary viruses of interest include, e.g., Retroviridae (e.g., lentiviruses such as human immunodeficiency viruses, such as HIV-I); Caliciviridae (e.g. strains that cause gastroenteritis); Togaviridae (e.g. equine encephalitis viruses, rubella viruses); Flaviridae (e.g. dengue viruses, encephalitis viruses, yellow fever viruses, hepatitis C virus); Coronaviridae (e.g. coronaviruses); Rhabdoviridae (e.g. vesicular stomatitis viruses, rabies viruses); Filoviridae (e.g. Ebola viruses); Paramyxoviridae (e.g. parainfluenza viruses, mumps virus, measles virus, respiratory syncytial virus); Orthomyxoviridae (e.g. influenza viruses); Bunyaviridae (e.g. Hantaan viruses, bunga viruses, phleboviruses and Nairo viruses); Arenaviridae (hemorrhagic fever viruses); Reoviridae (erg., reoviruses, orbiviurses and rotaviruses); Birnaviridae; Hepadnaviridae (Hepatitis B or C virus); Parvoviridae (parvoviruses); Papovaviridae (papilloma viruses, polyoma viruses); Adenoviridae; Herpesviridae (herpes simplex virus (HSV) 1 and 2, varicella zoster virus, cytomegalovirus (CMV), EBV, KSV); Poxviridae (variola viruses, vaccinia viruses, pox viruses); and Picornaviridae (e.g. polio viruses, hepatitis A virus; enteroviruses, human coxsackie viruses, rhinoviruses, echoviruses).

Bacteria of interest include, e.g., gram positive, gram negative, and acid-fast bacteria. Bacteria may be cocci, rod-shaped, spirochetes. Exemplary bacteria include, e.g., Helicobacter pylori, Borellia (e.g., B. burgdorferi), Legionella pneumophilia, Mycobacteria (e.g., M. tuberculosis, M. avium, M. intracellulare, M. kansasii, M. gordonae), Staphylococcus (e.g., Staphylococcus aureus), Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, Campylobacter sp., Enterococcus sp., Chlamydia sp., Haemophilus influenzae, Bordetella (e.g., B. pertussis, B. parapertussis), Bacillus anthraces, Corynebacterium diphtheriae, Clostridia (e.g., Clostridium perfringens, Clostridium tetani, Clostridium difficile), Enterobacter aerogenes, Pseudomonas, Klebsiella pneumoniae, Proteus, Enterobacter, Serratia, Citrobacter, Bacteroides sp., Treponema pallidum, Leptospira, Actinomyces israelii, Francisella tularensis, Salmonella, Shigella, and E. coli (e.g., pathogenic E. coli).

In some embodiments a fungus is a member of the phylum Ascomycota, Basidiomycota, Chytridiomycota, Glomeromycota, or Zygomycota. The fungus may be a member of a genus selected from the group consisting of Aspergillus, Blastomyces, Candida, Coccidioides, Cryptococcus, Epidermophytum, Exserohilum, Fusarium, Histoplasma, Malassezia, Microsporum, Mucor, Paracoccidioides, Penicillium, Pichia, Pneumocystis, Pseudallescheria, Rhizopus, Rhodotorula, Scedosporium, Schizophyllum, Sporothrix, Stachybotrys, Saccharomyces, Trichophyton, Trichosporon, Bipolaris, Exserohilum, Curvularia, Alternaria, or Cladophialophora. Exemplary fungi include, e.g., Aspergillus, such as Aspergillus flavus, Aspergillus fumigatus, Aspergillus niger; Candida, such as Candida albicans, Candida glabrata, Candida guilliermondii, Candida krusei, Candida parapsilosis, Candida tropicalis, Coccidioides, such as Coccidioides immitis, Cryptococcus, such as Cryptococcus neoformans, Histoplasma, such as Histoplasma capsulatum, or Coccidioides immitis.

In some embodiments a parasite is a protozoan. In some embodiments the parasite belongs to the phylum Apicomplexa. Exemplary parasites include, e.g., parasites of the genus Plasmodium (Plasmodium falciparum, Plasmodium vivax, Plasmodium ovale curtisi, Plasmodium ovale wallikeri, Plasmodium malariae, or Plasmodium knowlesi), Trypanosoma, Toxoplasma (e.g., Toxoplasma gondii), Babesia, Leishmania (e.g., Leishmania major), Isospora, Schistosoma, or Cryptosporidium. In some embodiments a protozoan is a kinetoplastid. In some embodiments a kinetoplastid is a trypanosomatid, e.g., a member of the genus Leishmania, e.g., L. donovani, L. major, L. tropica, or L. braziliensis, or a member of the genus Trypanosoma, e.g., T. brucii, T. cruzii, T. congolense, or T. equiperdum.

In some embodiments a parasite resides extracellularly during at least part of its life cycle. Examples include nematodes, trematodes (flukes), and cestodes. In some embodiments an antigen may be from a nematode such as Ascaris, Enterobius, Thichuris, and/or cestodes such as Taenia, Hymenolepis, and Echinococcus, a cestode such as Taenia, Hymenolepis, Echinococcus, or Fasciola, a trematode such as Schistosoma. In some embodiments a parasite is Trichinella, Diphyllobothrium, Clonorchis, Paragonimus, Ancylostoma, Necator, Strongyloides, Wuchereria, Onchocerca, or Dracunculus.

As noted above, in certain embodiments a C5L2 inhibitor may be used as a component of or in combination with a vaccine or cell-based immunotherapy for, e.g., cancer or an infectious disease. In general, a vaccine may comprise one or more antigens against which an immune response is desired. Other vaccine components which may be present include any of a variety of adjuvants. The term “adjuvant” encompasses substances that accelerate, prolong, or enhance the immune response to an antigen. An adjuvant may serve as a lymphoid system activator that enhances the immune response in a relatively non-specific manner, e g., without having any specific antigenic effect itself. In certain embodiments an adjuvant enhances antigen-specific immune responses when used in combination with a specific antigen or antigens, e.g., as a component of a vaccine. Adjuvants include, but are not limited to, aluminum salts (alum) such as aluminum hydroxide or aluminum phosphate, complete Freund's adjuvant, incomplete Freund's adjuvant, surface active substances such as lysolecithin, pluronic polyols, Amphigen, Avridine, bacterial lipopolysaccharides, 3-O-deacylated monophosphoryl lipid A, synthetic lipid A analogs or aminoalkyl glucosamine phosphate compounds (AGP), or derivatives or analogs thereof (see, e.g., U.S. Pat. No. 6,113,918), L121/squalene, muramyl dipeptide, polyanions, peptides, saponins, oil or hydrocarbon and water emulsions, particles such as ISCOMS (immunostimulating complexes), CD40 agonist, anti-CD40 antibody, CD40 ligand, such as CD40L, a ligand for a Toll-like receptor (TLR). In certain embodiments a C5L2 inhibitor may serve as an adjuvant.

In some embodiments a vaccine is administered prophylactically, to a subject who is apparently healthy. In some embodiments a vaccine is administered to a subject who has a disease, e.g., the subject shows symptoms or signs of an infection or cancer and the vaccine is intended to treat such infection or cancer. In some embodiments a vaccine may be administered in an effort to reduce the likelihood of recurrence of an infection or cancer.

A vaccine for cancer may comprise one or more tumor antigen (TAs). In general, a tumor antigen can be any antigenic substance produced by cells in a tumor, e.g., tumor cells or in some embodiments tumor stromal cells (e.g., tumor-associated cells such as cancer-associated fibroblasts). In certain embodiments a TA is a molecule (or portion thereof) that is expressed at higher levels by cancer cells as compared with non-cancer cells. A TA may be expressed by a subset of cancers of a particular type and/or by a subset of cells in a tumor. A TA may at least in part exposed at the cell surface of tumor cells or tumor stromal cells. In some embodiments a TA comprises an abnormally modified protein, lipid, glycoprotein, or glycolipid. Tumor antigens may include, e.g., proteins that are normally produced in very small quantities and are expressed in larger quantities by tumor cells, proteins that are normally produced only in certain stages of development, proteins whose structure (e.g., sequence or post-translational modification(s)) is modified due to mutation in tumor cells, or normal proteins that are (under normal conditions) sequestered from the immune system. In some embodiments a TA is an expression product of a mutated gene, e.g., an oncogene or mutated tumor suppressor gene, an overexpressed or aberrantly expressed cellular protein, an antigen encoded by an oncogenic virus (e.g., HBV; HCV; herpesvirus family members such as EBV, KSV; papilloma virus, etc.), or an oncofetal antigen. Oncofetal antigens are normally produced in the early stages of embryonic development and largely or completely disappear by the time the immune system is fully developed. Examples are alphafetoprotein (AFP, found, e.g., in germ cell tumors and hepatocellular carcinoma) and carcinoembryonic antigen (CEA, found, e.g., in bowel cancers and occasionally lung or breast cancer). Tyrosinase is an example of a protein normally produced in very low quantities but whose production is greatly increased in certain tumor cells (e.g., melanoma cells). TAs include, e.g., CA-125 (found, e.g., in ovarian cancer); MUC-1 (found, e.g., in breast cancer); HER-2/neu (found, e.g., in breast cancer); melanoma-associated antigen (MAGE; found, e.g., in malignant melanoma); prostatic acid phosphatase (PAP, found in prostate cancer), Wilms' tumor 1 protein (WT1, a transcription factor overexpressed in malignant mesothelioma, leukemias, and other solid tumors); CO17-1A (found, e.g., in colon cancer), cancer/testis (CT) antigens such as NY-ESO-1 and LAGE-1, human telomerase reverse transcriptase (hTERT), CD19 or CD20.

A vaccine for an infectious disease may comprise, e.g., a pathogen or any antigen derived from a pathogen (e.g., inactivated or weakened pathogens, pathogen components such as proteins or peptides, etc. In some embodiments an antigen is a surface protein or polysaccharide of, e.g., a viral capsid, envelope, or coat, or bacterial, fungal, protozoal, or parasite cell. In some embodiments an antigen is a toxin, e.g., a toxin produced by a bacterium. A toxin may be provided in an inactivated form, e.g., as a toxoid. An antigen or epitope may be modified, e.g., by chemical treatment (e.g., formaldehyde) or physical treatment (e.g., heat) and/or by conjugation with a second agent. It will be understood that an antigen, e.g., a protein, “derived from” a particular microbe or parasite can be produced using any suitable method, e.g., using recombinant DNA technology in yeast, bacteria, or cell cultures, by chemical synthesis, etc. In some embodiments a variant antigen may be used. For example, a native sequence may be modified to render it more immunogenic. In some embodiments an antigen or epitope is sufficiently similar to a naturally occurring antigen or epitope such that it binds with at least about 10%, 20%, 30%, least 50%, 60%, 70%, 80%, 90%, 95%, or the same affinity to an antigen receptor or antibody that binds to the naturally occurring antigen or epitope. In some embodiments an antigen or epitope is sufficiently similar to a naturally occurring antigen or epitope to elicit a desired response. In various embodiments an antigen can originate from any component of the parasite or can be derived from parasites at any stage of their life cycle of the parasite, e.g., any stage that occurs within an infected organism such as a mammalian or avian organism. In some embodiments an antigen is derived from eggs of the parasite, cysts, or substances secreted by the parasite.

Cell-based immunotherapy may comprise administration of immune system cells, e.g., dendritic cells, CD4+ effector T cells, CD8+ effector T cells, natural killer T cells (which are usually CD8+ T cells), natural killer cells, Tregs, or other immune cells, which may in some embodiments be expanded or activated in vitro prior to administration, for purposes of treating a disease. In certain embodiments a C5L2 modulator may be used as a component of or in combination with cell-based immunotherapy. In some embodiments cell-based immunotherapy comprises dendritic cells, CD4+ effector T cells, CD8+ effector T cells, natural killer T cells, natural killer cells, for purposes of treating an infection or cancer. In some embodiments cell-based immunotherapy comprises Tregs, e.g., for purposes of treating an autoimmune disease or inflammatory disease or inducing tolerance, e.g., to an environmental allergen such as a pollen, dust component, or food allergen.

In certain embodiments a C5L2 activator may be used as a component of or in combination with cell-based immunotherapy comprising administration of dendritic cells, CD4+ effector T cells, CD8+ effector T cells, killer T cells, and/or natural killer cells. Such therapy may be useful, for example, in treating cancer, an infectious disease, or any condition in which an enhanced immune response is desired.

In certain embodiments a C5L2 inhibitor may be used as a component of or in combination with cell-based immunotherapy comprising administration of Tregs, e.g., nTregs. Such therapy may be useful, for example, in treating autoimmune diseases or inflammatory diseases or inducing tolerance, e.g., to an environmental allergen such as a pollen, dust component, or food allergen.

Suitable preparations, e.g., substantially pure preparations of a C5L2 modulator may be combined with pharmaceutically acceptable carriers or vehicles, etc., to produce an appropriate pharmaceutical composition. The term “pharmaceutically acceptable carrier or vehicle” refers to a non-toxic carrier or vehicle that does not destroy the pharmacological activity of the compound with which it is formulated. One of skill in the art will understand that a carrier or vehicle is “non-toxic” if it is compatible with administration to a subject in an amount appropriate to deliver the compound without causing undue toxicity. Pharmaceutically acceptable carriers or vehicles that may be used include, but are not limited to, water, physiological saline, Ringer's solution, sodium acetate or potassium acetate solution, 5% dextrose, and the like. The composition may include other components as appropriate for the formulation desired, e.g., as discussed herein. Supplementary active compounds, e.g., compounds independently useful for treating a subject suffering from a disorder, can also be incorporated into the compositions. The invention provides such pharmaceutical compositions comprising a C5L2 modulator and, optionally, a second active agent useful for treating a subject suffering from a disorder of interest herein.

In some embodiments, the invention provides a pharmaceutically acceptable C5L2 modulator or pharmaceutically acceptable composition comprising a C5L2 modulator packaged together with a package insert (label) approved by a government agency responsible for regulating pharmaceutical agents, e.g., the U.S. Food & Drug Administration or European Medicines Agency. In some embodiments, the invention provides a pharmaceutical pack comprising: (a) a pharmaceutically acceptable C5L2 modulator in concentrated or solid form (e.g., as a lyophilized powder); (b) a pharmaceutically acceptable carrier, diluent, or vehicle. In some embodiments, a suitable carrier, diluent, or vehicle may be provided separately or acquired by a health care provider from an appropriate source. Optionally a pack contains instructions for dissolving or diluting the C5L2 modulator in the carrier, diluent, or vehicle to produce a composition for administration. In some embodiments a package insert states one or more indications that include one or more disorders, e.g., one or more chronic respiratory disorders, of interest herein. In some embodiments, the package insert states particular patient and/or disease characteristics or criteria that define a patient population or disease category for treatment of which the composition has been approved for use. In some embodiments, the package insert specifies that the composition may be or should be administered according to a particular dosing schedule and/or using a particular dosing interval and/or based on evaluating one or more biomarkers.

In general, a pharmaceutical composition can be administered to a subject by any suitable route of administration including, but not limited to, intravascular (intravenous), intramuscular, subcutaneously, by the respiratory route, orally etc. In some embodiments, local administration to a tissue or organ affected by a disorder is used. “Local administration” encompasses (1) administration directly into or near a target tissue or organ, (2) into or near a blood vessel that directly supplies a target tissue or organ, or (3) into a fluid-filled extravascular compartment in or in fluid communication with the target tissue or organ (e.g., inhalational administration where the target tissue or organ is a component of respiratory system such as the lung, intrathecal or intraventricular administration where the target organ or tissue is a component of the central nervous system such as the brain, intrasynovial injection where the target organ or tissue is a joint or synovial membrane). “Near” in this context refers to locations up to 1 cm, 5 cm, or 10 cm from an edge or border of the target tissue, organ, or blood vessel.

It will be understood that “treatment” or “administration” encompasses directly administering an agent or composition to a subject, instructing a third party to administer an agent or composition to a subject, prescribing or suggesting an agent or composition to a subject (e.g., for self-administration), self-administration, and, as appropriate, other means of making an agent or composition available to a subject. If administration is accomplished using an implanted reservoir, administration can refer to causing release of a composition or compound from the reservoir.

Pharmaceutical compositions suitable for injectable use (e.g., intravenous administration, subcutaneous or intramuscular administration) typically include sterile aqueous solutions (where water soluble) or dispersions and sterile powders for the extemporaneous preparation of sterile injectable solutions or dispersion. Sterile solutions can be prepared by incorporating the compound in the required amount in an appropriate solvent, optionally with one or a combination of ingredients such as buffers such as acetates, citrates, lactates or phosphates; agents for the adjustment of tonicity such as sodium chloride or dextrose; antibacterial agents such as benzyl alcohol or methyl parabens; antioxidants such as ascorbic acid, glutathione, or sodium bisulfite; chelating agents such as ethylenediaminetetraacetic acid; and other suitable ingredients etc., as desired, followed by filter-based sterilization. One of skill in the art will be aware of numerous physiologically acceptable compounds that may be included in a pharmaceutical composition. Other useful compounds include, for example, carbohydrates, such as glucose, sucrose, lactose; dextrans; amino acids such as glycine; polyols such as mannitol. These compounds may, for example, serve as bulking agents and/or stabilizers, e.g., in a powder and/or when part of the manufacture or storage process involves lyophilization. Surfactant(s) such as Tween-80, Pluronic-F108/F68, deoxycholic acid, phosphatidylcholine, etc., may be included in a composition, e.g., to increase solubility or to provide microemulsion to deliver hydrophobic drugs. pH can be adjusted with acids or bases, such as hydrochloric acid or sodium hydroxide, if desired. Parenteral preparations may be enclosed in ampoules, disposable syringes or infusion bags or multiple dose vials made of glass or plastic. Preferably solutions for injection are sterile and acceptably free of endotoxin.

Generally, dispersions may be prepared by incorporating the active compound into a sterile vehicle which contains a basic dispersion medium and appropriate other ingredients from those enumerated above. In the case of sterile powders for the preparation of sterile injectable solutions, methods of preparation can include vacuum drying and freeze-drying which yields a powder of the active ingredient plus any additional desired ingredient, e.g., from a previously sterile-filtered solution thereof.

For administration by the respiratory route (inhalation), a C5L2 modulator may be delivered in the form of an aerosol spray from a pressured container or dispenser which contains a suitable propellant. A metered dose inhaler (MDI), dry powder inhaler, or nebulizer may be used. The aerosol may comprise liquid and/or dry particles (e.g., dry powders, large porous particles, etc.). Suitable aqueous vehicles useful in various embodiments nclude water or saline, optionally including an alcohol. In some embodiments the composition comprises a surfactant suitable for introduction into the lung. Other excipients suitable for pulmonary administration can be used. A variety of different devices are available for respiratory administration such as nebulizers, metered dose inhalers (MDI), dry powder inhalers (DPI).

Oral administration may be used in certain embodiments. Oral compositions generally include an inert diluent or an edible carrier. For the purpose of oral therapeutic administration, the active compound can be incorporated with excipients and used in the form of tablets, troches, or capsules, e.g., gelatin capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. A liquid composition can also be administered orally. Formulations for oral delivery may incorporate agents to improve stability within the gastrointestinal tract and/or to enhance absorption.

For topical application, a C5L2 modulator may be formulated in a suitable ointment containing the active component suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated as a suitable lotion or cream containing a compstatin analog suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol, and water.

Systemic administration can also be by transmucosal or transdermal means. For transmucosal or transdermal administration, penetrants appropriate to the barrier to be permeated may be used in the formulation. Such penetrants are generally known in the art, and include, for example, for transmucosal administration, detergents, bile salts, and fusidic acid derivatives. Transmucosal administration can be accomplished, e.g., through the use of nasal sprays or suppositories. In some embodiments, intranasal administration is used. For transdermal administration, the active compounds are typically formulated into ointments, salves, gels, or creams as generally known in the art. The compounds can also be prepared in the form of suppositories (e.g., with conventional suppository bases such as cocoa butter and other glycerides) or retention enemas for rectal delivery.

Methods of local administration to the eye include, e.g., intraocular administration, e.g., intraocular injection, e.g., intravitreal injection. In some embodiments, administration is by choroidal injection, transscleral injection, eyedrops or eye ointments, transretinal, subconjunctival bulbar, intravitreal injection, suprachoroidal injection, subtenon injection, scleral pocket or scleral cutdown injection.

In certain embodiments of the invention, a C5L2 modulator is prepared with carrier(s) that will protect the compound against rapid elimination from the body, such as a controlled release formulation, including implants and microencapsulated delivery systems. For example, a compound may be incorporated into or encapsulated in a microparticle or nanoparticle formulation. Biodegradable, biocompatible polymers can be used, such as ethylene vinyl acetate, polyanhydrides, polyglycolic acid, collagen, polyorthoesters, polyethers, polylactic acid, PLGA, etc. Liposomes or other lipid-based particles can be used as pharmaceutically acceptable carriers. These can be prepared according to methods known to those skilled in the art, for example, as described in U.S. Pat. No. 4,522,811 and/or other references listed herein. Depot formulations may be used from which C5L2 modulator is released from the depot over time. One of ordinary skill in the art will appreciate that the materials and methods selected for preparation of a controlled release formulation, implant, etc., should be such as to retain activity of the compound.

In some embodiments, a C5L2 modulator is provided or used in combination with one or more additional active agent(s) useful to treat a disorder of interest herein (see, e.g., Brunton, L L, et al. (eds.), Goodman and Gilman's The Pharmacological Basis of Therapeutics, (e.g., 11th or 12th edition), McGraw-Hill, for examples of such agents.) In some embodiments one or more additional active agents is administered in the same composition as a C5L2 modulator. In some embodiments one or more additional active agents is administered in a separate composition, which separate composition may be administered prior to, at approximately the same time as, or after administeration of a C5L2 modulator. In some embodiments, use of a C5L2 modulator allows reduction in dose and/or frequency of administration of an additional active agent while maintaining at least equivalent disease control and/or benefit to the subject. It will be understood that pharmaceutical compositions comprising an additional active agent may be prepared using pharmaceutically acceptable carriers and/or preparation methods described herein or known in the art, and administered using routes of administration described herein or known in the art. In some embodiments an additoinal active agent comprises a cytokine.

When two or more therapies (e.g., compounds or compositions) are used or administered “in combination” with each other, they may be given at the same time, within overlapping time periods, or sequentially (e.g., separated by up to 2-4 weeks in time), in various embodiments of the invention. They may be administered via the same route or different routes in various embodiments. They may be administered in either order in various embodiments. In some embodiments, the compounds or compositions are administered within 4, 8, 12, 24, 48, 72, or 96 hours of each other. In some embodiments, a first agent is administered prior to or after administration of the second agent, e.g., sufficiently close in time that the two agents are present at useful levels within the body at least once. In some embodiments, the agents are administered sufficiently close together in time such that no more than 90% of the earlier administered composition has been metabolized to inactive metabolites or eliminated, e.g., excreted, from the body, at the time the second compound or composition is administered. In some embodiments, the agents are administered sufficiently close together in time such that no more than 2 weeks has elapsed since the earlier administered agent has been metabolized to inactive metabolites or eliminated, e.g., excreted, from the body, at the time the second agent is administered.

It will be appreciated that a C5L2 modulator and/or additional active agent(s) can be provided as a pharmaceutically acceptable salt. Pharmaceutically acceptable salts include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Also, pharmaceutically-acceptable salts can be prepared as alkaline metal or alkaline earth salts, such as sodium, potassium or calcium salts, if appropriate depending on the identity of the active agent.

It will be understood that the pharmaceutically acceptable carriers, compounds, and preparation methods mentioned herein are exemplary and non-limiting. See, e.g., Remington: The Science and Practice of Pharmacy. 21st Edition. Philadelphia, Pa. Lippincott Williams & Wilkins, 2005, for additional discussion of pharmaceutically acceptable compounds and methods of preparing pharmaceutical compositions of various types.

A compound or composition, e.g., a pharmaceutical composition, can be used or administered to a subject in an effective amount. In some embodiments, an “effective amount” of an active agent, e.g., a C5L2 modulator, (or composition containing an active agent) refers to an amount of the active agent (or composition) sufficient to elicit one or more biological response(s) of interest in, for example, a subject to whom the active agent (or composition) is administered. As will be appreciated by those of ordinary skill in the art, the absolute amount of a particular agent that is effective may vary depending on such factors as the biological endpoint, the particular active agent, the target tissue, etc. Those of ordinary skill in the art will further understand that an “effective amount” may be administered in a single dose, or may be achieved by administration of multiple doses. For example, in some embodiments, an effective amount may be an amount sufficient to achieve one or more of the following: (i) inhibit or reduce the severity of one or more symptoms of the disease (e.g., pain); (ii) inhibit or reduce the severity of one or more signs of the disease; (iii) improve functional activity of at least one tissue or organ affected by the disease; (iv) reduce need for concomitant medications; (v) inhibit or prevent a long-term pathological change associated with the disorder; (vi) improve quality of life and/or overall daily functioning; (vii) reduce average number and/or length of emergency room visits and/or hospitalizations; (viii) reduce mortality; or (ix) any combination of the foregoing.

Indicators of inflammation include, e.g., the presence of increased numbers of inflammation-associated cells such as white blood cells (e.g., neutrophils, eosinophils, mast cells, lymphocytes, macrophages) and/or inflammatory mediators (e.g., chemokines (e.g., eotaxin, thymus and activation-regulated chemokine (TARC), macrophage-derived chemokine (MDC)), pro-inflammatory cytokines or other mediators (e.g., histamine, cysteinyl leukotrienes, nitric oxide) in the blood and/or in the relevant tissue, as compared with a suitable reference level, e.g., a normal level. For example, the number and/or concentration of cells and/or mediators in a subject suffering from an inflammatory disease may be above the upper limit of the normal range in subjects not suffering from a disorder or may be greater than a value (or average value) measured in that subject when the subject's disorder is well controlled. A reduction (e.g., in symptom severity and/or frequency) can be statistically significant and/or clinically meaningful within the sound judgment of a physician or other medical practitioner. Determining whether a disorder is effectively treated is within the sound judgment of a physician or other medical practitioner. Art-accepted guidelines may be used.

In some embodiments an effective amount results in reduction of at least one parameter associated with Th1 cells and/or Th1 activity. In some embodiments an effective amount reduces the level of at least one cytokine associated with Th1 cells and/or Th1 activity, e.g., a cytokine that promotes Th1 cell generation and/or activity or a cytokine produced by Th1 cells, e.g., IFN-γ.

In some embodiments an effective amount results in reduction of at least one parameter associated with Th17 cells and/or Th17 activity. In some embodiments an effective amount reduces the level of at least one cytokine associated with Th17 cells and/or Th17 activity, e.g., a cytokine that promotes Th17 cell formation and/or activity or a cytokine produced by Th17 cells, e.g., IL-17, IL-21, IL-22, or IL-23.

In some embodiments an effective amount results in reduction of IL-6 level, e.g., in the blood and/or in a tissue or organ affected by an IL-6 mediated disease or in extracellular fluid present in such tissue or organ. In some embodiments an effective amount results in reduction of IL-1β level, e.g., in the blood and/or in a tissue or organ affected by an IL-1β mediated disease or in extracellular fluid present in such tissue or organ.

One of skill in the art will be aware of appropriate methods to assess the afore-mentioned effects and other effects of interest. Symptoms can be assessed using standardized instruments (e.g., questionnaires) known in the art. Any of a variety of different health-related quality of life (HRQOL) instruments can be used, which can be generic or specifically associated with a particular disease or body system. Functional tests may be used to assess functional activity of tissue(s) or organ(s) affected by a disease. In certain embodiments efficacy in treating an infectious disease may be assessed at least in part by detecting a reduction or absence of the particular pathogen, e.g., in a blood or tissue sample. In certain embodiments, objective response of a subject with cancer e.g., as defined using the Response Evaluation Criteria In Solid Tumors (RECIST) guideline (Therasse, P., et al., Journal of the National Cancer Institute, 92(3): 205-216 (2000) or revised RECIST guideline (version 1.1) (Eisenhauer, E. A., et al., Eur J Cancer. 45(2):228-47 (2009)) or other accepted guidelines, e.g., for hematological malignancies or brain tumors, may be used. For example, an outcome may be classified as a complete response, partial response, progressive disease, or stable disease.

Inflammation-associated cells and/or mediators may be assessed, for example, in a suitable sample such as blood, plasma, induced sputum, BAL fluid, synovial fluid, cerebrospinal fluid, and/or a tissue sample (e.g., obtained from a biopsy of the relevant tissue). Cells, e.g., inflammation-associated cells can be detected and optionally quantified using, e.g., electron microscopy, optical microscopy (optionally using suitable chemical stains or antibodies to particular markers (immunohistochemistry), flow cytometry, or other suitable methods. Mediator (e.g., cytokine) levels may be measured using, e.g., antibody-based assays such as ELISA assays, bead array assays (such as the Luminex xMAP technology or Cytometric Bead Array (CBA) system from BD Biosciences), antibody array assays, or appropriate bioassays. Expression of mediators can alternately or additionally be assessed by measuring the level of mRNA encoding such mediators (e.g., using any suitable method for measuring RNA level such as reverse transcription PCR, hybridization to oligonucleotide or cDNA arrays, RNA-Seq (e.g., methods making use of high-throughput sequencing technologies to sequence cDNA to obtain information about RNA in a sample), etc.).

In general, a control subject can be, e.g., an untreated subject or a subject treated with a placebo. An “untreated subject” may be a subject who has not received treatment with an agent intended for treatment of the particular disorder in question within the preceding 1, 2, 3, 4, 5, or 6 months. Historical control information can be used. In some embodiments, a subject can serve as his or her own control. For example, one or more parameters can be measured once or more prior to treatment and once or more during and/or following treatment. In some embodiments, an “active control” (or “active comparator”) is used, wherein a biological effect of a C5L2 modulator is compared with that of a compound known to affect the parameter being assessed. For example, a compound that is approved for use for treating a particular disorder may be used. It will be appreciated that if an active comparator is used as a control, an effective amount of a C5L2 modulator may have less, more, or about the same effect as the active comparator at one or more time points in various embodiments.

In some embodiments, a non-human animal model is used, for example, to help guide selection of a dose, dose range, or formulation for testing in human, to assess one or more biological effect(s), etc. In some embodiments a non-human animal is a monkey (e.g., cynomolgus monkey; Macaca fascicularis), rodent (e.g., mouse, rat, hamster), sheep, guinea pig, min-pig, etc.

In general, appropriate doses of C5L2 modulator or other active agent depend at least in part upon the potency of the agent, route of administration, etc. In general, dose ranges that are effective and well tolerated can be selected by one of ordinary skill in the art. Such doses can be determined using clinical trials as known in the art. Optionally, a dose may be be tailored to the particular recipient, for example, through administration of increasing doses until a preselected desired response is achieved. If desired, the specific dose level for any particular subject may be selected based at least in part upon a variety of factors including the activity of the specific compound employed, the particular condition being treated and/or its severity, the age, body weight, general health, route of administration, any concurrent medication, and/or the degree of aberrant cytokine or T cel level or activity measured in one or more samples obtained from the subject. In some embodiments an effective amount or dose ranges from about 0.001 to 500 mg/kg body weight, e.g., about 0.01 to 100 mg/kg body weight, e.g., about 0.1 to 50 mg/kg body about 0.1 to 20 mg/kg body weight, e.g., about 1 to 10 mg/kg.

### EXAMPLES

Materials and Methods

Human CD4⁺ T Cell and Monocyte Isolation:

Human blood was obtained from healthy volunteers following ethical guidelines set by Kings College London (KCL) ethics committee (REC: 09/H0804/72). PBMCs were isolated by density centrifugation (Ficoll-Paque PLUS; GE healthcare, Uppsala, Sweden). Cell subsets were isolated by magnetic separation following manufacturer's instructions (Miltenyi Biotec, Auburn, Calif.). Monocytes were positively selected using anti-CD14 microbeads (average purity 97%) and CD4⁺ T cells were isolated from the CD14-negative fraction using anti-CD4 microbeads (average purity 98%). Alternatively, CD4⁺ T cells were isolated from purified PBMCs by cell sorting after staining with antibodies to CD14 and CD4 and CD3.

CD4⁺ T Cell and Monocyte Activation:

CD4+ T cells and CD14+ monocytes were cultured in medium (RPMI-1640 medium (Sigma-Alich, Suffolk, UK), supplemented with 1% GlutaMAX, 1% penicillin/streptomycin (both Life Technologies, Paisley, UK) and 10% FBS (HyClone Laboratories, Logan, Utah). In the case of T cells media was supplemented with 25 U/ml IL-2 (Sigma-Aldrich; or with concentrations indicated in figure legends). CD4+ T cells (2.5×10⁵) were cultured alone or together with plate-bound anti-CD3 (OKT3, Biolegend, San Diego, Calif.), anti-CD3 and anti-CD28 (CD28.2, BD Pharmingen, San Jose, Calif.) or anti-CD3 and anti-CD46 (TRA-2-10, provided by John Atkinson, Washington University, St Louis, Mo.) all immobilised onto 48-well plates at concentrations of 2 μg/ml PBS overnight at 4° C. Monocytes (2.5×10⁵) were cultured either alone or in the presence of LPS or Flagellin (1 μg/ml). In certain experiments antagonists for C5aR (PMX53 (10 uM) provided by John Lambris, University of Pennsylvania, PA) or C5aR and C5L2 (Dual receptor antagonist A8delta71-73 (7 μM) a gift from Jorg Kohl, University of Lubeck, Germany), or a carboxypetidase (CP) A and B inhibitor, which also effectively inhibits CPM (Sigma CO279; 25 nM) were added to cultures. Cells were cultured at 37° C. and 5% CO₂ and cytokine secretion assessed at 12, 24 or 36 h post activation.

Assessment of C5 and C5a Expression by Resting and Activated Human CD4⁺ T Cells:

CD4+ T cells were isolated and left either non-activated or were activated with immobilized antibodies. At 20 h post activation, intracellular C5 and C5a expression was determined by FACS using an intracellular staining protocol. The antibody to human C5 was purchased from Serotec (MCA2610) and used in 1:200 dilution. The antibody to C5a was a gift from Jorg Kohl (University of Lubeck), used in a 1:100 concentration and only recognises the cleaved C5a neo-epitope but not the C5a portion contained within the non-processed C5 α-chain.

Assessment of C5aR and C5L2 Expression by Human CD4⁺ T Cells:

Intracellular and extracellular expression of C5aR and C5L2 by resting and activated CD4⁺ T cells was assessed using FACS. Freshly purified T cells or T cells activated for 1 h or 20 h with immobilized antibodies to either CD3, CD3 and CD28 or CD3 and CD46 were stained with mAbs to C5aR (BioLegend, 344303) or C5L2 (BioLegend, 342403) or specific isotype control antibodies with or without fixation and permeabilization (BD Fix/Perm Kit) and assessed for receptor expression by FACS analysis.

Cytokine Measurements:

The Th1/Th2/Th17 Cytometric Bead Array (BD Bioscience) was used to quantify cytokine production by CD4⁺ T cells and monocytes following 12 or 36 h culture under distinct activation conditions. Samples were processed following manufacturer's instructions and acquired using BD FACScan. IL-1β amounts secreted into the cell media were measured by ELISA, according to the manufacturer's protocol (R&D Systems, Abingdon, UK).

nTreg Isolation and Suppression Assay:

nTregs were isolated essentially as described in Yates, J., et al., Int Immunol. (2007) 19(6):785-99. Briefly, CD4+ T cells were purified via negative selection using the Miltenyi kit, stained with anti-CD4, anti-CD25 and anti-CD127, and sorted based on a CD4⁺/CD25^{high}/CD127^{low} expression phenotype

The nTreg suppression assay was carried out as previously described using the CFSE method (Afzali et al., (2013) Eur J Immunol. 43(8):2043-54) with the following modification: Purified nTregs were incubated with 7 μM of the C5aR/C5L2 double antagonist for 8 h. The cells were then washed twice with PBS and used for the assay described below.

Principle

Label target cells (T effectors (Teff) usually) with 1 μM CFSE as described Activate cells with and without Tregs in serial dilution (constant Teff numbers) CFSE will be evenly split among daughter cells, so peaks on flow will correspond to each round of division (halving of CFSE)

The percentage of dividing precursors can be calculated this way.

In the presence of Tregs, the suppression of division can be estimated.

### Reagents

Tregs at 1×10⁶/mL concentration in 10% HS/RPMI
CFSE labelled Teff at 5×10⁵/mL concentration in 10% HS/RPMI
Unlabelled Teffs
96 well U-bottomed plate
Activating antibodies, either anti-CD3/CD28 beads or coated wells

### Protocol

1. Prepare Tregs at 1×10⁶/mL.
2. Add 100 μL of this to row B of a column in 96 well plate (=1×10⁵ cells in total)
3. Add 50 μL of 10% HS/RPMI into rows C to F of the same column of the plate
4. Take 50 μL of the cell solution in row B and serially dilute all the way down the column 1:1 at each step, ending up with 50 μL in row G.
5. Add 50 μL of the Treg solution into row H (=5×10⁴ cells. This will act as the Treg alone condition).
6. To all of these wells, add 100 μL of the Teff solution (=5×10⁴ Teff cells per well), thus making Teff:Treg ratios of 1:1, 2:1, 4:1, 8:1, 16:1 and 32:1
7. In three separate wells without Tregs, add 100 μL of the Teff solution (these will be the 1:0 condition) and in one separate well add 200 μL of the Teff solution (this will be the 2:0 ratio)
8. Add unlabelled Teffs to some empty wells at the same cell number (useful for calibrating the flow cytometer at the end)
9. If adding Dynal/Invitrogen anti-CD3/CD28 beads, add 0.03125 μL per well in 50 μL volume of 10% HS/RPMI. This gives a bead:cell ratio of 0.025 (1 to 40) for this assay. Leave one Teff alone condition unactivated.
10. Adjust all wells to 250 μL volume.
11. Put in incubator for 4 days.
12. Acquire a small number of CFSE labelled Teffs on flow cytometer to ensure correct labelling *.
13. On day 4, remove 110 μL sup from each well and store for cytokine estimation.
14. Acquire FL1 fluorescence of each well on day 4 and calculate suppression of CFSE dilution by Tregs (different methods exist for this)
   * If there is inadequate CFSE labelling, pulse with ³H-Thymidine on day 5 instead, as a readout for suppression.

### Example 1: Expression of C5, C5a, and C5a Receptors by Resting and Activated T Cells

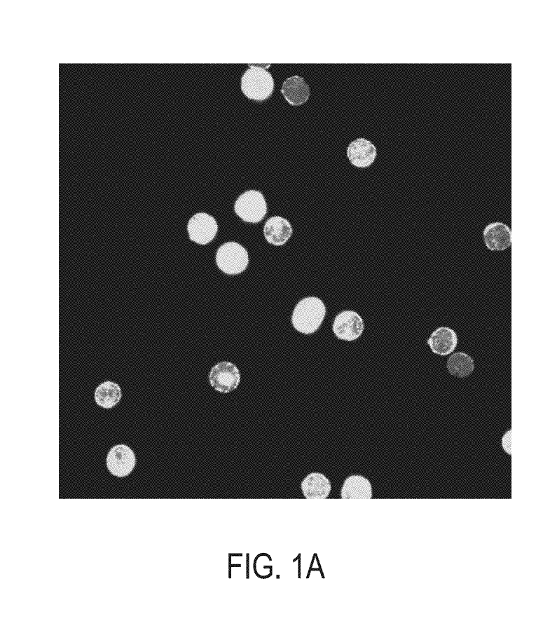

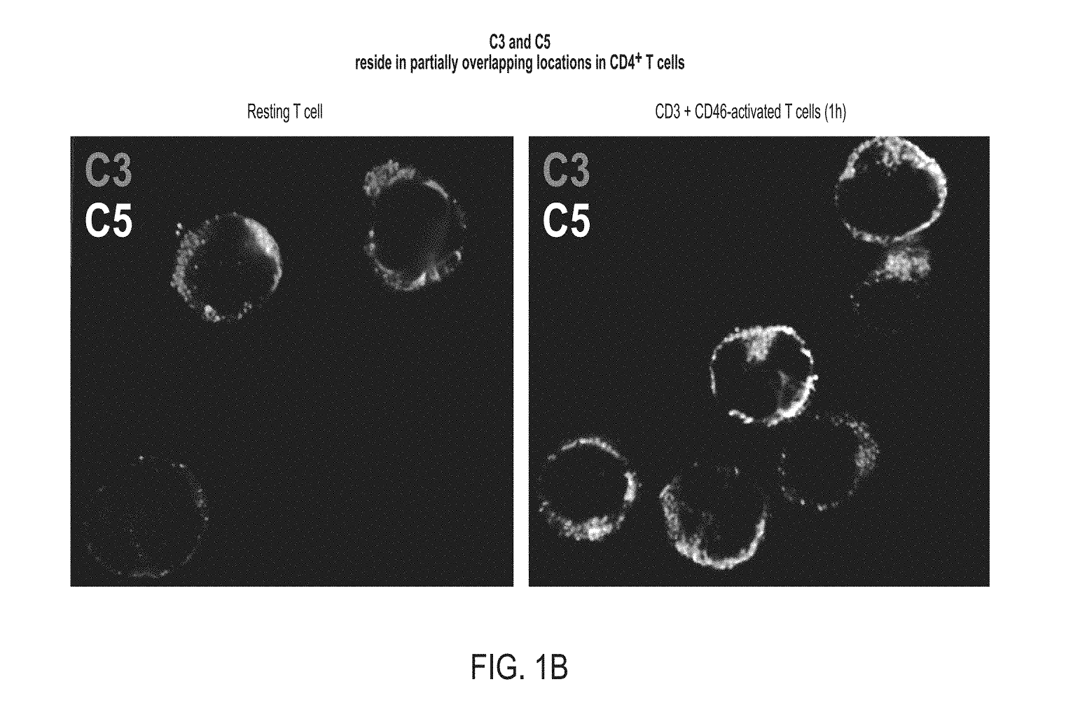

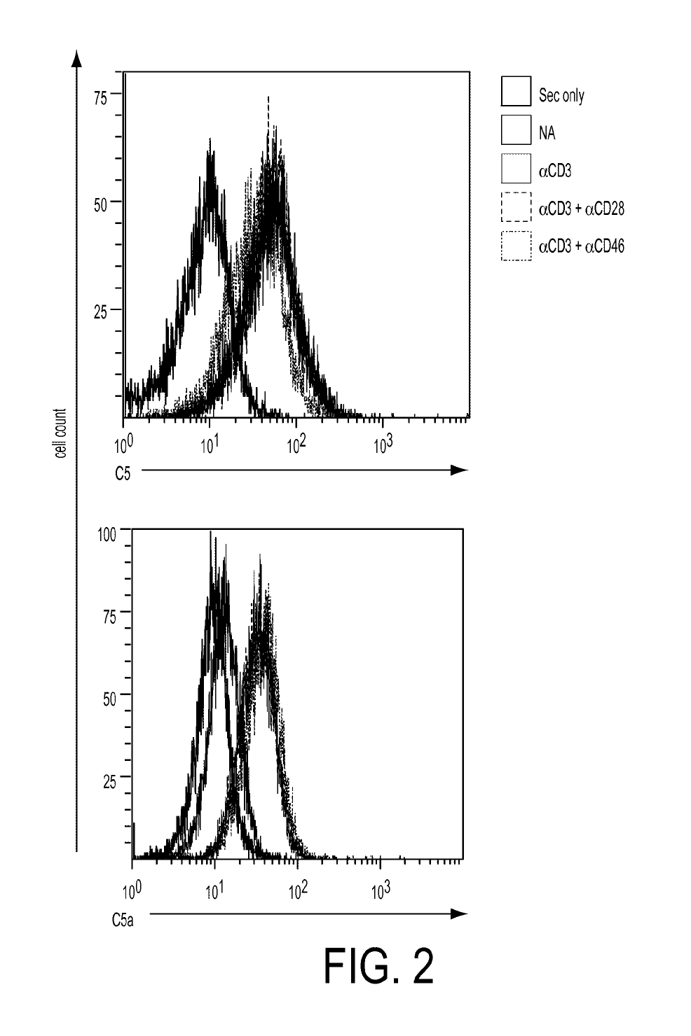

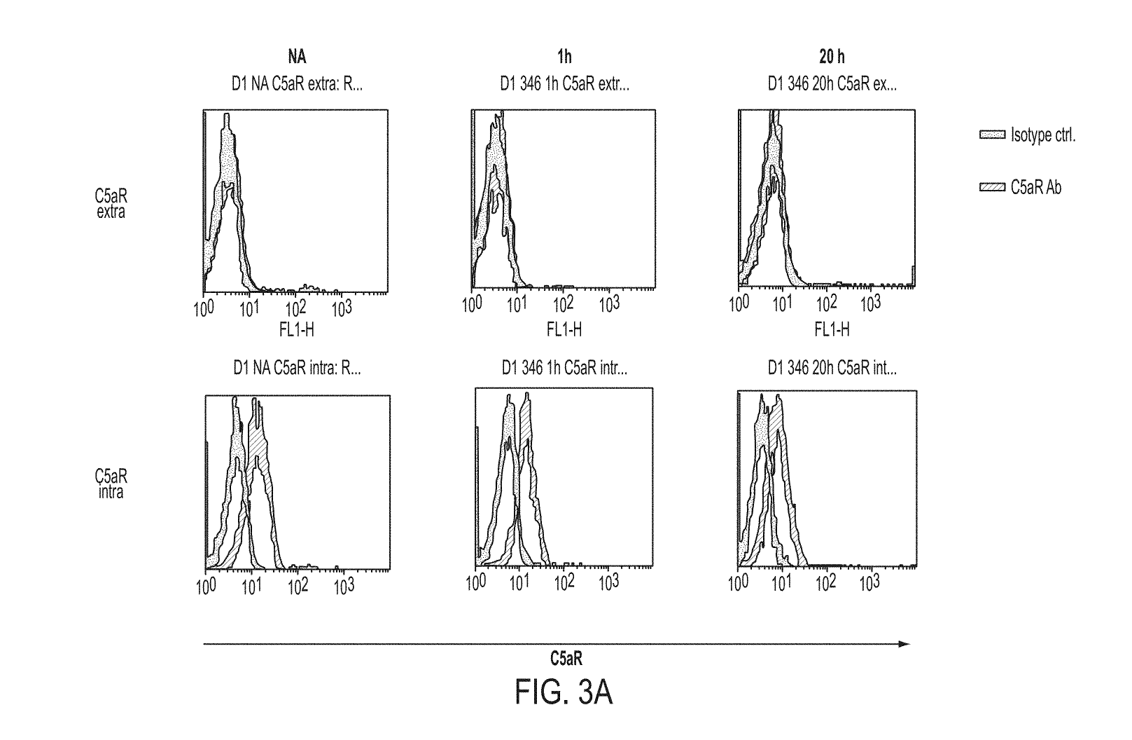

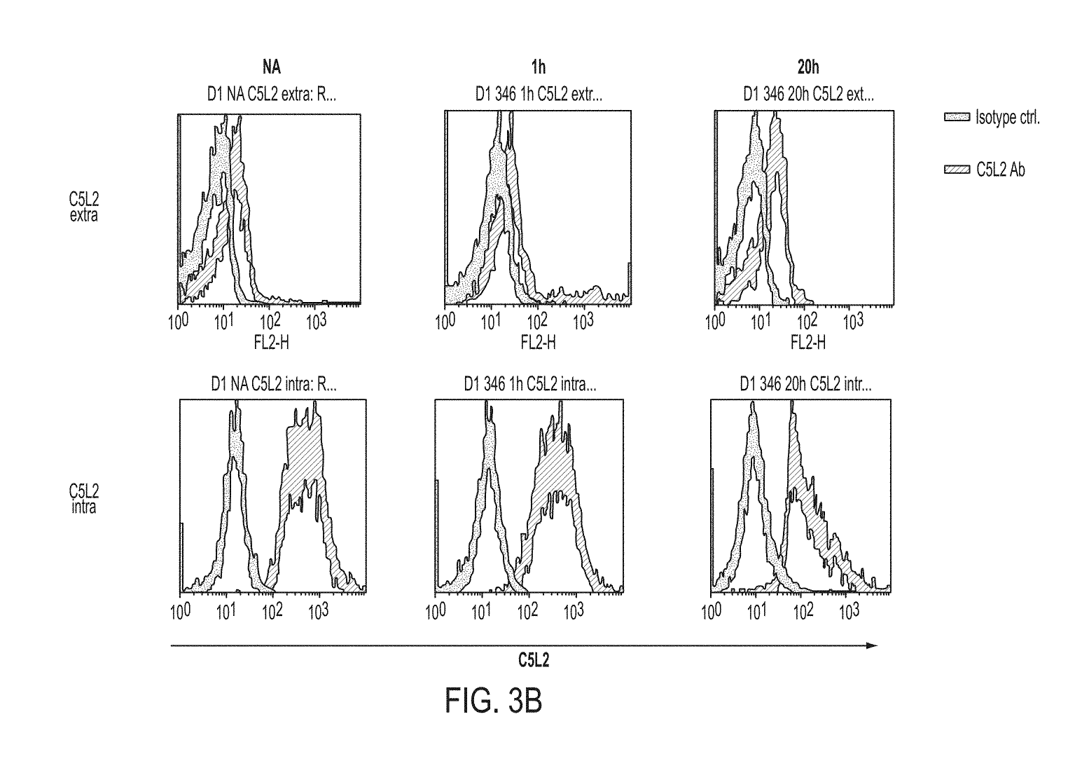

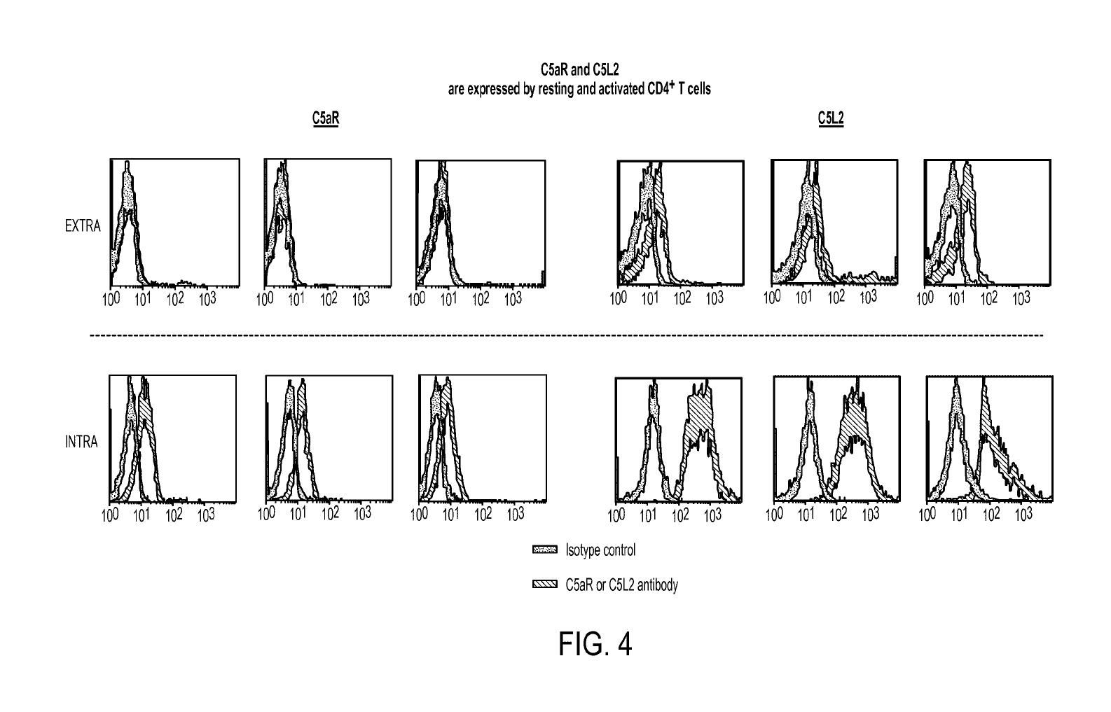

To explore the possible role of the C5 axis in T cell biology, the presence of C5, C5a, C5aR, and C5L2 inside or on the surface of CD4⁺ T cells was assessed using antibody staining and detection using immunofluorescence microscopy or FACS. FIG. 1A shows resting CD4⁺ T cells stained for intracellular C5. Robust levels of C5 are evident. FIG. 1B shows that C3 and C5 reside in partially overlapping locations in CD4+ T cells. FIG. 2 shows FACS data demonstrating staining for intracellular C5 as well as C5a (detected via an antibody that only recognizes the C5a neo-epitope and not the C5a portion still contained within the uncleaved C5 α-chain) in resting and activated CD4⁺ T cells. FIG. 3A shows FACS data demonstrating C5aR expression in resting and activated human CD4⁺ T cells but not on the cell surface. CD3 or CD3+CD28-activation of cells resulted in similar C5aR expression profiles (not shown). FIG. 3B shows C5L2 expression in resting and activated human CD4+ T cells, demonstrating presence of both intracellular and extracellular C5L2. Whereas both receptors were detected intracellularly, only C5L2 was observed on the cell surface. CD3 or CD3+CD28-activation of cells resulted in similar C5L2 expression profiles (not shown). FIG. 4 summarizes the data in FIGS. 3A and 3B. In summary, these data demonstrate expression and intracellular cleavage of C5 by CD4+ T cells.

### Example 2: C5L2 Blockage Causes Increased IFN-γ and IL-17 Secretion by Activated T Cells

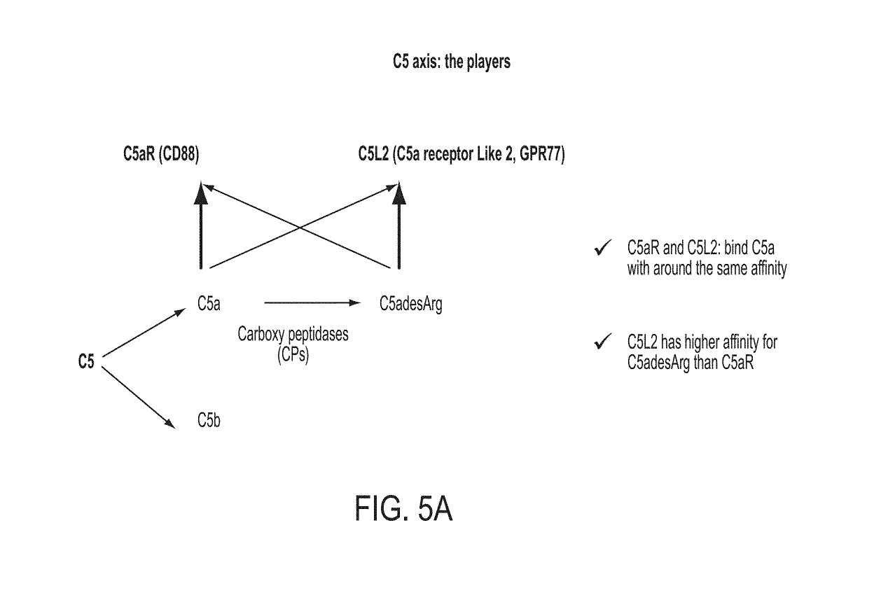

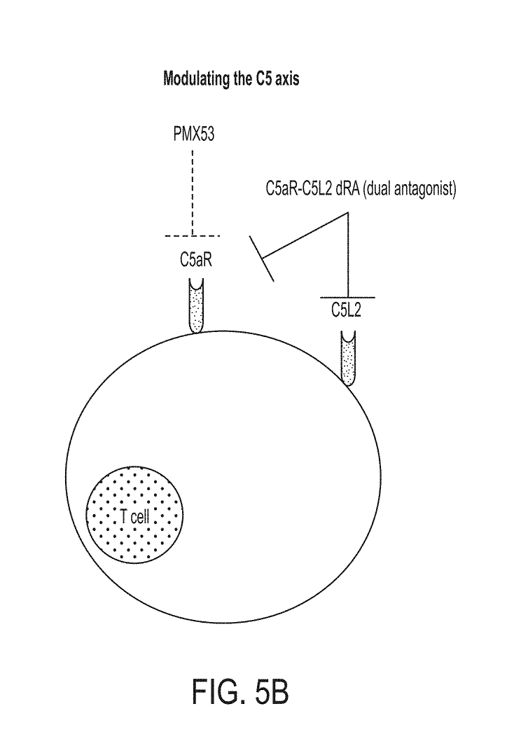

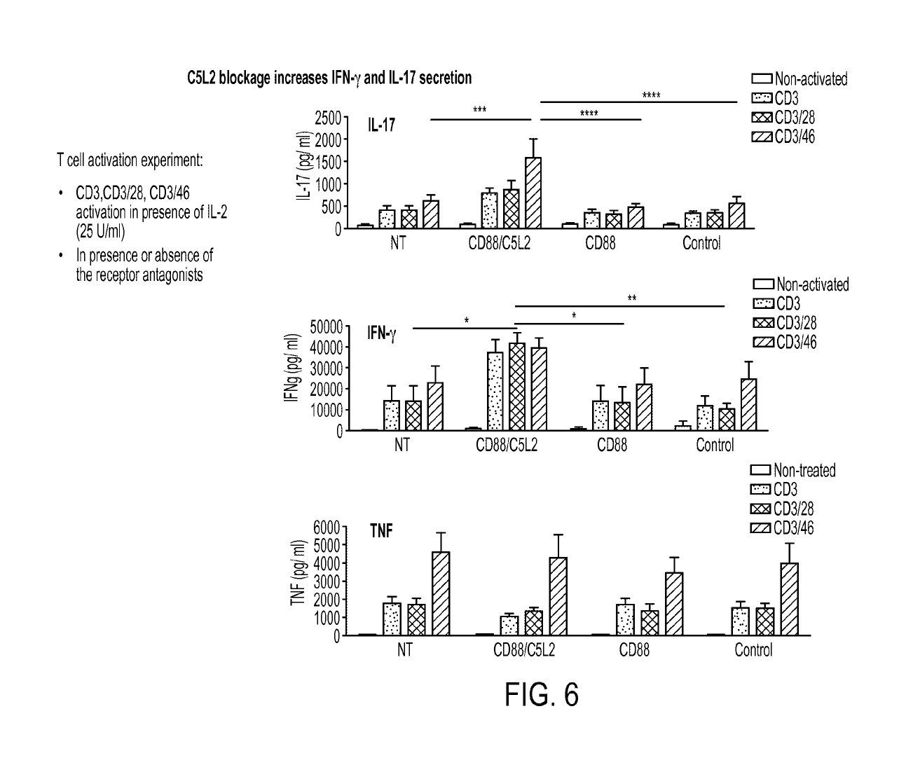

To explore potential functional roles of C5 and/or its cleavage products in T cells, the effect of blockade of either the C5a receptor (C5aR) or both C5aR and the alternative C5a receptor (C5L2) on secretion of various cytokines by T cells was examined. A schematic diagram of the receptor blocking activities of the C5aR antagonist and the dual antagonist is shown in FIG. 5. Exposing cells to these agents allows measurement of the effects of blocking C5L2. If an effect is observed in cells exposed to the dual antagonist but not in cells exposed to the C5aR antagonist, one may conclude that the effect was due to blockade of C5L2. We found that dual C5aR/C5L2 blockade caused increased secretion of IL-17 and IFN-γ by activated CD4+ T cells, whereas C5aR blockage alone had no effect (FIG. 6). Thus, C5L2 blockade was responsible for causing increased secretion of IL-17 and IFN-γ. T cells from a C5-deficient patient (unable to produce C5) presented with deregulated Th1 and Th17 responses, characterized by significantly increased IFN-γ and IL-17 production similar to that observed when CD4+ T cells from normal donors were exposed to the dual antagonist.

### Example 3: C5L2 Blockage Causes Increased IL-6 Secretion by Resting and Activated T Cells

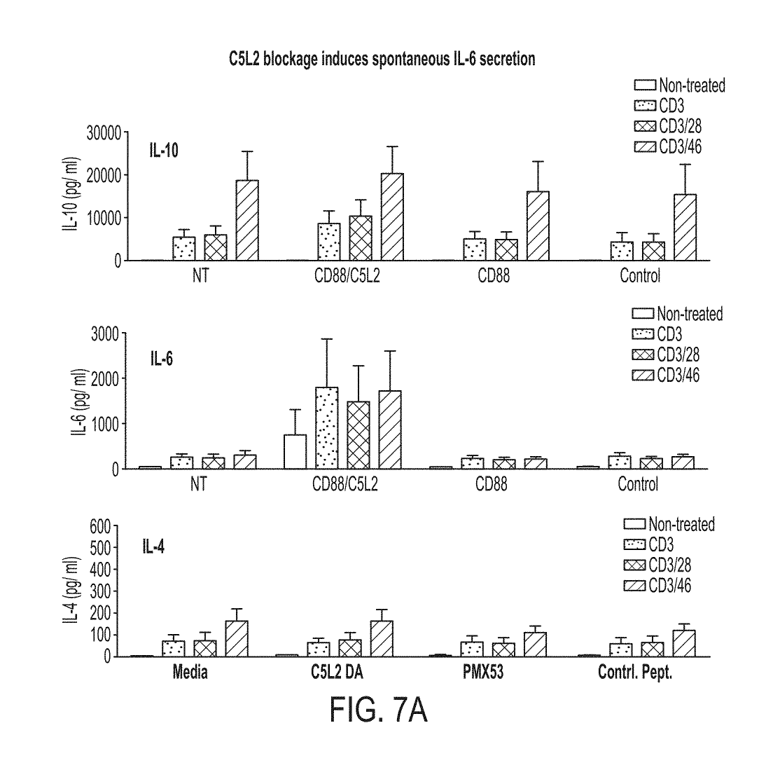

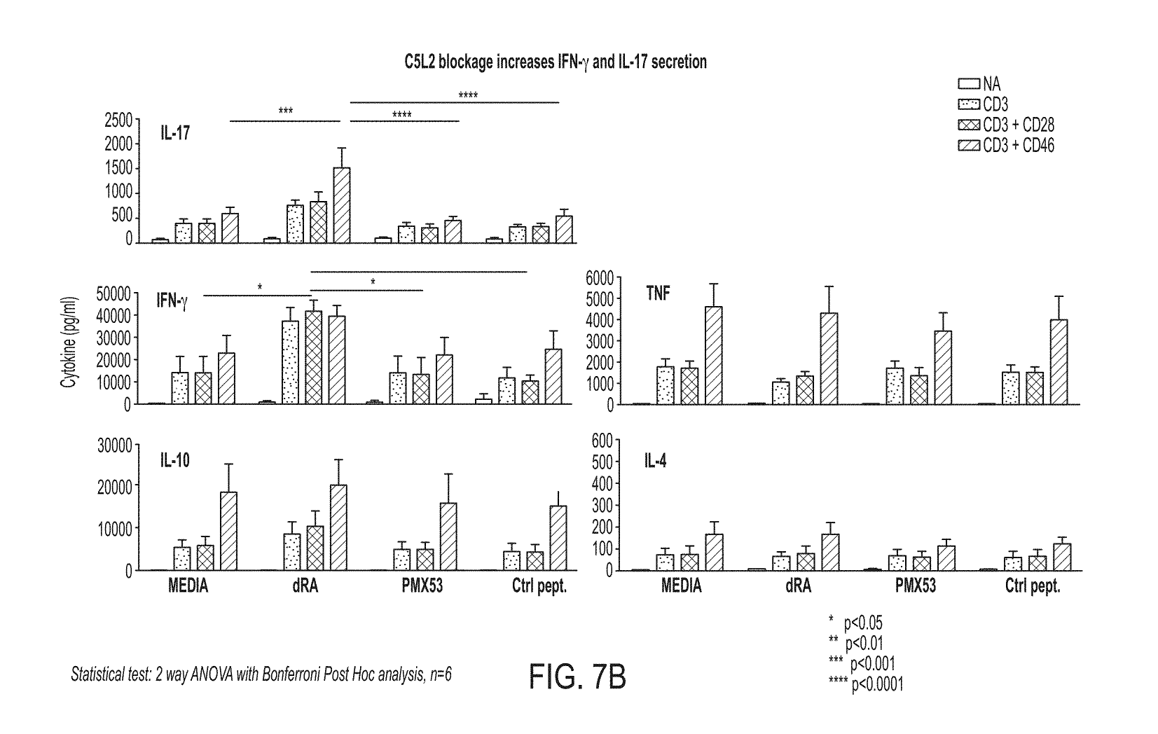

The effects of C5aR blockade and dual C5aR/C5L2 blockade on secretion of additional cytokines was determined as described in Example 2. It was found that dual C5aR/C5L2 blockade caused increased secretion of IL-6 by resting and activated CD4+ T cells, whereas C5aR blockage alone had no effect (FIG. 7A). Thus, C5L2 blockade was responsible for causing increased secretion of IL-6 by these cells.

### Example 4: C5L2 Blockage Causes Increased IL-1β Secretion by Resting and Activated CD4+ T Cells and by Monocytes

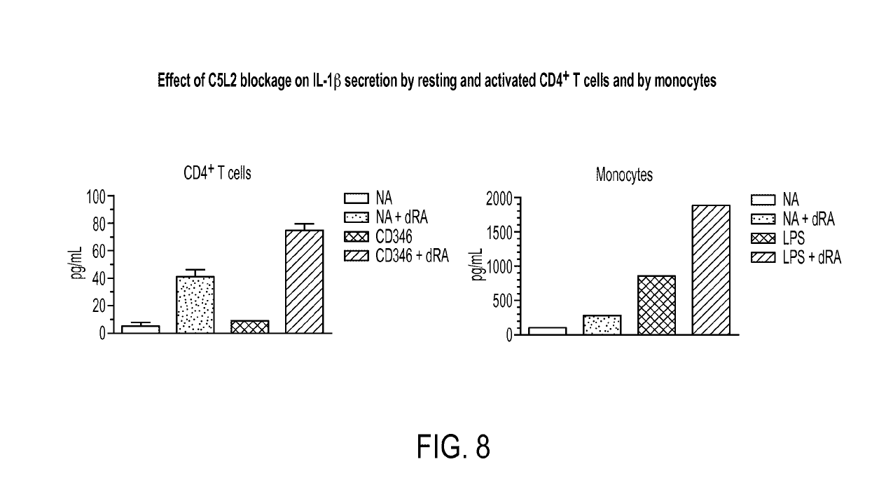

Next the effect of C5L2 blockage on IL-1β secretion by resting and activated CD4+ T cells and by monocytes was assessed. It was found that exposure to the dual C5aR/C5L2 antagonist resulted in increased IL-1β secretion by both resting and activated T cells and also by resting and LPS-activated monocytes (FIG. 8). Although an effect on C5aR cannot be ruled out by this experiment, it appears most likely that the effect of the dual antagonist occurred due to its blockade of C5L2.

### Example 5: C5L2 Blockade Induces Pro-Inflammatory Cytokine Release by Human Monocytes

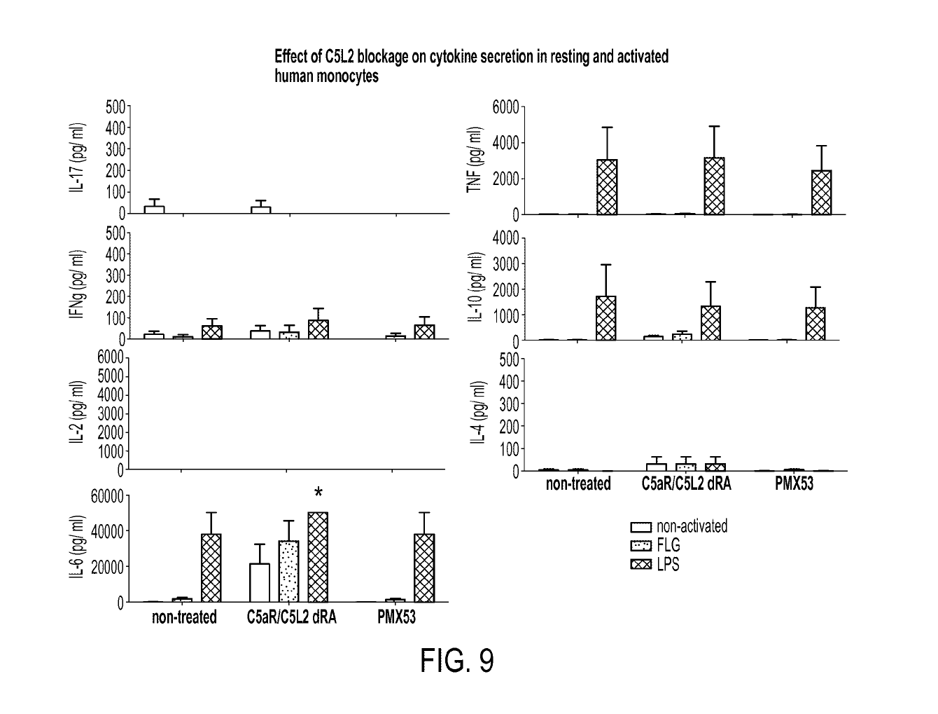

Effects of C5L2 blockade on secretion of additional cytokines by non-activated monocytes as well as monocytes activated with either Flagellin or LPS were assessed. The C5aR/C5L2 double receptor antagonist promoted secretion of IL-6 by both resting and Flagellin-stimulated monocytes (FIG. 9), whereas the C5aR antagonist had no effect.

### Example 6: Carboxypeptidase M (CPM) Inhibition Reduces the Effect of C5L2 Blockade on Cytokine Secretion

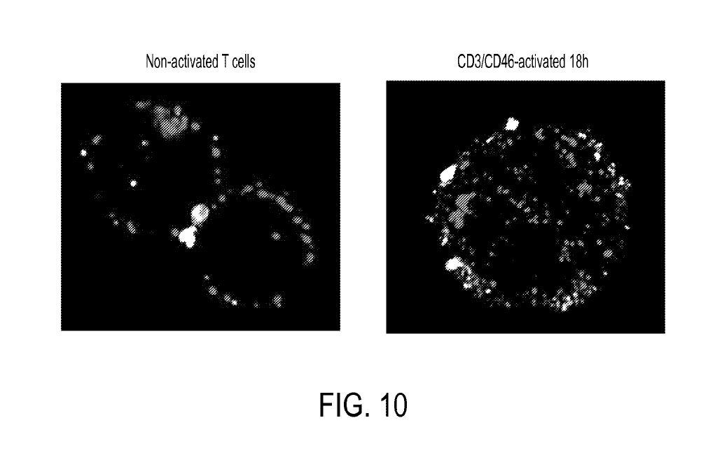

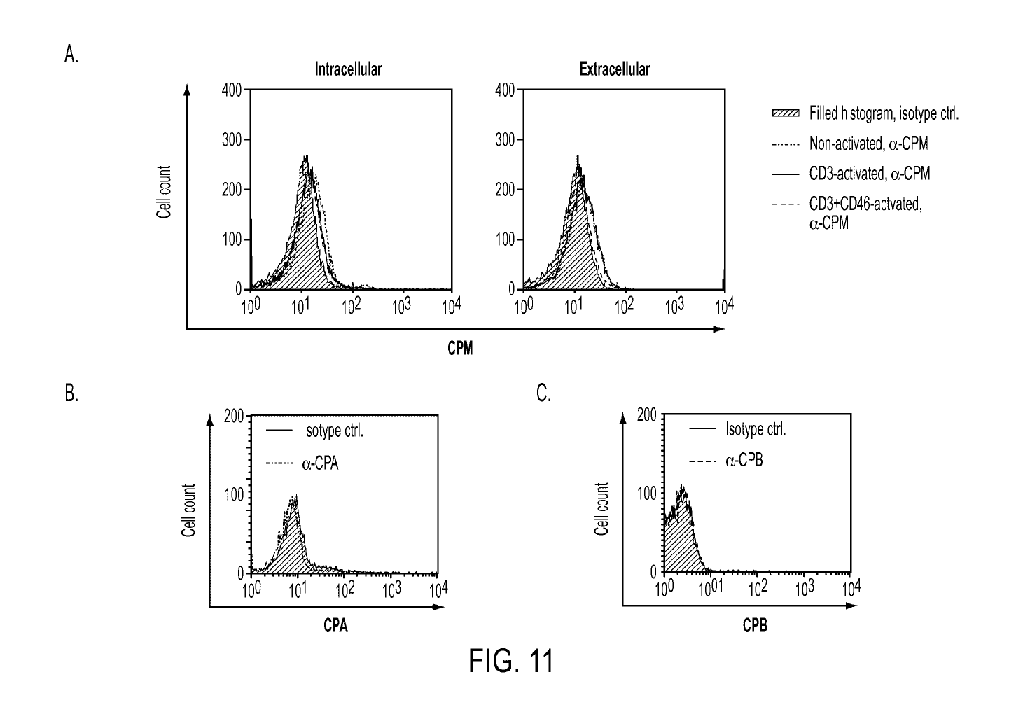

It was hypothesized that signaling by C5L2 may be stimulated by autocrine production of C5adesArg. C5adesArg is generated from C5a by the action of carboxypeptidases. Expression of carboxypeptidase M (CPM), a cell-membrane bound carboxypeptidase capable of cleaving off C-terminal arginine, was assessed and found to be expressed by resting and activated CD4+ T cells, with expression increasing during activation (FIG. 10). Intracellular and cell surface expression of CPM by resting and activated CD4+ T cells was confirmed by FACS (FIG. 11(A)). It was also confirmed that these cells do not express pancreatic carboxypeptidase A or B (FIGS. 11(B) and (C)). The membrane localization and expression pattern of CPM strongly suggested that it is responsible for autocrine production of C5adesArg by T cells and likely monocytes.

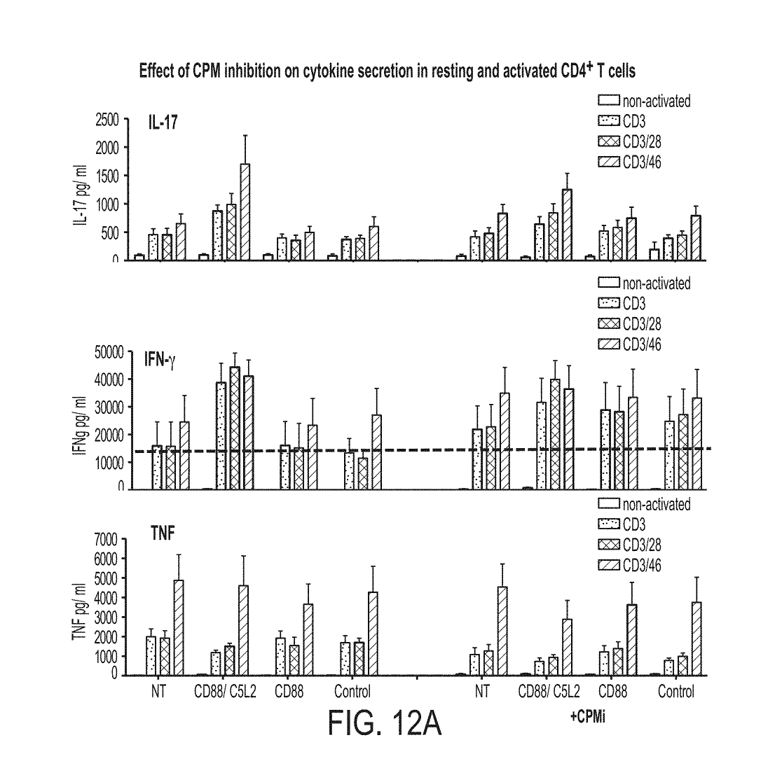

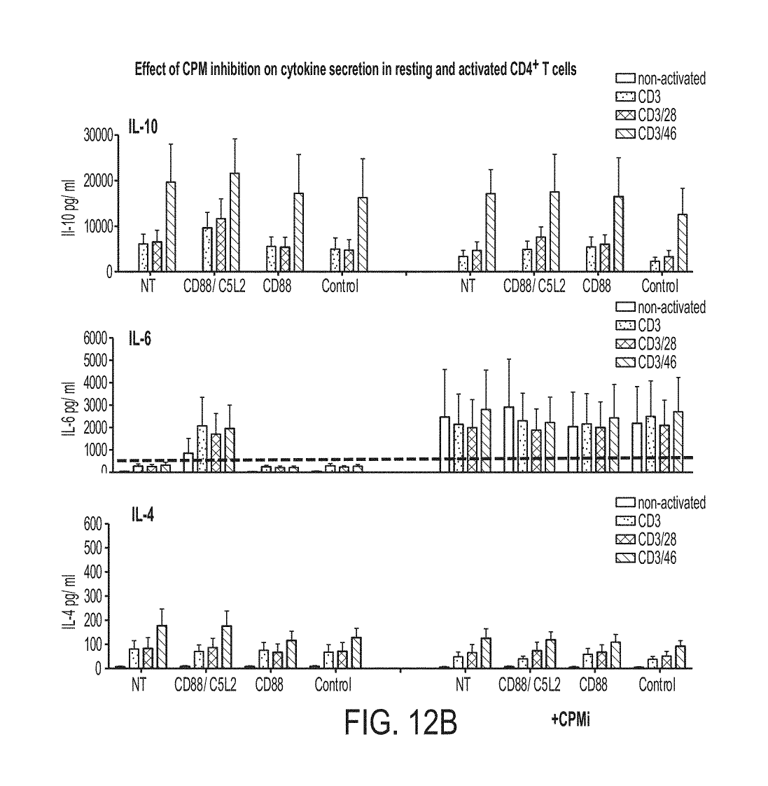

The effect of carboxypeptidase M (CPM) inhibition on cytokine secretion in resting and activated CD4+ T cells was assessed using cytokine bead array (CBA). Cells were treated with either C5aR/C5L2 dual receptor antagonist or C5aR antagonist with or without a carboxypetidase M inhibitor (CPMi) or activated in media without any addition as a control. The effect of antagonist treatment was assessed in non-activated T cells, as well as T cells activated with anti-CD3, anti-CD3/28, or anti-CD3/46 (black bars). FIG. 12A presents bar graphs showing that CPMi increased secretion of IL-17 by resting and activated CD4+ T cells in control cells, untreated cells, and in the presence of the C5aR antagonist, similar to the effect that was caused by the dual antagonist in the absence of CPMi (left panels). An effect on IFN-γ may also be present. Inhibition of T cell-expressed CPM can thus impair Th1 ‘shut down’. FIG. 12B presents bar graphs showing that CPMi caused increased secretion of IL-6 by resting and activated CD4+ T cells in control cells, untreated cells, and in the presence of the C5aR antagonist, similar to the effect that was caused by the dual antagonist in the absence of CPMi (left panels). The interpretation of these data is that the CPM inhibitor inhibited autocrine production of C5adesArg, thereby reducing the negative regulatory effects that would otherwise result from such production. The resulting effect on release of IL-17 and IL-6 is similar to that achieved by C5L2 blockade.

### Example 7: C5adesArg Partially Rescues Carboxypeptidase M Inhibitor-Mediated Increase in IFN-γ Production by CD4+ T Cells

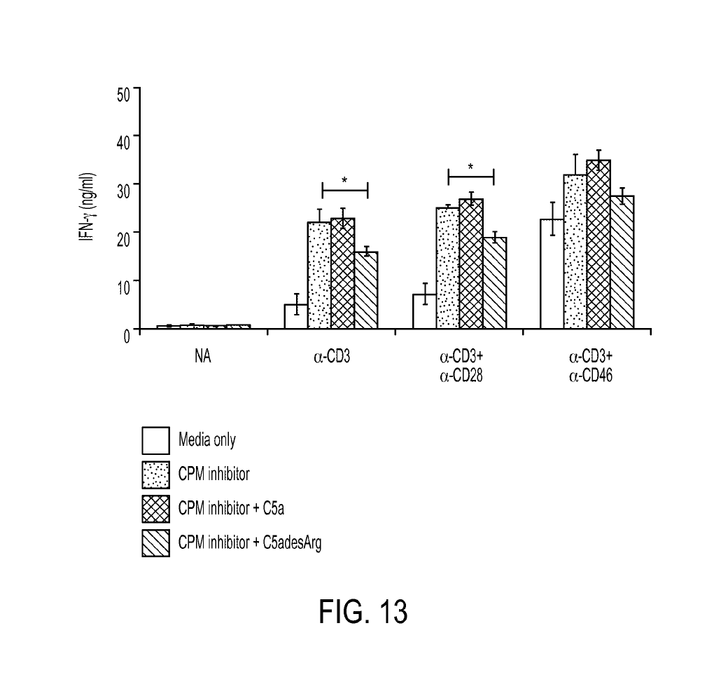

The ability of serum-purified C5adesArg to rescue carboxypeptidase M inhibitor (CPM)-mediated increase in IFN-γ production by CD4+ T cells was determined. Purified human CD4+ T cells were activated in media, or in media with the addition of a CPM inhibitor with or without either serum-purified C5a or C5adesArg. IFN-γ production by cells was assessed 24 h post activation using the CBA Cytokine Bead Array. As shown in FIG. 13, CPMi increased IFN-γ production under all activation conditions. However, the increase in IFN-γ production was markedly lower when C5adesArg was present than when C5a was present and also markedly lower than in the absence of both C5a and C5adesArg. Addition of purified C5adesArg but not C5a reduced CPM inhibitor-induced increase in IFN-γ by about 25%. These results further confirm that C5L2 activation by C5adesArg has distinct biological effects on T cells and that C5L2 activity can be modulated pharmacologically to influence T cell phenotypes and activity.

### Example 8: Blockade of C5L2 on nTregs Inhibits Suppressor Function

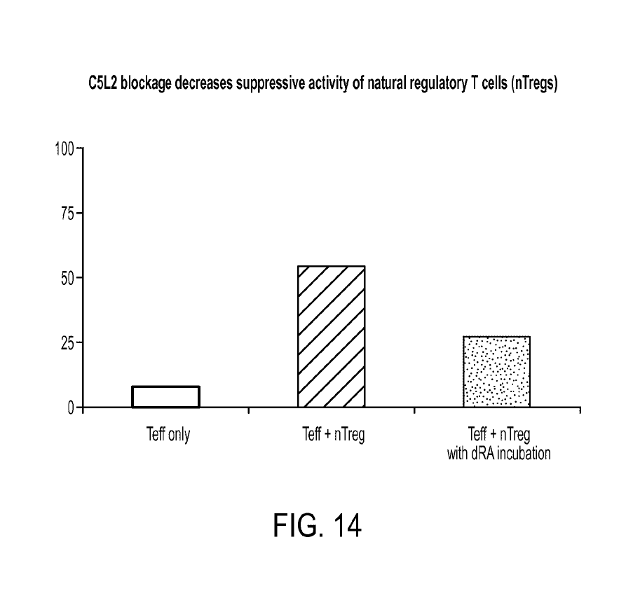

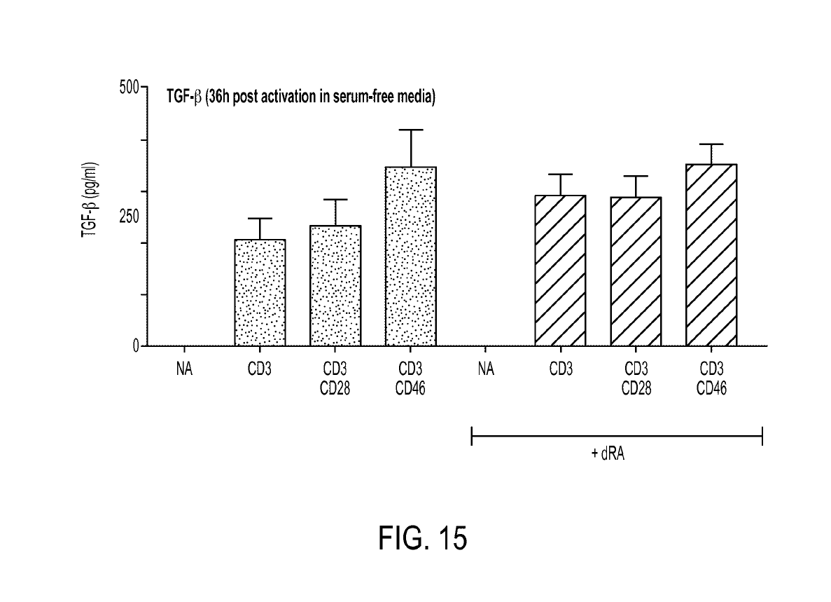

A functional assay was used to assess the effect of C5L2 blockage on suppressive activity of natural regulatory T cells (nTregs). nTregs and effector T cells from a freshly-drawn human blood sample were separated by cell sorting (CD4⁺CD25^{hi}CD127^{lo} Treg cells; CD4⁺CD25^{lo}CD127^{hi} effector T cells). nTreg cells were incubated in media C5ar/C5L2 double antagonist (dRA) for 8 hr and used for a suppression assay via CSFE dilution measurement in 1:1 co-culture and percentage of suppression calculated. As shown in FIG. 14, C5L2 blockage markedly reduced the suppressor function of nTregs.

### Example 9: Further Analysis of C5L2-Mediated Signaling

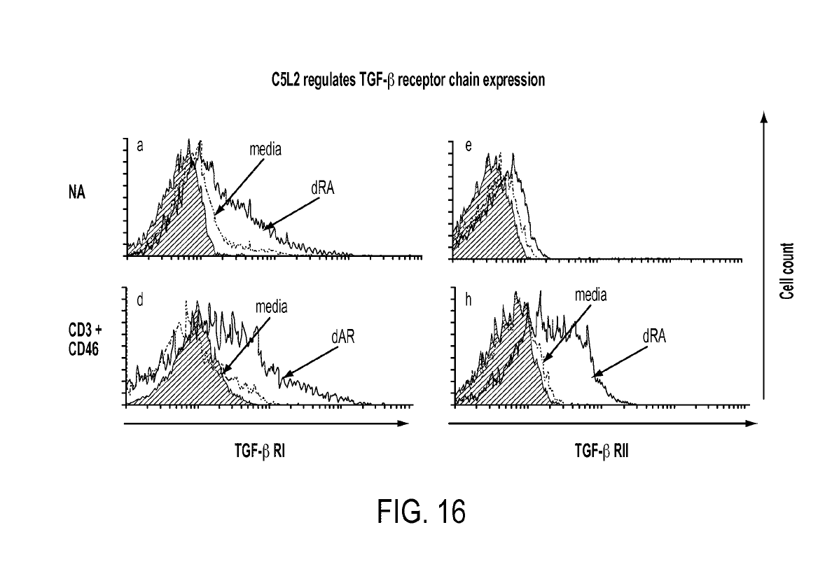

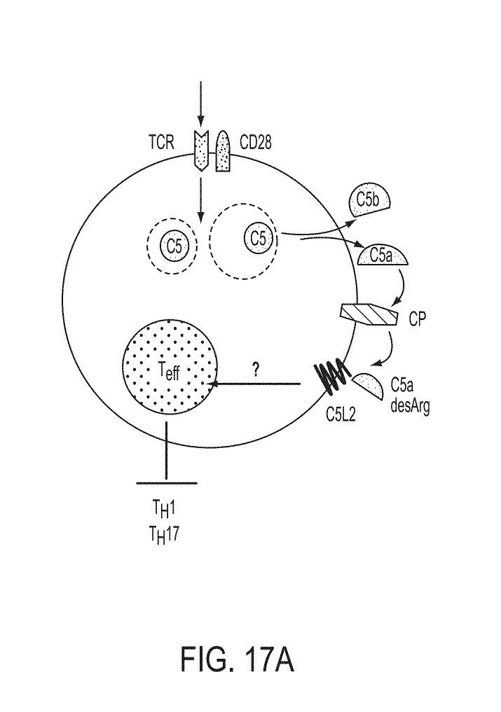

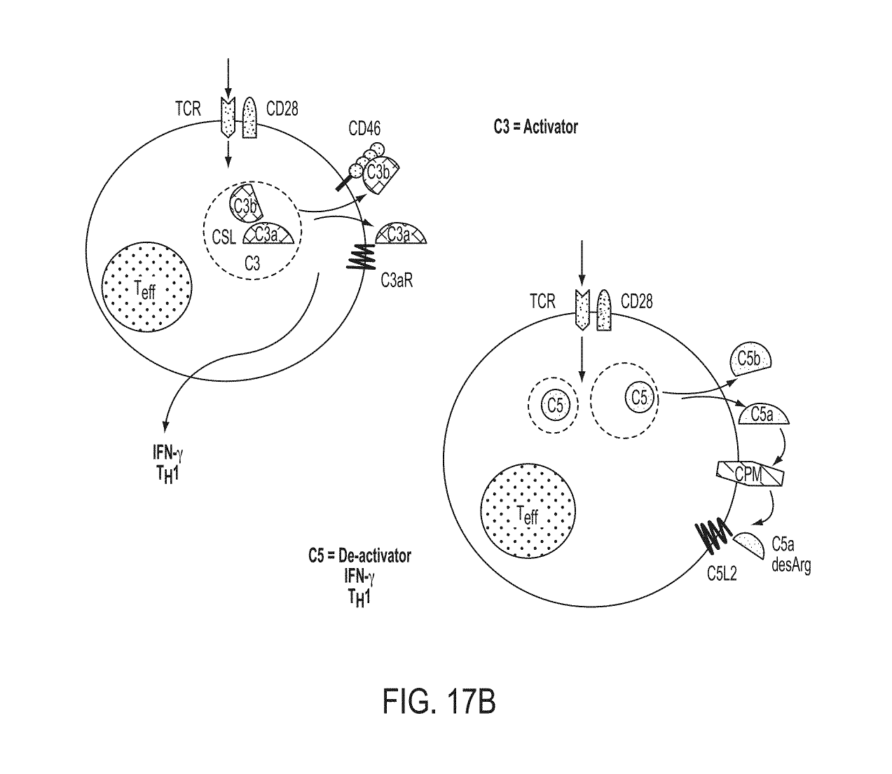

Results described above indicate that C5L2-mediated signals actively contribute to the negative regulation of human Th1 and Th17 responses. To further define C5L2-activated signaling events in CD4+ T cells, gene, miRNA and methylation arrays were performed using T cells from the C5-deficient patient and T cells activated in the presence of the dRA. Initial gene array analyses suggested that the TGF-β signaling pathway may be affected. It is believed that TGF-β is important for IL-17 production but also IFN-γ suppression. TGF-β receptor signaling via autocrine TGF-β production has been implicated as required for control of Th1 responses and prevention of autoimmunity in certain experimental models (Ishigame, et al., Proc Natl Acad Sci USA. (2013) 110(17):6961-6). It was of interest to examine whether C5L2-mediated signals regulate autocrine TGF-β production, TGF-β receptor signaling, or both. Initial results suggested that C5L2 does not markedly regulate autocrine TGF-β production by activated CD4+ T cells (FIG. 15). Additionally, T cells from C5-deficient patients were found produce comparable amounts of TGF-β as those from healthy patients (data not shown). C5L2 was found to regulate TGF-β receptor chain expression. Treatment of CD4+ T cells with the dual antagonist resulted in increased expression of TGF-β receptor chains (FIG. 16). T cells from C5-deficient patients were found to show similar TGF-β receptor dysregulation (data not shown).

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. It will be appreciated that the invention is in no way dependent upon particular results achieved in any specific example or with any specific embodiment. Articles such as “a”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims or from the description above is introduced into another claim. For example, any claim that is dependent on another claim can be modified to include one or more elements, limitations, clauses, or descriptive terms, found in any other claim that is dependent on the same base claim. Furthermore, where the claims recite a composition, it is to be understood that methods of administering the composition according to any of the methods disclosed herein, and methods of using the composition for any of the purposes disclosed herein are included within the scope of the invention, and methods of making the composition according to any of the methods of making disclosed herein are included within the scope of the invention, unless otherwise indicated or unless it would be evident to one of ordinary skill in the art that a contradiction or inconsistency would arise. Methods of treating a subject can include a step of providing a subject in need of such treatment (e.g., a subject who has had, or is at increased risk of having, a disease), a step of diagnosing a subject as having a disease and/or a step of selecting a subject for treatment with an agent. In some embodiments a method of treatment comprises monitoring a subject for a biomarker of a disorder or T cell subset. In some embodiments a method of treatment comprises monitoring a subject for a biomarker and retreating the subject based at least in part on the result of such monitoring.

It is expressly contemplated that each of the various aspects, embodiments, and features thereof described herein may be freely combined with any or all other aspects, embodiments, and features. The resulting aspects and embodiments (e.g., products and methods) are within the scope of the invention. All combinations of the various C5L2 modulators, dosing parameters (e.g., dosing interval, route of administration, etc.), and disorders, e.g., disclosed herein are contemplated in various embodiments. It is to be understood that agents, disorders, methods of administration, and features described at various locations throughout the present application can be in the same embodiment in any combination. Such combinations are expressly encompassed within the scope of the disclosure. It should be understood that headings herein are provided for purposes of convenience and do not imply any limitation on content included below such heading or the use of such content in combination with content included below other headings.

Where elements are presented as lists, it is to be understood that each subgroup of the elements is also disclosed, and any element(s) can be removed from the group. For purposes of conciseness only some of these embodiments have been specifically recited herein, but the invention includes all such embodiments. It should also be understood that, in general, where the invention, or aspects or embodiments of the invention, is/are referred to as comprising particular elements, features, etc., certain embodiments of the invention or aspects of the invention consist, or consist essentially of, such elements, features, etc.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. Any embodiment, aspect, element, feature, etc., of the present invention may be explicitly excluded from the claims. For example, any agent, formulation, formulation component, disorder, subject population or characteristic(s), dosing interval, administration route, or combination thereof can be explicitly excluded.

1.-49. (canceled)

50. A method of inhibiting Th1 and/or Th17 responses by a mammalian T cell, the method comprising contacting the cell with a C5L2 activator.

51. A method of inhibiting production of interleukin-6 (IL-6), interleukin 1 beta (IL-1β), or both by a mammalian T cell or monocyte, the method comprising contacting the cell with a C5L2 activator.

52. The method of claim 50, wherein the cell is a CD4+ T cell.

53.-54. (canceled)

55. A method of increasing suppressive activity of a mammalian nTreg cell, the method comprising contacting the cell with a C5L2 activator.

56. The method of claim 50, wherein the C5L2 activator is an enzyme that processes C5a into C5adesArg or an agent that increases expression or activity of an enzyme that processes C5a into C5adesArg.

57. The method of claim 50, wherein the C5L2 activator is a C5L2 agonist, optionally wherein the C5L2 agonist is selective for C5L2 receptor versus C5a receptor.

58. The method of claim 50, wherein the C5L2 activator comprises an antibody, an engineered non-antibody polypeptide, a peptide, a peptidomimetic, a nucleic acid, or a small molecule.

59. The method of claim 50, wherein the C5L2 activator comprises a variant of C5a, optionally lacking Arg74 of C5a, and further optionally comprising a substitution at position 69 of C5a.

60.-62. (canceled)

63. The method of claim 50, comprising contacting the cell with a C5L2 activator in vivo by administering the C5L2 activator to a mammalian subject.

64.-65. (canceled)

66. The method of claim 50, comprising contacting the cell with a C5L2 activator in vivo by administering the C5L2 activator to a mammalian subject who may benefit from decreased Th1 responses and/or decreased Th17 responses.

67. The method of claim 66, wherein a subject who may benefit from decreased Th1 responses and/or decreased Th17 responses is in need of treatment for an autoimmune disease or an inflammatory disease.

68. The method of claim 51, comprising contacting the cell with a C5L2 activator in vivo by administering the C5L2 activator to a mammalian subject who may benefit from decreased production of IL-6 and/or decreased production of IL-1β, optionally wherein the subject has an IL-6 mediated disease.

69.-79. (canceled)

80. The method of claim 50, wherein the C5L2 activator is physically associated with a clearance reducing moiety, targeting moiety, a cell uptake moiety, a cell-reactive moiety, or a cell membrane binding moiety.

81. (canceled)

82. An agent comprising a C5L2 inhibitor or a C5L2 activator physically associated with a clearance reducing moiety, a targeting moiety, a cell uptake moiety, a cell-reactive moiety, or a cell membrane binding moiety, wherein optionally the C5L2 inhibitor or activator is covalently linked to the clearance reducing moiety, targeting moiety, cell uptake moiety, cell-reactive moiety, or cell membrane binding moiety.

83.-86. (canceled)

87. The agent of claim 82, wherein the cell uptake moiety comprises a cell penetrating peptide.

88.-93. (canceled)

94. A method of treating a subject in need thereof, the method comprising administering the agent or composition of claim 82 to the subject.

95. (canceled)

96. The method of claim 94, wherein the agent or composition comprises a C5L2 activator and the subject is in need of treatment for an autoimmune disease or inflammatory disease.

97. (canceled)

98. The method of claim 50, wherein the C5L2 activator binds to C5L2 and the method comprises screening a plurality of test agents and identifying the C5L2 activator.

99. The method of claim 50, wherein the method comprises screening a plurality of test agents to identify the C5L2 activator, wherein the screening comprises identifying an agent that binds to C5L2 and decreases production of IL-17, IFN-γ, IL-6, IL-1β, or a combination thereof, by a mammalian T cell or monocyte.

100. The method of claim 57, wherein the method comprises screening a plurality of test agents and identifying a C5L2 agonist that is selective for C5L2 versus C5a receptor.
